SEQUENCE LISTING (28 sequences)

SEQ ID NO: 1 - DNA, 25 bp, Artificial Sequence
  Synthetic construct
gugcaagugcaaaccagactttttt

SEQ ID NO: 2 - DNA, 25 bp, Artificial Sequence
  Synthetic construct
gucugguuugcacuugcactttttt

SEQ ID NO: 3 - DNA, 25 bp, Artificial Sequence
  Synthetic construct
agccgaaugucgcagaacctttttt

SEQ ID NO: 4 - DNA, 25 bp, Artificial Sequence
  Synthetic construct
gguucugcgacauucggcutttttt

SEQ ID NO: 5 - DNA, 25 bp, Artificial Sequence
  Synthetic construct
ggauuauaucaaggaggcctttttt

SEQ ID NO: 6 - DNA, 25 bp, Artificial Sequence
  Synthetic construct
ggccuccuugauauaaucctttttt

SEQ ID NO: 7 - DNA, 25 bp, Artificial Sequence
  Synthetic construct
aucgccuaugguuguugactttttt

SEQ ID NO: 8 - DNA, 25 bp, Artificial Sequence
  Synthetic construct
gucaacaaccauaggcgautttttt

SEQ ID NO: 9 - DNA, 25 bp, Artificial Sequence
  Synthetic construct
auacaucccgagaauugcutttttt

SEQ ID NO: 10 - DNA, 25 bp, Artificial Sequence
  Synthetic construct
agcaauucucgggauguautttttt

SEQ ID NO: 11 - DNA, 25 bp, Artificial Sequence
  Synthetic construct
aagccgaaugucgcagaactttttt

SEQ ID NO: 12 - DNA, 25 bp, Artificial Sequence
  Synthetic construct
guucugcgacauucggcuutttttt

SEQ ID NO: 13 - DNA, 21 bp, Artificial Sequence
  Synthetic construct
ggcuacguccaggagcgcacc

SEQ ID NO: 14 - DNA, 21 bp, Artificial Sequence
  Synthetic construct
ugcgcuccuggacguagccuu

SEQ ID NO: 15 - DNA, 25 bp, Artificial Sequence
  Synthetic construct
ggcuacguccaggagcgcatttttt

SEQ ID NO: 16 - DNA, 25 bp, Artificial Sequence
  Synthetic construct
ugcgcuccuggacguagcctttttt

SEQ ID NO: 17 - protein, 9 aa, HIV-1
RKKRRQRRR

SEQ ID NO: 18 - protein, 13 aa, HIV-1
GRKKRRQRRRTPQ

SEQ ID NO: 19 - protein, 11 aa, HIV-1
YGRKKRRQRRR

SEQ ID NO: 20 - protein, 26 aa, Homo sapiens
AAVALLPAVLLALLAPVQRKRQKLMP

SEQ ID NO: 21 - protein, 17 aa, Caiman crocodylus
MGLGLHLLVLAAALQGA

SEQ ID NO: 22 - protein, 23 aa, HIV-1
GALFLGFLGAAGSTMGAPKSKRK

SEQ ID NO: 23 - protein, 16 aa, Drosophila
RQIKIWFQNRRMKWKK

SEQ ID NO: 24 - protein, 5 aa, HIV-1
  VARIANT 1, 5: Xaa = Any Amino Acid
XRGDX

SEQ ID NO: 25 - protein, 24 aa, Influenza
GLFEAIAGFIENGWEGMIDGGGYC

SEQ ID NO: 26 - protein, 27 aa, Artificial Sequence
  Synthetic construct
GWTLNSAGYLLGKINLKALAALAKKIL

SEQ ID NO: 27 - protein, 8 aa, Artificial Sequence
  Synthetic construct
DHQLNPAF

SEQ ID NO: 28 - protein, 8 aa, Homo sapiens
FCYWKTCT

(19) United States
(12) Patent Application Publication
(10) Pub. No.: US 2007/0254850 A1
(43) Pub. Date: Nov. 1, 2007

(54) Methods for Treating and Preventing Apoptosis-Related Diseases Using Rna Interfering Agents

(76) Inventors: Judy Lieberman, Brookline, MA (US); Manjunath Narasimhaswamy, Roslindale, MA (US); Erwei Song, Quincy, MA (US); Sang-Kyung Lee, Brookline, MA (US); Nedim Ince, Iowa City, IA (US); Premlata Shankar, Roslindale, MA (US)

Correspondence Address: DAVID S. RESNICK, 100 SUMMER STREET, NIXON PEABODY LLP, BOSTON, MA, 02110-2131, US

(21) Appl. No.: 10/533,622

(22) PCT Filed: Oct. 30, 2003
(86) PCT No.: PCT/US03/34686

§ 371 Date: Jan. 17, 2006

Related U.S. Application Data
(60) Provisional application No. 60/422,578, filed on Oct. 30, 2002.

(51) Int. Cl. A61K 48/00; C12N 15/867; C12N 5/08

(52) U.S. Cl. 514/44; 424/93.2; 435/456; 435/366

(57) ABSTRACT

The present invention is based, at least in part, on the discovery of compositions and methods useful in the modulation, e.g., inhibition, of gene expression or protein activity. In particular, the present invention is based on novel RNA interfering agents, e.g., siRNA molecules which target apoptosis-related genes or proinflammatory cytokines, and result in reduction, e.g., prolonged reduction, of apoptosis-related gene expression or proinflammatory cytokine expression in cells. Inhibition of apoptosis-related gene expression or protein activity or proinflammatory cytokine expression or protein activity, e.g., by the siRNAs of the invention, inhibits apoptosis-mediated diseases or disorders and proinflammatory cytokine mediated diseases or disorders, including, for example, transplant rejection, hepatitis, liver injury, sepsis, and cancer.

### RELATED APPLICATIONS

This application claims the benefit of U.S. Provisional Application Ser. No. 60/422,578, filed on Oct. 30, 2002, the entire contents of which are incorporated herein by reference.

### GOVERNMENT RIGHTS

This invention was made at least in part with government support under grant nos. AI42510, AI45406, AI49792, and AI45306 awarded by the National Institutes of Health (NIH). The government has certain rights in this invention.

### BACKGROUND OF THE INVENTION

Programmed cell death (PCD), also referred to as apoptosis, is an evolutionary conserved process of eliminating unwanted, damaged, aged and/or misplaced cells during embryonic development and tissue homeostasis in the adult organism (Meier, P., et al. (2000) Nature 407:796; Vaux, D. L. and Korsmeyer, S. J. (1999) Cell 96:245). Apoptosis is an active process with distinctive morphological and biochemical features including membrane blebbing, chromatin condensation and nuclear fragmentation. Several families of molecules have been identified that participate in and/or regulate apoptosis. For example, see the reviews, Konopleva, M., et al. (1999) in Drug Resistance in Leukemia and Lymphoma III, ed. Kaspers, et al. Plenum Pub., New York and Vermeulen, K., et al. (2003) Cell Prolif. 36:165. Caspases are the main effector molecules that are activated and/or regulated by molecules that are responsive to death or survival signals. Caspases are activated or inactivated through a series of intracellular steps, or pathways, in response to these signals, which are themselves subject regulation. There are two major apoptotic pathways defined in mammalian cells, the death receptor pathway and the mitochondrial pathway.

The death receptor pathway is initiated at the cell surface through the FAS receptor (Fas), also referred to as the FAS antigen. Fas is expressed in various tissues (Watanabe-Fukunaga, R., et al., (1992) J. Immunol., 148, 1274-1279) and shares homology with a number of cell-surface receptors including TNF-R and NGF-R. The interaction between Fas and the Fas ligand (FasL) is a key regulator of apoptosis. Since both Fas and FasL are typically membrane-bound, cells expressing either Fas or FasL generally must come into contact with cells expressing the other in order to induce cell death (Rowe, P. M., Lancet, 1996, 347, 1398) which leads to a series of downstream events and the activation of caspases ultimately leading to cell death (Ashkenazi, A. and Dixit, V. M. (1998) Science 281:1305).

Resistance to apoptosis can lead to disorders such as autoimmune disorders, inflammation, or cancer due to the persistence of superfluous, self-specific, infected, or mutated cells (Green, D. R. and Evans, G. (2002) Cancer Cell 1:19; Thompson, C. B. (1995) Science 267:1456; Zornig, M., et al. (2001) Biochim. Biophys. Acta 1551:F1). In contrast, enhanced apoptosis contributes to acute diseases such as infection by toxin-producing microorganisms, ischemia-reperfusion damage or infarction as well as to chronic pathologies such as neurodegenerative disease, neuromuscular disease and AIDS (Mattson, M. P. (2002) Mol. Cell. Biol. 1:120; Rathmell, J. C. and Thompson, C. B. (2002) Cell 109 (Suppl.):S97). Accordingly, modulating programmed cell death can be life-saving.

### SUMMARY OF THE INVENTION

The present invention is based, at least in part, on the discovery of compositions and methods useful in the modulation, e.g., inhibition, of gene expression or protein activity, e.g., apoptosis-related gene expression or protein activity. In particular, the present invention is based on novel RNA interfering agents, e.g. siRNA molecules, which target one or more apoptosis-related genes, e.g., Fas pathway molecules, e.g., Fas or FasL, or proinflammatory cytolines, e.g., IL-1 or TNFα, and result in reduction of apoptosis-related gene expression, e.g., Fas, FasL, or proinflammatory cytokines, e.g., prolonged reduction of apoptosis-related gene expression, e.g. Fas, FasL, or proinflammatory cytokines, in cells, e.g., T cells, hematopoietic cells, hepatocytes, neural cells and/or malignant cells.

In one embodiment, the agent is an RNA which is homologous to a Fas, FasL, IL-1 or TNFα gene, or a fragment thereof. In another embodiment, the agent is an RNA which is homologous to one or more of the apoptosis-related genes, or fragments thereof, listed in Table 1 (below). In another embodiment, the agent is a double-stranded, short interfering RNA (siRNA) which is homologous to any of the apoptosis-related genes listed in Table 1, e.g., a Fas pathway molecule, e.g. Fas or FasL, or a proinflammatory cytokine, e.g., IL-1 or TNFα. In a further embodiment, the agent is a short hairpin RNA (shRNA) which is homologous to any of the apoptosis-related genes listed in Table 1. The siRNA may be about 19 nucleotides to about 28 nucleotides in length, about 19 nucleotides to about 25 nucleotides in length, or e.g. about 21 nucleotides in length. In one embodiment, the siRNA is double stranded and contains a 3′ overhang on each strand. The overhang may be about 1 to about 6 nucleotides on each strand, or e.g., about 2 nucleotides on each strand.

In one embodiment, the RNA interfering agent is a synthetic siRNA. In another embodiment, the first strand of the siRNA comprises the sequence of SEQ ID NO:1 and the second strand of the siRNA comprises the sequence of SEQ ID NO:2. In a further embodiment, the first strand of the siRNA comprises the sequence of SEQ ID NO:3 and the second strand of the siRNA comprises the sequence of SEQ ID NO:4. In a still another embodiment, the first strand of the siRNA comprises the sequence of SEQ ID NO:9 and the second strand of the siRNA comprises the sequence of SEQ ID NO:10. In yet another embodiment, the first strand of the siRNA comprises the sequence of SEQ ID NO:11 and the second strand of the siRNA comprises the sequence of SEQ ID NO:12.

In another embodiment, the RNA interfering agent, e.g., the siRNA, is capable of inducing or regulating degradation of an apoptosis-related mRNA, e.g., a Fas pathway molecule, e.g., Fas or FasL, or a proinflammatory cytokine, e.g., IL-1 or TNFα. In still another embodiment, the RNA interfering agent, e.g., the siRNA inactivates an apoptosis-related gene, e.g., a Fas pathway molecule, e.g., Fas or FasL, or a proinflammatory cytokine, e.g., IL-1 or TNFα, by transcriptional silencing.

In another aspect, the invention provides a vector comprising an RNA interfering agent, e.g., siRNA, which is homologous to an apoptosis-related gene, e.g., a Fas pathway molecule, e.g., Fas or FasL, or a proinflammatory cytokine, e.g., IL-1 or TNFα, and is capable of promoting apoptosis-related gene RNA interference, e.g. Fas, FasL, IL-1 or TNFα RNA interference. In another embodiment, the invention provides a vector comprising a DNA template which encodes an RNA which is homologous to an apoptosis-related gene, e.g., the Fas, FasL, or a proinflammatory cytokine, e.g., IL-1 or TNFα gene, and is capable of promoting apoptosis-related gene RNA interference, e.g., Fas, FasL RNA interference or a proinflammatory cytokine RNA interference, e.g. IL-1 or TNFα RNA interference. In another aspect, the invention provides a cell transfected with a vector comprising a RNA interfering agent, e.g., siRNA, which is homologous to an apoptosis-related gene, e.g., a Fas pathway molecule, e.g. Fas or FasL, or a proinflammatory cytokine, e.g., IL-1 or TNFα, and is capable of promoting apoptosis-related gene RNA interference, e.g., Fas, FasL, or a proinflammatory cytokine RNA interference, e.g., IL-1 or TNFα RNA interference, or a vector comprising a DNA template which encodes an RNA which is homologous to an apoptosis-related gene, e.g., a Fas pathway molecule, e.g., Fas or FasL, or a proinflammatory cytokine, e.g., IL-1 or TNFα, and is capable of apoptosis-related gene RNA interference, e.g., Fas, FasL, IL-1 or TNFα RNA interference.

In a further aspect, the invention provides methods of inhibiting apoptosis in a cell, e.g., a T cell, hematopoietic cell, hepatocyte, neural cell and/or malignant cell, comprising administering to the cell an RNA interfering agent, e.g. siRNA, which modulates apoptosis-related gene expression, e.g., a Fas pathway molecule, e.g. Fas or FasL, or a proinflammatory cytokine, e.g., IL-1 or TNFα gene expression. In still a further aspect, the invention provides methods of inhibiting apoptosis-related gene expression, e.g., Fas pathway molecule, e.g., Fas or FasL, or proinflammatory cytoline, e.g., IL-1 or TNFα gene expression, in a subject comprising administering to the subject an RNA interfering agent, e.g., siRNA, which modulates apoptosis-related gene expression.

In yet another aspect, the invention provides methods of treating a subject having an apoptosis-mediated disease or disorder comprising administering to said subject a therapeutically or prophylactically effective amount of an RNA interfering agent, e.g., siRNA, which modulates apoptosis-related gene expression, e.g., Fas pathway molecule, e.g., Fas or FasL, or proinflammatory cytokine, e.g., IL-1 or TNFα gene expression, so that expression of an apoptosis-related gene, e.g., a Fas pathway molecule, e.g., Fas or FasL, or a proinflammatory cytokine, e.g., IL-1 or TNFα, is inhibited. In one embodiment, the disease or condition is an immune or inflammatory disease, e.g., sepsis, hepatitis, fibrosis, cancer, e.g., cancer of the liver, or transplant rejection. The RNA interfering agent, e.g., siRNA may be administered intravenously, e.g., by a single injection, or may be administered by repeated intravenous injection.

In another aspect, the invention provides methods of preventing allograft, e.g., a hepatic allograft, rejection in an allograft recipient comprising administering to the allograft recipient an RNA interfering agent, e.g., siRNA, which modulates apoptosis-related gene expression, e.g., expression of a Fas pathway molecule. In a related aspect, the invention provides methods of preventing rejection of an allograft, e.g., a hepatic allograft, by an allograft recipient comprising contacting the allograft ex vivo with an RNA interfering agent, e.g., siRNA, which modulates apoptosis-related gene expression, e.g., expression of a Fas molecule.

In yet another aspect, the invention is directed to a method of treating or preventing proinflammatory cytokine mediated disease or disorder in a subject comprising administering to said subject a therapeutically or prophylactically effective amount of an siRNA which modulates proinflammatory cytokine expression so that expression of said proinflammatory cytokine is inhibited. In one embodiment, the proinflammatory cytokine mediated disease or disorder is sepsis. In another embodiment, the proinflammatory cytoline is IL-1 or TNFα, or a fragment thereof.

Other features and advantages of the invention will be apparent from the following detailed description and claims.

### BRIEF DESCRIPTION OF THE DRAWINGS

FIGS. 1A-C Injection of siRNA duplex efficiently silences Fas gene expression in mouse hepatocytes a. Hepatocytes harvested 24 hours after 3 injections of saline or Cy-5-labeled Fas(sequence1)-siRNA were stained with albumin-FITC and analyzed by flow cytometry. A high proportion of hepatocytes take up the duplex siRNA, as indicated. b. RNase protection assay for Fas mRNA expression in hepatocytes from mice that were untreated (lane 1), or injected 24 hours earlier with saline (lane 2), GFP(sequence1)-siRNA (lane 3) or Fas(sequence1)-siRNA (lane 4). Silencing of fas expression in Fas-siRNA treated mice is maintained 5 (lane 5) or 10 days (lane 6) later. Expression of other genes in the fas pathway is unaffected. Similar results were obtained in three independent experiments. Quantitation by densitometry of the Fas/GAPDH levels in 3 mice per condition is graphed. Fas mRNA in hepatocytes is significantly reduced (*P<0.001) at all times in fas-siRNA treated mice compared with control mice. c. Fas immunoblot of lysates from hepatocytes obtained from untreated mice (lane 1), or 24 hours after saline (lane 2), GFP-siRNA (lane 3) or Fas-siRNA (lane 4) injection, and 5 (lane 5) or 10 days (lane 6) after Fas-siRNA injection. Mouse recombinant Fas and FasL proteins serve as positive (P) and negative (N) controls respectively. Similar results were obtained in three independent experiments.

FIGS. 2A-B. In-vivo Fas-siRNA treatment protects mouse hepatocytes from Fas-mediated apoptosis and cytotoxic lysis by conA-activated hepatic mononuclear cells. a. Flow cytometric analysis of FITC-TUNEL staining of primary hepatocytes from untreated, saline injected and Fas(sequence1)-siRNA injected mice exposed in vitro to 500 nM of agonistic anti-Fas mAb. Hepatocytes from untreated mice not exposed to Jo2 mAb serve as negative control. Percentage of FITC-TUNEL+ cells and mean fluorescence intensity (MFI) are indicated. b. Hepatic mononuclear cells from Con A-injected mice lyse hepatocytes from saline treated mice but not Fas(sequence1)-siRNA injected animals. E:T ratio, effector:target ratio. Similar results were obtained in three independent experiments. *P<0.001 at each ratio.

FIG. 3A-E. Fas gene silencing protects mice from fulminant hepatitis and hepatic fibrosis. a Representative liver histology of conA-induced hepatitis in saline, GFP(sequence1)-siRNA and Fas(sequence1)-siRNA injected mice (n=5/group). Livers were stained with hematoxylin and eosin 20 hours after conA injection. (Original magnification ×200) b. Serum ALT and AST in saline, GFP-siRNA and Fas-siRNA injected mice (n=5/study group) measured 20 hours after conA injection. c. Representative liver histology one week after 6 weekly injections of ConA in mock-treated, GFP(sequence1)-siRNA and Fas(sequence1)-siRNA injected mice (n=3/group). (Original magnification ×100). Livers of Fas-siRNA-treated mice were spared the development of bridging fibrosis. d. Hepatic hydroxyproline and serum procollagen type III (PIIINP), indicators of ongoing fibrosis, were normal in Fas-siRNA-injected mice (n=3/group), but elevated in mock-treated and GFP-siRNA-treated mice one week after the last conA injection in the chronic hepatitis model. (#, P<0.05; Φ, P<0.01, compared with control groups). e. Survival advantage of Fas-siRNA injected mice compared to saline or GFP-siRNA treated mice after challenge by intraperitoneal injection with Fas antibody and observation for 10 days before sacrifice. RPA analysis at left (representative data from two independent experiments) shows specific silencing of fas expression in hepatocytes of mice treated with fas-siRNA sequences 1, 5, and 6 and partial silencing with fas sequence 2. The ratio of the fas/GAPDH signal has been normalized to that of mock-treated mice. Sequences that silenced by at least 80% protected against fulminant hepatitis, but fas sequences that did not silence or only inefficiently silenced provided no protection. *P<0.0001.

### DETAILED DESCRIPTION OF THE INVENTION

The present invention is based, at least in part, on the discovery of compositions and methods useful in the modulation, e.g., inhibition, of gene expression or protein activity, e.g., apoptosis-related gene expression or protein activity or proinflammatory cytokine expression or activity. In particular, the present invention is based on novel RNA interfering agents, e.g., small interfering RNA (siRNA) molecules which target apoptosis-related genes, e.g., Fas pathway molecules, e.g., Fas or FasL, or proinflammatory cytokines, e.g., IL-1 or TNFα, and result in reduction, e.g., prolonged reduction, of apoptosis-related gene expression, e.g., Fas pathway molecule, e.g., Fas or FasL, or proinflammatory cytokine, e.g., IL-1 or TNFα gene expression, in cells, e.g., T cells, hematopoietic cells, hepatocytes, neural cells and/or malignant cells. In yet another embodiment, the RNA interfering agents of the invention may be administered to a subject to treat, e.g., therapeutically or prophylactically, an apoptosis-related disease or disorder, e.g., an immune or inflammatory disease, e.g., hepatitis, fibrosis, cancer, e.g., cancer of the liver, cirrhosis, transplant rejection, or sepsis.

As used herein, the term “apoptosis” also referred to as programmed cell death (PCD), is the death of a cell characterized by features including, but not limited to, condensation of nuclear heterochromatin, cell shrinkage, cytoplasmic condensation, and in a later stage of apoptosis, endonuclease mediated cleavage of the DNA of the cell into discrete fragments. Upon electrophoretic analysis of the DNA of a cell in which apoptosis has occurred, a characteristic “ladder” of discrete DNA fragments may be apparent.

As used herein, an “apoptosis-related gene” or “apoptosis-related molecule”, includes any upstream or downstream molecule that is involved in transducing or modulating an apoptotic signal, e.g., molecules involved in or related to apoptotic or anti-apoptotic pathways known to the skilled artisan (see, e.g., Konopleva, M. et al. Drug Resistance in Leukemia and Lymphoma III, Chapter 24 (Kaspers et al. eds. 1999, incorporated herein by reference).

Apoptosis-related genes include, but are not limited to, those molecules listed in Table 1 (below). See also, Siegel, R. M., and Fleisher, T. A. (1999) J. Allergy Clin. Immunol. 103:729-738, incorporated herein by reference. The molecules listed in Table 1 are identified by gene name as well as GenBank GI number. The sequence information included in each GenBank record is incorporated herein by reference.

As used herein, an apoptosis-related gene may be a “pro-apoptotic gene,” which promotes apoptosis or cell death, including, but not limited to, Bcl-Xs, Bax, Fas, FasL, and caspases. An apoptosis-related gene may also be an “anti-apoptotic gene” which inhibits apoptosis or cell death, including, but not limited to, Bcl-2, Bcl-x1, and IAP molecules.

Apoptosis-related genes include, but are not limited to, Fas pathway molecules, e.g., Fas, FasL, and TNF-R1; caspases, e.g., Group I caspases, Group II caspases, and Group III caspases; mitochondrial pathway molecules, e.g., Bcl family molecules, e.g., Bcl-2, Bcl-x1, BclXs, bag, bcl-w, A1, Mcl-1, Bax/Bak-like proteins, Bax, Bak, Bok (Mtd), BCL-Xs, Bcl rambo, bcl-G1, Bik, Blk, Nbk, Bad, Hrk/DP5, Bid, Bim (Bod), Noxa, Puma/Bbc3, Bmf, BNip1, BNip2, BNip3, Nix, Hrk (DP5), Noxa, Puma, p193, Bmf, Bcl-G BCL-interacting proteins; FOXO family members, e.g., FOXO1a and FOXO3a; JAK/STAT family members, e.g., JAK1, JAK2, Stat1, Stat2, Stat 3, Stat4, Stat5a, Stat5b, and Stat6, etc.; and Kinase family members, e.g., cyclin-dependent kinases, mitogen activated protein kinases (MAP kinases).

Other apoptosis-related molecules include any molecule which is upstream or downstream of any apoptotic or anti-apoptotic pathway or is activated or inactivated by any apoptosis-related molecule (see Table 1, for example).

As used herein an “apoptosis-related gene activity” includes, but is not limited to, e.g., Fas activity, e.g., apoptosis, modulation of inflammation, or modulation of an immune response.

Fas Pathway Molecules

Fas pathway molecules include any molecule involved in or related to a pathway leading to apoptosis or PCD induced by Fas. Fas pathway molecules include, but are not limited to Fas, the Fas ligand (FasL), and members of the TNFR superfamily of receptors. FADD, caspase 8, bid, and caspase 3 are also included as Fas pathway molecules. Fas pathway molecules may also be included in other groups as defined herein.

The Fas pathway induces apoptosis by ligation of the Fas receptor on cells by FasL. The Fas receptor, also known as APO-1 or CD95, is a member of the TNFR superfamily of receptors. Other members of the TNFR family include TNF-R1, DR-3, DR-4 and DR-5, each with death domains that directly initiate apoptosis (see Table 1). Binding of FasL to the Fas receptor then leads to aggregation of the receptor on the cell membrane and specific recruitment of intracellular signaling molecules known as DISC, or death-inducing signal complex. The adaptor protein, FADD, binds to the intracellular death domain of Fas which leads to the recruitment of caspase-8, also known as FLICE or MACH. Fas-induced cell death may activate a pathway that alters mitochondrial permeability transition.

Fas receptor engagement is accompanied by an infiltration of inflammatory cells and secondary necrosis and also provokes inflammation, e.g., hepatic inflammation, by inducing expression of cellular chemokines, e.g., hepatic chemokines, that recruit and activate immune cells leading to cell, e.g., hepatocyte, death in a proinflammatory milieu. Accordingly, inhibition of apoptosis-related gene expression or protein activity, e.g., Fas expression or protein activity, e.g., by the siRNAs of the invention, inhibits apoptosis, inflammation, and immune response.

Caspases

Caspase molecules include any enzyme which functions as a cysteine protease and acts as an effector of apoptosis. Caspase molecules are described in Nicholson, D. W. (1999) Cell Death Differentiation 6(11):1028, incorporated herein by reference). Caspases are present as inactive pro-enzymes, most of which are activated by proteolytic cleavage. Caspase molecules include, but are not limited to Group I caspases (caspase 1, 4, and 5); Group II caspases (caspase-2, -3, and -7); Group III caspases (caspase -6, -8, -9, and -10), FLIP (FLICE-inhibitory protein); and Caspase-9, -10, -11, -12, and -13. Caspase-8, caspase-9, and caspase-3 are situated at pivotal junctions in apoptotic pathways.

Mitochondrial Pathway Molecules

The mitochondrial pathway is initiated within cells through the release of cytochrome c and other proteins into the cytoplasm which then activate caspases, which in turn cleave a set of cellular proteins and promote the death program (Thornberry, N. A. and Lazebnik, Y. (1998) Science 281:1312; Wang, X. (2001) Genes Dev. 15:2922). The Bcl-2 family of proteins regulate these mitochondrial changes during apoptosis and necrosis (Adams, J. M. and Corey S. (1992) Science 281:1322; Tsujimoto, Y. and Shimizu, S. (2000a) FEBS Lett. 466:6).

The bcl-2 family of proteins consists of both inhibitors of apoptosis (anti-apoptotic molecules) and promoters of apoptosis (pro-apoptotic molecules). Bcl-2 family proteins share regions of conserved sequence similarity termed as BH domains (bcl-2 homology domains) with all members sharing at least one of the four identified BH domains (BH1-4). Bcl-2 itself is an anti-apoptotic protein which promotes cell survival. Other anti-apoptotic bc1 family members include, for example, bcl-xL, bag, bcl-w, A1, and Mcl-1. There are two pro-apoptotic subgroups within the bcl family. One pro-apoptotic subgroup has multi-domain homology and includes, for example, Bax/Bak-like proteins, Bax, Bak, Bok (Mtd), BCL-Xs, Bcl rambo, and bcl-G1. The second pro-apoptotic subgroup has BH3-only homology and includes, for example, Bik, Blk, Nbk, Bad, Hrk/DP5, Bid, Bim (Bod), Noxa, Puma/Bbc3, Bmf, BNip1, BNip2, BNip3, Nix, Hrk (DP5), Noxa, Puma, p193, Bmf, and Bcl-G. Mitochondrial pathway molecules also include BCL-interacting proteins.

FOXO Family Members

The FOXO family of forkhead transcription factors includes of Foxo3a (FKHRL1), Foxo1 (FKHR), and Foxo4 (AFX), all of which are downstream effectors of the PTEN/PI3K/AKT pathway, being directly phosphorylated and thereby inactivated (via retention in the cytoplasm) by the protein kinase AKT (Tran, H., et al. 2003. Sci STKE 2003, RE5; Ogg, S. and Ruvkun, G. 1998. Mol Cell 2:887; Paradis, S. and Ruvkun, G. 1998. Genes Dev 12,:2488; Dorman, J. B., et al. 1995. Genetics 141:1399, incorporated herein by reference).

JAK/STAT Family Members

STAT (Signal Transducer and Activator of Transcription) molecules include molecules which are capable of transducing a signal from a cytokine receptor to a transcription regulatory element of DNA. STATs play a fundamental role in normal cell signaling in response to cytokines and growth factors. After binding of a cytokine or growth factor to the receptor on cell surface, there is either activation of cytoplasmic tyrosine kinases (particularly JAK or Src kinase families) or activation of receptor-intrinsic tyrosine kinase activity (such as those for EGF or PDGF). Via whichever route, tyrosine phosphorylation leads to activation of STAT monomers initially, which then form dimers through interaction of particular domains called SH-2. The dimers then translocate to the nucleus, and bind to STAT-specific DNA consensus motifs called gamma-activated sites (GAS), of target genes to induce transcription. There are currently seven known members of the STAT family. Examples of JAK/STAT family members are set forth in Table 1.

Kinase Family Members

Kinase family members include any enzyme capable of phosphorylation of a substrate. Kinase family members contain a catalytic domain, and have been classified based on this domain (see, for example, S. Hanks and A. M. Quinn. (1991) Methods in Enzymology 200, 38-62, incorporated herein by reference). Kinases having closely-related catalytic domains tend also to: 1) be similar in overall structural topology, 2) have similar modes of regulation, and 3) have similar substrate specificities. Examples of kinase family members are set forth in Table 1.

An “apoptosis-mediated disease or disorder” or an “apoptosis-related disease or disorder” includes any disease, disorder, or infection in which onset or development of, e.g., progression, is related to or modulated by either inappropriate or excessive apoptosis or cell death or by decreased apoptosis or cell death, e.g., T cell, hematopoietic cell, hepatocyte, neural cell and/or tumor cell death.

In one embodiment, an apoptosis-mediated disease or disorder is related to or mediated by one or more pro-apoptotic genes in which inhibition or expression of the pro-apoptotic gene leading to prevention or inhibition of apoptosis would be beneficial, e.g., infection (e.g., viral, bacterial, or parasitic), autoimmune diseases and disorders, and graft versus host disease.

Examples of apoptosis-mediated diseases or disorders include, but are not limited to, infectious diseases or disorders, immune diseases or disorders, inflammatory diseases or disorders, diseases or disorders which cause liver injury or damage, including hepatocyte injury or damage, e.g., acute and chronic liver injury induced by viral and autoimmune hepatitis, fibrosis, a variety of liver diseases, such as immune related liver diseases, including acute and chronic liver failure, hepatitis, e.g., HBV, HCV, fulminant hepatitis, alcohol induced hepatitis, cholestatic hepatitis, Wilson's disease, and autoimmune hepatitis, and transplant rejection, e.g., liver transplant rejection.

Examples of inflammatory or immune system diseases or disorders, include, but are not limited to sepsis, disseminated intravascular coagulation, viral infection, inflammatory bowel disease, ulcerative colitis, leukocyte adhesion deficiency II syndrome, peritonitis, chronic obstructive pulmonary disease, lung inflammation, asthma, acute appendicitis, nephritis, amyloidosis, chronic bronchitis, sarcoidosis, scleroderma, lupus, polymyositis, Reiter's syndrome, psoriasis, pelvic inflammatory disease, inflammatory breast disease, orbital inflammatory disease, immune deficiency disorders (e.g., HIV, common variable immunodeficiency, congenital X-linked infantile hypogammaglobulinemia, transient hypogammaglobulinemia, selective IgA deficiency, chronic mucocutaneous candidiasis, severe combined immunodeficiency), and autoimmune diseases or disorders.

Examples of autoimmune diseases or disorders include multiple sclerosis, insulin dependent diabetes mellitus, arthritis (e.g., rheumatoid arthritis, juvenile rheumatoid arthritis, osteoarthritis), myesthenia gravis, myocarditis, Guillan-Barre Syndrome, systemic lupus erythematosis, autoimmune thyroiditis, dermatitis, psoriasis, Sjögren's Syndrome, alopecia areata, Crohn's disease, aphthous ulcer, iritis, conjunctivitis, keratoconjunctivitis, ulcerative colitis, allergy, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, cirrhosis, e.g., primary biliary cirrhosis, uveitis posterior, and interstitial lung fibrosis.

As used herein, the term “infectious disease or disorder” is defined as any disease, disorder, or infection which is caused by or related to infection by any infectious agent. For example, infectious diseases or disorders include diseases or disorders caused by or related to infection by a viral infectious agent, bacterial infectious agent, fungal infectious agent, or protozoal infectious agent. Examples of infectious diseases or disorders include, but are not limited to diseases or disorders caused by or related to a viral infectious agent, e.g., HIV, AIDS-related dementia, AIDS-related cancers such as Kaposi's sarcoma, non-Hodgkin's lymphoma, primary central nervous system lymphoma, and invasive squamous cell cancer, AIDS-related diseases or disorders, viral infections including, but not limited to CMV, RSV, HSV, yellow fever virus, dengue fever virus, Japanese encephalitis virus, Murray Valley encephalitis, polioviruis, influenza, rhinovirus, west nile virus, Ebola virus, foot and mouth virus, cytomegalovirus (esp. Human), Rotavirus, Epstein-Barr virus, Varicella Zoster Virus, paramyxoviruses: Respiratory Syncytial virus, parainfluenza virus, measles virus, mumps virus, or influenza virus, human papilloma viruses (for example HPV6, 11, 16, 18 and the like), other sexually transmitted diseases such as, but not limited to hepatitis, e.g., HBV, HCV, HGV, and herpes (HSV-2).

Other examples of apoptosis-mediated diseases and disorders are pulmonary fibrosis, toxic epidermal necrolysis, multiple sclerosis, ulcerative colitis, Sjogren's syndrome, Hashimoto's thyroiditis, and Helicobacter pylori-associated chronic gastritis.

In another embodiment, an apoptosis-mediated disease or disorder is mediated by one or more anti-apoptotic genes in which inhibition of expression of the anti-apoptotic gene resulting in increased or enhanced apoptosis would be beneficial, e.g., cancer. Apoptosis-mediated diseases and disorders also include diseases or disorders which are related to anti-apoptotic genes, including, but not limited to, cellular proliferation, growth, differentiation, or migration disorders and diseases or disorders where there is decreased apoptosis or cell death. Such disorders include cancer, e.g., carcinoma, sarcoma, lymphoma or leukemia, examples of which include, but are not limited to, ovarian, lung, breast, endometrial, uterine, hepatic, gastrointestinal, prostate, colorectal, liver, and brain cancer, tumor angiogenesis and metastasis; skeletal dysplasia; and hematopoietic and/or myeloproliferative disorders. The terms “neoplasia,” “hyperplasia,” and “tumor” are often commonly referred to as “cancer,” which is a general name for more than 100 diseases that are characterized by uncontrolled, abnormal growth of cells. As used herein, a “tumor” also includes a normal, benign, or malignant mass of tissue.

In a further embodiment, the present invention provides methods of preventing allograft rejection in an allograft recipient comprising administering to the allograft recipient an RNA interfering agent, e.g., an siRNA, which modulates, e.g., inhibits, apoptosis-related gene, e.g., Fas, expression. The RNA interfering agent, e.g., siRNA, may be administered to the subject prior to the allograft, during the allograft procedure, or after the allograft, or directly to the allograft. Furthermore, the RNA interfering agent, e.g., the siRNA, can be administered to cells, e.g., T cells, hematopoietic cells, hepatocytes, neural cells and/or tumor cells, or tissues, e.g., blood or liver tissue, ex vivo or in vivo. In one embodiment, RNA interfering agents, e.g., the siRNAs of the invention may be delivered regionally via, e.g., hepatic artery or portal vein cannulation.

Cytokines

In addition to apoptosis-related genes, other targets of the RNA interfering agents of the invention include cytolines, e.g., proinflammatory cytokines, e.g., IL-1β and TNFα, and anti-inflammatory cytokines, e.g., CSF2, CSF3, TGFβ.

Proinflammatory cytokine molecules include any immunoregulatory cytokine that accelerates or induces any aspect of inflammation due to, for example, injury, infection or any immunological disease or disorder or in response to apoptosis-related genes. A proinflammatory cytokine may act either as an endogenous pyrogen (e.g., IL1, TNFα), may upregulate the synthesis of secondary mediators and other pro-inflammatory cytokines by both macrophages and mesenchymal cells (including fibroblasts, epithelial and endothelial cells), may stimulate the production of acute phase proteins, or may attract inflammatory cells. Proinflammatory cytokines include, but are not limited to, for example, IL1α, IL1β, and TNFα, LIF, IFNγ, OSM, CNTF, TGFβ, GM-CSF, IL11, IL12, IL17, IL18, IL8, and a variety of other chemokines that chemoattract inflammatory cells. Other examples of cytokines, e.g., proinflammatory cytolines, are included in Table 1.

Anti-inflammatory cytokine molecules include any immunoregulatory cytokine that counteracts any aspect of inflammation, e.g., cell activation or the production of pro-inflammatory cytokines, and thus contributes to the control of the magnitude of the inflammatory responses in vivo. In one embodiment, anti-inflammatory cytokines act by the inhibition of the production of pro-inflammatory cytokines or by counteracting many biological effects of pro-inflammatory mediators in different ways. Anti-inflammatory cytokines include, but are not limited to, for example, IL4, IL10, and IL13. Other anti-inflammatory mediators include IL16, IFNα, TGF, IL1ra, or G-CSF.

Many microorganisms cause death indirectly by activating a poorly controlled immune response. Examples of this are septic shock and disseminated intravascular coagulation during bacterial infection, with gram negative organisms that have lipopolysaccharide (LPS) as a component of their cell walls and with gram positive bacteria through less well characterized cell wall components. Engagement of the LPS receptor on macrophages activates macrophages to synthesize and secrete proinflammatory cytokines, e.g., TNFα and IL-1β. These proinflammatory cytokines at high concentrations induce a vascular leak syndrome, which makes it impossible to maintain the circulating blood volume, thereby leading to shock. TNFα has also been implicated as the responsible factor in hepatic failure during septic shock.

Using RNAi to target proinflammatory cytokine expression before protein synthesis may be effective in treating and/or preventing sepsis. In one embodiment, targeting more than one inflammatory mediator, e.g., more than one proinflammatory cytokine, may be more effective than just targeting one proinflammatory cytokine, e.g., IL-1 or TNFα alone. In another embodiment, delivery vectors are constructed which are capable of targeting more than one cytokine by using an internal ribosome entry site to produce more than one transcript or by constructing a single transcript with more than one complementary sequence separated by a loop.

Moreover, with regard to infection, it may be possible to identify vulnerable individuals at an early enough stage (possibly before exposure or before symptoms of infection are apparent) for preventative intervention. Accordingly, one aspect of the invention provides in vitro and in vivo methods of using the RNA interfering agents of the invention, e.g., siRNA, to silence the expression of proinflammatory cytokines, e.g., IL-1 or TNFα, implicated in various proinflammatory cytokine related diseases and disorders in, e.g., at risk individuals.

Proinflammatory cytokine mediated diseases and disorders include any disease, disorder, or condition which is related to or caused by expression or activity of a proinflammatory cytokine gene or protein. Proinflammatory cytokine related diseases and disorders include, for example, inflammatory diseases and disorders, including sepsis and septic shock, autoimmune diseases and disorders, infectious disease or disorders.

In one embodiment, the RNA interfering agents, e.g., the siRNAs of the invention, have been shown to be taken up actively by cells in vivo following intravenous injection, e.g., hydrodynamic injection, without the use of a vector, illustrating efficient in vivo delivery of the RNA interfering agents, e.g., the siRNAs of the invention. For example, it has been discovered that siRNAs targeted to the Fas gene display efficient, prolonged suppression of Fas mRNA and protein levels in hepatocytes for at least several days, e.g., 5 days, 1 week, 10 days, or 2 or more weeks, after administration. While not intending to be bound by theory, the duration of silencing in hepatocytes suggests that sustained therapeutic silencing in hepatocytes does not require siRNA expression from plasmids or viral vectors. The difference between liver cells and cell lines is likely because hepatocytes are mostly nondividing, so there is no siRNA dilution with cell division. However, the possibility that sustained suppression in hepatocytes might be due to siRNA amplification, which occurs in lower species (Ketting, R. F. et al. (2001) Genes Dev 15, 2654-9; Lipardi, C., (2001) Cell 107, 297-307) (Schwarz, D. S., (2002) Mol Cell 10, 537-48), is not ruled out. Because silencing after duplex siRNA injection is prolonged but not permanent, long-term toxicity, such as lymphoproliferative or autoimmune disease, seen in humans with mutations of fas and in the lpr mouse (Takahashi, T. et al. (1994) Cell 76, 969-76), is of no concern.

Other strategies for delivery of the RNA interfering agents, e.g., the siRNAs or shRNAs of the invention, may also be employed, such as, for example, delivery by a vector, e.g., a plasmid or viral vector, e.g., a lentiviral vector. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

In one embodiment, the RNA interfering agents, e.g., the siRNAs of the invention, can be introduced into cells, e.g., cultured cells, which are subsequently transplanted into the subject by, e.g., transplanting or grafting, or alternatively, can be obtained from a donor (i.e., a source other than the ultimate recipient), and applied to a recipient by, e.g., transplanting or grafting, subsequent to administration of the RNA interfering agents, e.g., the siRNAs of the invention, to the cells. Alternatively, the RNA interfering agents, e.g., the siRNAs of the invention, can be introduced directly into the subject in such a manner that they are directed to and taken up by the target cells, e.g., T cells, hematopoietic cells, hepatocytes, neural cells and/or tumor cells, and regulate or promote RNA interference of the target gene, e.g., apoptosis-related gene, e.g., Fas. The RNA interfering agents, e.g., the siRNAs of the invention, may be delivered singly, or in combination with other RNA interfering agents, e.g., siRNAs, such as, for example, siRNAs directed to viral genes, such as hepatitis viral genes, or siRNAs directed to other cellular genes, e.g., other apoptosis-related genes or cytokines. The RNA interfering agents, e.g., siRNAs of the invention may also be administered in combination with other pharmaceutical agents which are used to treat or prevent apoptosis-mediated disease or disorders, proinflammatory cytokine related diseases or disorders, apoptosis-mediated tissue injury, e.g., liver or hepatocyte injury caused by or related to apoptosis-related gene activity, apoptosis-mediated inflammation, or an apoptosis-mediated immune response, e.g., anti-viral agents such as interferon treatment, anti-inflammatory agents, cancer therapeutics, or immunosuppressive agents.

As used herein, a “hematopoietic cell” is a blood cell, e.g., erythrocytes, leukocytes including granulocytes, lymphocytes, macrophages and monocytes, platelets, and/or dendritic cells. A “T cell”, as used herein, is intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a CD4+ T cell, CD8+ T cell, CD4+CD8+ T cell, CD4−CD8− T cell, or any other subset of T cells.

Tumor cells can be obtained, for example, from a solid tumor of an organ, such as a tumor of the lung, liver, breast, colon, bone etc. Malignancies of solid organs include, but are not limited to, carcinomas, sarcomas, melanomas and neuroblastomas, The tumor cells can also be obtained from a blood-borne (i.e. dispersed) malignancy such as a lymphoma, a myeloma or a leukemia.

Neural cells are intended to include nerve cells as well as supporting cells, e.g., neuroglial cells, e.g., Schwann cells.

An “RNA interfering agent” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), and small molecules which interfere with or inhibit expression of a target gene by RNA interference (RNAi).

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which fimctions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April;9(4):493-501, incorporated be reference herein).

In one embodiment, the siRNA may target a specific genetic mutation in a target gene. In another embodiment, the siRNA may target a sequence which is conserved between one or more target genes.

The target gene or genomic sequence may be a viral gene or genomic sequence or a cellular gene or genomic sequence. An siRNA may be substantially homologous to the target gene or genomic sequence, or a fragment thereof. As used herein, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues may be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives.

The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated.

Various aspects of the invention are described in further detail in the following subsections:

I. Short Interfering RNAs (siRNAs) of the Invention

In particular, the present invention relates to siRNA molecules of about 15 to about 40 or about 15 to about 28 nucleotides in length, which are homologous to an apoptosis-related gene, e.g., a Fas pathway molecule, e.g., Fas or FasL, or a proinflammatory cytokine, e.g., IL-1 or TNFα, and mediate RNAi of an apoptosis-related gene, e.g., a Fas pathway molecule, e.g., Fas or FasL, or a proinflammatory cytokine, e.g., IL-1 or TNFα. Preferably, the siRNA molecules have a length of about 19 to about 25 nucleotides. More preferably, the siRNA molecules have a length of about 19, 20, 21, or 22 nucleotides. The siRNA molecules of the present invention can also comprise a 3′ hydroxyl group. The siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of the RNA molecule has a 3′ overhang from about 0 to about 6 nucleotides (e.g., pyrmidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the RNA molecule is double stranded, one strand has a 3′ overhang and the other strand can be blunt-ended or have an overhang. In the embodiment in which the RNA molecule is double stranded and both strands comprise an overhang, the length of the overhangs may be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

A. Design and Preparation of siRNA Molecules

Synthetic siRNA molecules, including shRNA molecules, of the present invention can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule of the present invention can be selected from a given target gene sequence, e.g., an apoptosis-related gene, e.g., a Fas pathway molecule, e.g., Fas or FasL, or a proinflammatory cytokine, e.g., IL-1 or TNFα, beginning from about 25 to 50 nucleotides, from about 50 to 75 nucleotides, or from about 75 to 100 nucleotides downstream of the start codon. Nucleotide sequences may contain 5′ or 3′ UTRs and regions nearby the start codon. One method of designing a siRNA molecule of the present invention involves identifying the 23 nucleotide sequence motif AA(N19)TT (where N can be any nucleotide) and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search may be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23 nucleotide sequence motif. The use of symmetric 3′ TT overhangs may be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis companies such as Oligoengine®, may also be used to select siRNA sequences against EST libraries to ensure that only one gene is targeted.

II. Delivery of RNA Interfering Agents

Methods of delivering RNA interfering agents, e.g., an siRNA of the present invention, or vectors containing an RNA interfering agent, e.g., an siRNA of the present invention, to the target cells, e.g., T cells, hematopoietic cells, hepatocytes, neural cells and/or tumor cells, for uptake include injection of a composition containing the RNA interfering agent, e.g., an siRNA, or directly contacting the cell, e.g., a T cell, a hematopoietic cell, a hepatocyte, a neural cell and/or a tumor cell, or an organism, with a composition comprising an RNA interfering agent, e.g., an siRNA. In another embodiment, RNA interfering agents, e.g., an siRNA may be injected directly into the portal vein or hepatic artery. Administration may be by a single injection or by two or more injections.

A viral-mediated delivery mechanism may also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Other methods of introducing siRNA molecules of the present invention to target cells, e.g., T cells, hematopoietic cells, hepatocytes, neural cells and/or tumor cells, include a variety of art-recognized techniques including, but not limited to, calcium phosphate or calcium chloride co-precipitation, DEAE-dextran-mediated transfection, lipofection, or electroporation as well as a number of commercially available transfection kits (e.g., OLIGOFECTAMINE® Reagent from Invitrogen) (see, e.g. Sui, G. et al. (2002) Proc. Natl. Acad. Sci. USA 99:5515-5520; Calegari, F. et al. (2002) Proc. Natl. Acad. Sci., USA Oct. 21, 2002 [electronic publication ahead of print]; J-M Jacque, K. Triques and M. Stevenson (2002) Nature 418:435-437; and Elbashir, S. M. et al. (2001) supra). Suitable methods for transfecting a target cell, e.g., a T cell, a hematopoietic cell, a hepatocyte, a neural cell and/or a tumor cell, can also be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals. The efficiency of transfection may depend on a number of factors, including the cell type, the passage number, the confluency of the cells as well as the time and the manner of formation of siRNA- or shRNA-liposome complexes (e.g., inversion versus vortexing). These factors can be assessed and adjusted without undue experimentation by one with ordinary skill in the art.

The RNA interfering agents, e.g., the siRNAs or shRNAs of the invention, may be introduced along with components that perform one or more of the following activities: enhance uptake of the RNA interfering agents, e.g., siRNA, by the cell, e.g. T cell, hematopoietic cell, hepatocyte, neural cell and/or tumor cell, inhibit annealing of single strands, stabilize single strands, or otherwise facilitate delivery to the target cell and increase inhibition of the target gene, e.g., an apoptosis-related gene, e.g., a Fas pathway molecule, e.g., Fas or FasL, or a proinflammatory cytokine, e.g. IL-1 or TNFα.

The RNA interfering agents, e.g., siRNA, may be directly introduced into the cell, e.g., a T cell, a hematopoietic cell, a hepatocyte, a neural cell and/or a tumor cell, or introduced extracellularly into a cavity, interstitial space, into the circulation of an organism, introduced orally, or may be introduced by bathing a cell or organism in a solution containing the RNA interfering agent, e.g., an siRNA. RNA interfering agents, e.g., an siRNA, may also be introduced into cells via topical application to a mucosal membrane or dermally. Vascular or extravascular circulation, the blood or lymph system, and the cerebrospinal fluid are also sites where the agents may be introduced.

A further method of treating cells with siRNA is an ex vivo method wherein cells, e.g., T cells, hematopoietic cells, hepatocytes, neural cells and/or tumor cells, to be treated with an RNA interfering agent, e.g., an siRNA, are obtained from the individual using known methods and one or more RNA interfering agents that mediate target gene expression are introduced into the cells, which are then re-introduced into the individual. In another embodiment, the cells, e.g., T cells, hematopoietic cells, hepatocytes, neural cells and/or tumor cells, can be obtained from a donor (i.e., a source other than the ultimate recipient), modified by administering the RNA interfering agent(s), and applied to a recipient, again by transplanting or grafting.

For example, cells, e.g., hepatocytes may be obtained from an individual or donor by, generally, removing all or a portion of an organ, e.g., a liver, from which cells, e.g., hepatocytes are removed by in situ perfusion of a collagenase solution. In the case of isolation of hepatocytes from an intact liver, a catheter is inserted into a vein which either leaves or enters the liver, collagenase solution is perfused through and hepatocytes are released. In the case of a liver biopsy, which results in a cut or exposed surface, a small catheter (or catheters) is inserted into vessels on the open or cut surface. Collagenase solution is perfused through the catheterized vessels, resulting in release of hepatocytes. Once removed or isolated, the hepatocytes are plated and maintained under conditions (e.g., on appropriate medium, at correct temperature, etc.) suitable for transfection.

For example, several methods have been described for isolating highly enriched populations of rat hepatocytes and maintaining these cells in culture for extended periods of time. Koch, K. S. and H. L. Leffert, Ann. N.Y. Acad. Sci., 349:111-127 (1980); McGowan, J. A. et al., J. Cell. Physiol., 108:353-363 (1981); Bissell, D. M. and P. S. Guzelian, Ann. N.Y. Acad. Sci, 349:85-98 (1981); and Enat, R. et al., Proc. Nat. Acad. Sci., USA, 81:1411-1415 (1984). Such methods can be used to isolate and maintain hepatocytes to be transduced by the methods of the present invention. Hepatocytes can also be prepared using the procedure described in U.S. Pat. No. 5,580,776, with the perfusion mixture described by Leffert (Leffert, H. L. et al., Methods Enzymol., 58:536-544 (1979), the entire contents of which are incorporated herein by reference).

Cells, e.g., T cells, hematopoietic cells, hepatocytes, neural cells and/or tumor cells, containing the incorporated RNA interfering agents of the invention are grown to confluence in tissue culture vessels; removed from the culture vessel; and introduced into the body. This can be done surgically, for example. In this case, the tissue which is made up of transduced hepatocytes capable of expressing the nucleotide sequence of interest is grafted or transplanted into the body. For example, it can be placed in the abdominal cavity in contact with/grafted onto the liver or in close proximity to the liver. Alternatively, the transduced hepatocyte-containing tissue can be attached to microcarrier beads, which are introduced (e.g., by injection) into the peritoneal space of the recipient. This approach has been shown to be successful by transplantation of wild type hepatocytes into a strain of rats (Nagase analbuminemic rats) which are deficient in albumin synthesis and demonstration of moderate levels of albumin in serum of transplanted animals. Direct injection of genetically modified hepatocytes into the liver may also be possible by, e.g., portal vein infusion.

If necessary, biochemical components needed for RNAi to occur can also be introduced into the cells, e.g., T cells, hematopoietic cells, hepatocytes, neural cells and/or tumor cells.

Another aspect of the invention pertains to vectors, for example, recombinant expression vectors, containing a nucleic acid encoding an siRNA of the present invention, e.g., an apoptosis-related siRNA, e.g., a Fas or FasL siRNA, or a proinflammatory cytokine, e.g., related IL-1 or TNFα siRNA. As used herein, the term “vector” refers to a nucleic acid molecule capable of transporting another nucleic acid to which it has been linked. One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g. non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors”, or more simply “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, lentiviruses, adenoviruses and adeno-associated viruses), which serve equivalent functions. In a preferred embodiment, lentiviruses are used to deliver one or more siRNA molecule of the present invention to a cell. Vectors which are useful for the introduction of genetic material to hepatocytes are described in, for example, U.S. Pat. No. 5,580,776.

Within an expression vector, “operably linked” is intended to mean that the nucleotide sequence of interest is linked to the regulatory sequence(s) in a manner which allows for expression of the nucleotide sequence (e.g., in an in vitro transcription/translation system or in a target cell when the vector is introduced into the target cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Furthermore, the RNA interfering agents may be delivered by way of a vector comprising a regulatory sequence to direct synthesis of the siRNAs of the invention at specific intervals, or over a specific time period. It will be appreciated by those skilled in the art that the design of the expression vector can depend on such factors as the choice of the target cell, the level of expression of siRNA desired, and the like.

The expression vectors of the invention can be introduced into target cells to thereby produce siRNA molecules of the present invention. In one embodiment, a DNA template, e.g., a DNA template encoding an apoptosis-related gene, e.g., a Fas pathway molecule, e.g., Fas or FasL, or a proinflammatory cytokine, e.g., IL-1 or TNFα, may be ligated into an expression vector under the control of RNA polymerase III (Pol III), and delivered to a target cell. Pol III directs the synthesis of small, noncoding transcripts which 3′ ends are defined by termination within a stretch of 4-5 thymidines. Accordingly, DNA templates may be used to synthesize, in vivo, both sense and antisense strands of siRNAs which effect RNAi (Sui, et al. (2002) PNAS 99(8):5515).

The expression vectors of the invention may also be used to introduce shRNA into target cells.

As used herein, the term “target cell” is intended to refer to a cell, e.g., a T cell, a hematopoietic cell, a hepatocyte, a neural cell and/or a tumor cell, into which an siRNA molecule of the invention, including a recombinant expression vector encoding an siRNA of the invention, has been introduced. The terms “target cell” and “host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Preferably, a target cell is a mammalian cell, e.g., a human cell. In particularly preferred embodiments, it is a hepatocyte.

It is known that depending upon the expression vector and transfection technique used, only a small fraction of cells may effectively uptake the siRNA molecule. In order to identify and select these cells, antibodies against a cellular target can be used to determine transfection efficiency through immunofluorescence. Preferred cellular targets include those which are present in the host cell type and whose expression is relatively constant, such as Lamin A/C. Alternatively, co-transfection with a plasmid containing a cellular marker, such as a CMV-driven EGFP-expression plasmid, luciferase, metalloprotease, BirA, B-galactosidase and the like may also be used to assess transfection efficiency. Cells which have been transfected with the siRNA molecules can then be identified by routine techniques such as immunofluorescence, phase contrast microscopy and fluorescence microscopy.

Depending on the abundance and the life-time (or turnover) of the targeted protein, a knock-down phenotype, e.g., a phenotype associated with siRNA inhibition of the target gene, e.g., an apoptosis-related gene, e.g., a Fas pathway molecule, e.g., Fas or FasL, or a proinflammatory cytokine, e.g., IL-1 or TNFα, may become apparent after 1 to 3 days, or even later. In cases where no phenotype is observed, depletion of the protein may be observed by immunofluorescence or Western blotting. If the protein is still abundant after 3 days, cells can be split and transferred to a fresh 24-well plate for re-transfection.

If no knock-down of the targeted protein is observed, it may be desirable to analyze whether the target mRNA was effectively destroyed by the transfected siRNA duplex. Two days after transfection, total RNA can be prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable protein may exist in the cell. Multiple transfection in sufficiently long intervals may be necessary until the target protein is finally depleted to a point where a phenotype may become apparent.

RNA interfering agents of the instant invention also include, for example, small molecules which interfere with or inhibit expression of a target gene. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene. Assays to determine expression of the target gene, e.g., an apoptosis-related gene, e.g., a Fas pathway molecule, e.g., Fas or FasL, or a proinflammatory cytokine, e.g., IL-1 or TNFα, and the activity or level of the protein encoded by the target gene, are known in the art. For example, reduced levels of target gene mRNA may be measured by in situ hybridization (Montgomery et al., (1998) Proc. Natl. Acad. Sci., USA 95:15502-15507) or Northern blot analysis (Ngo, et al. (1998)) Proc. Natl. Acad. Sci., USA 95:14687-14692).

Apoptosis-related polypeptide activity, e.g., Fas polypeptide activity, e.g., apoptosis, may also be assayed for by, for example, assays known in the art for cell death or apoptosis, such as, for example, transient transfection assays for cell death genes (as described in, for example, Miura M. et al. (2000) Methods in Enzymol. 322:480-92); assays that detect DNA cleavage in apoptotic cells (as described in, for example, Kauffman S. H. et al. (2000) Methods in Enzymol. 322:3-15); detection of apoptosis by annexin V labeling (as described in, for example, Bossy-Wetzel E. et al. (2000) Methods in Enzymol. 322:15-18); apoptotic nuclease assays (as described in, for example, Hughes F. M. (2000) Methods in Enzymol. 322:47-62); and analysis of apoptotic cells by flow and laser scanning cytometry (as described in, for example, Darzynkiewicz Z. et al. (2000) Methods in Enzymol. 322:18-39).

In another embodiment, the compositions of the invention are provided as a surface component of a lipid aggregate, such as a liposome, or are encapsulated by a liposome. Liposomes, which can be unilamellar or multilamellar, can introduce encapsulated material into a cell by different mechanisms. For example, the liposome can directly introduce its encapsulated material into the cell cytoplasm by fusing with the cell membrane. Alternatively, the liposome can be compartmentalized into an acidic vacuole (i.e., an endosome) and its contents released from the liposome and out of the acidic vacuole into the cellular cytoplasm. In one embodiment the invention features a lipid aggregate formulation of the compounds described herein, including phosphatidylcholine (of varying chain length; e.g., egg yolk phosphatidylcholine), cholesterol, a cationic lipid, and 1,2-distearoyl-sn-glycero3-phosphoethanolamine-polythyleneglycol-2000 (DSPE-PEG2000). The cationic lipid component of this lipid aggregate can be any cationic lipid known in the art such as dioleoyl 1,2,-diacyl trimethylammonium-propane (DOTAP). In another embodiment, polyethylene glycol (PEG) is covalently attached to the compositions of the present invention. The attached PEG can be any molecular weight but is preferably between 2000-50,000 daltons. In one embodiment for targeting macrophages for delivery of an RNA interfering agent, liposomes containing of phosphatidyl serine may be used since macrophage engulfment via the phosphatidyl serine receptor promotes an anti-inflammatory response by increasing TGF-β1 secretion (Huynh, M. L. et al. (2002) J. Cell Biol. 155, 649). Therefore, when the macrophages are successfully transfected, not only will the target genes be silenced, but the macrophage will also be induced to secrete anti-inflammatory cytokines.

In another embodiment, for delivery to a macrophage, a polyG tail, e.g., a 5-10 nucleotide tail, may be added to the 5′ end of the sense strand of the siRNA, which will enhance uptake via the macrophage scavenger receptor.

In another embodiment of the invention, the RNA interfering agents of the invention may be transported or conducted across biological membranes using carrier polymers which comprise, for example, contiguous, basic subunits, at a rate higher than the rate of transport of RNA interfering agents, e.g., siRNA molecules, which are not associated with carrier polymers. Combining a carrier polymer with an RNA interfering agents, e.g., an siRNA, with or without a cationic transfection agent, results in the association of the carrier polymer and the RNA interfering agent, e.g. siRNA. The carrier polymer may efficiently deliver the RNA interfering agent, e.g., siRNA, across biological membranes both in vitro and in vivo. Accordingly, the invention provides methods for delivery of an RNA interfering agent, e.g., an siRNA, across a biological membrane, e.g., a cellular membrane including, for example, a nuclear membrane, using a carrier polymer. The invention also provides compositions comprising an RNA interfering agent, e.g., an siRNA, in association with a carrier polymer.

The term “association” or “interaction” as used herein in reference to the association or interaction of an RNA interfering agent and a carrier polymer, refers to any association or interaction between an RNA interfering agent, e.g., an siRNA, with a carrier polymer, e.g., a peptide carrier, either by a direct linkage or an indirect linkage.

An indirect linkage includes an association between a RNA interfering agent and a carrier polymer wherein said RNA interfering agent and said carrier polymer are attached via a linker moiety, e.g., they are not directly linked. Linker moieties include, but are not limited to, e.g., nucleic acid linker molecules, e.g., biodegradable nucleic acid linker molecules. A nucleic acid linker molecule may be, for example, a dimer, trimer, tetramer, or longer nucleic acid molecule, for example an oligonucleotide of about 2, 3, 4, 5, 6, 7, 8, 9, 10, 11, 12, 13, 14, 15, 16, 17, 18, 19, 20, 21, 22, 23, 24, 25, or more nucleotides in length.

A direct linkage includes any linkage wherein a linker moiety is not required. In one embodiment, a direct linkage includes a chemical or a physical interaction wherein the two moieties, e.g., the RNA interfering agent and the carrier polymer, interact such that they are attracted to each other. Examples of direct interactions include non-covalent interactions, hydrophobic/hydrophilic, ionic (e.g. electrostatic, coulombic attraction, ion-dipole, charge-transfer), Van der Waals, or hydrogen bonding, and chemical bonding, including the formation of a covalent bond. Accordingly, in one embodiment, the RNA interfering agent and the carrier polymer are not linked via a linker, e.g., they are directly linked. In a further embodiment, the RNA interfering agent and the carrier polymer are electrostatically associated with each other.

The term “polymer” as used herein, refers to a linear chain of two or more identical or non-identical subunits joined by covalent bonds. A peptide is an example of a polymer that can be composed of identical or non-identical amino acid subunits that are joined by peptide linkages.

The term “peptide” as used herein, refers to a compound made up of a single chain of D- or L-amino acids or a mixture of D- and L-amino acids joined by peptide bonds. Generally, peptides contain at least two amino acid residues and are less than about 50 amino acids in length.

The term “protein” as used herein, refers to a compound that is composed of linearly arranged amino acids linked by peptide bonds, but in contrast to peptides, has a well-defined conformation. Proteins, as opposed to peptides, generally consist of chains of 50 or more amino acids.

“Polypeptide” as used herein, refers to a polymer of at least two amino acid residues and which contains one or more peptide bonds. “Polypeptide” encompasses peptides and proteins, regardless of whether the polypeptide has a well-defined conformation.

In one embodiment, carrier polymers in accordance with the present invention contain short-length polymers of from about 6 to up to about 25 subunits. The carrier is effective to enhance the transport rate of the RNA interfering agent across the biological membrane relative to the transport rate of the biological agent alone. Although exemplified polymer compositions are peptides, the polymers may contain non-peptide backbones and/or subunits as discussed further below.

In an important aspect of the invention, the carrier polymers of the invention are particularly useful for transporting biologically active agents across cell or organelle membranes, when the RNA interfering agents are of the type that require trans-membrane transport to exert their biological effects. As a general matter, the carrier polymer used in the methods of the invention preferably includes a linear backbone of subunits. The backbone will usually comprise heteroatoms selected from carbon, nitrogen, oxygen, sulfur, and phosphorus, with the majority of backbone chain atoms usually consisting of carbon. Each subunit may contain a sidechain moiety that includes a terminal guanidino or amidino group.

Although the spacing between adjacent sidechain moieties will usually be consistent from subunit to subunit, the polymers used in the invention can also include variable spacing between sidechain moieties along the backbone.

The sidechain moieties extend away from the backbone such that the central guanidino or amidino carbon atom (to which the NH₂ groups are attached) is linked to the backbone by a sidechain linker that preferably contains at least 2 linker chain atoms, more preferably from 2 to 5 chain atoms, such that the central carbon atom is the third to sixth chain atom away from the backbone. The chain atoms are preferably provided as methylene carbon atoms, although one or more other atoms such as oxygen, sulfur, or nitrogen can also be present. Preferably, the sidechain linker between the backbone and the central carbon atom of the guanidino or amidino group is 4 chain atoms long, as exemplified by an arginine side chain.

The carrier polymer sequence of the invention can be flanked by one or more non-guanidino/non-amidino subunits, or a linker such as an aminocaproic acid group, which do not significantly affect the rate of membrane transport of the corresponding polymer-containing conjugate, such as glycine, alanine, and cysteine, for example. Also, any free amino terminal group can be capped with a blocking group, such as an acetyl or benzyl group, to prevent ubiquitination in vivo.

The carrier polymer of the invention can be prepared by straightforward synthetic schemes. Furthermore, the carrier polymers are usually substantially homogeneous in length and composition, so that they provide greater consistency and reproducibility in their effects than heterogenous mixtures.

According to an important aspect of the present invention, association of a single carrier polymer to an RNA interfering agent, e.g., an siRNA, is sufficient to substantially enhance the rate of uptake of an agent across biological membranes, even without requiring the presence of a large hydrophobic moiety in the conjugate. In fact, attaching a large hydrophobic moiety may significantly impede or prevent cross-membrane transport due to adhesion of the hydrophobic moiety to the lipid bilayer. Accordingly, the present invention includes carrier polymers that do not contain large hydrophobic moieties, such as lipid and fatty acid molecules.

In one embodiment, the transport polymer is composed of D- or L-amino acid residues. Use of naturally occurring L-amino acid residues in the transport polymers has the advantage that break-down products should be relatively non-toxic to the cell or organism. Preferred amino acid subunits are arginine (α-amino-delta.-guanidi-novaleric acid) and α-amino-ε-amidinohexanoic acid (isosteric amidino analog). The guanidinium group in arginine has a pKa of about 12.5.

More generally, it is preferred that each polymer subunit contains a highly basic sidechain moiety which (i) has a pKa of greater than 11, more preferably 12.5 or greater, and (ii) contains, in its protonated state, at least two geminal amino groups (NH₂) which share a resonance-stabilized positive charge, which gives the moiety a bidentate character.

Other amino acids, such as α-amino-β-guanidinopropionic acid, α-amino-γ-guanidinobutyric acid, or α-amino-ε-guanidinocaproic acid can also be used (containing 2, 3 or 5 linker atoms, respectively, between the backbone chain and the central guanidinium carbon).

D-amino acids may also be used in the transport polymers. Compositions containing exclusively D-amino acids have the advantage of decreased enzymatic degradation. However, they may also remain largely intact within the target cell. Such stability is generally not problematic if the agent is biologically active when the polymer is still attached. For agents that are inactive in conjugate form, a linker that is cleavable at the site of action (e.g., by enzyme- or solvent-mediated cleavage within a cell) should be included to promote release of the agent in cells or organelles.

Any peptide, e.g., basic peptide, or fragment thereof, which is capable of crossing a biological membrane, either in vivo or in vitro, is included in the invention. These peptides can be synthesized by methods known to one of skill in the art. For example, several peptides have been identified which may be used as carrier peptides in the methods of the invention for transporting RNA interfering agents across biological membranes. These peptides include, for example, the homeodomain of antennapedia, a Drosophila transcription factor (Wang et al., (1995) PNAS USA., 92, 3318-3322); a fragment representing the hydrophobic region of the signal sequence of Kaposi fibroblast growth factor with or without NLS domain (Antopolsky et al. (1999) Bioconj. Chem., 10, 598-606); a signal peptide sequence of caiman crocodylus Ig(5) light chain (Chaloin et al. (1997) Biochem. Biophys. Res. Comm., 243, 601-608); a fusion sequence of HIV envelope glycoprotein gp4114, (Morris et al. (1997) Nucleic Acids Res., 25, 2730-2736); a transportan A-achimeric 27-mer consisting of N-terminal fragment of neuropeptide galanine and membrane interacting wasp venom peptide mastoporan (Lindgren et al., (2000), Bioconjugate Chem., 11, 619-626); a peptide derived from influenza virus hemagglutinin envelop glycoprotein (Bongartz et al., 1994, Nucleic Acids Res., 22, 468 1 4688); RGD peptide; and a peptide derived from the human immunodeficiency virus type-1 (“HIV-1”). Purified HIV-1 TAT protein is taken up from the surrounding medium by human cells growing in culture (A. D. Frankel and C. O. Pabo, (1988) Cell, 55, pp. 1189-93). TAT protein trans-activates certain HIV genes and is essential for viral replication. The full-length HIV-1 TAT protein has 86 amino acid residues. The HIV tat gene has two exons. TAT amino acids 1-72 are encoded by exon 1, and amino acids 73-86 are encoded by exon 2. The full-length TAT protein is characterized by a basic region which contains two lysines and six arginines (amino acids 47-57) and a cysteine-rich region which contains seven cysteine residues (amino acids 22-37). The basic region (i.e., amino acids 47-57) is thought to be important for nuclear localization. Ruben, S. et al., J. Virol. 63: 1-8 (1989); Hauber, J. et al., J. Virol. 63 1181-1187 (1989); Rudolph et al. (2003) 278(13):11411. The cysteine-rich region mediates the formation of metal-linked dimers in vitro (Frankel, A. D. et al., Science 240: 70-73 (1988); Frankel, A. D. et al., Proc. Natl. Acad. Sci USA 85: 6297-6300 (1988)) and is essential for its activity as a transactivator (Garcia, J. A. et al., EMBO J. 7:3143 (1988); Sadaie, M. R. et al., J. Virol. 63: 1 (1989)). As in other regulatory proteins, the N-terminal region may be involved in protection against intracellular proteases (Bachmair, A. et al., Cell 56: 1019-1032 (1989)).

In one embodiment of the invention, the basic peptide comprises amino acids 47-57 of the HIV-1 TAT peptide. In another embodiment, the basic peptide comprises amino acids 48-60 of the HIV-1 TAT peptide. In still another embodiment, the basic peptide comprises amino acids 49-57 of the HIV-1 TAT peptide. In yet another embodiment, the basic peptide comprises amino acids 49-57, 48-60, or 47-57 of the HIV-1 TAT peptide, does not comprise amino acids 22-36 of the HIV-1 TAT peptide, and does not comprise amino acids 73-86 of the HIV-1 TAT peptide. In still another embodiment, the specific peptides set forth in Table 2, below, or fragments thereof, may be used as carrier peptides in the methods and compositions of the invention.

TABLE 2

| | | SEQ |
|---|---|---|
| | | ID |
| Peptide | Sequence | NO: |
| HIV-1 TAT (49-57) | RKKRRQRRR | 17 |
| HIV-1 TAT (48-60) | GRKKRRQRRRTPQ | 18 |
| HIV-1 TAT (47-57) | YGRKKRRQRRR | 19 |
| Kaposi fibroblast | AAV ALL PAV LLA | 20 |
| growth factor | LLA P + VQR KRQ | |
| | KLMP | |
| of caiman | MGL GLH LLV LAA | 21 |
| crocodylus Ig(5) | ALQ GA | |
| light chain | | |
| HIV envelope | GAL FLG FLG AAG | 22 |
| glycoprotein gp41 | STM GA + PKS KRK 5 | |
| | (NLS of the SV40) | |
| Drosophila | RQI KIW FQN RRM | 23 |
| Antennapedia | KWK K amide | |
| RGD peptide | X-RGD-X | 24 |
| influenza virus | GLFEAIAGFIENGWE | 25 |
| hemagglutinin | GMIDGGGYC | |
| envelop | | |
| glycoprotein | | |
| transportan A | GWT LNS AGY LLG | 26 |
| | KIN LKA LAA LAK | |
| | KIL | |
| Pre-S-peptide | (S)DH QLN PAF | 27 |
| Somatostatin (tyr-3- | (S)FC YWK TCT | 28 |
| octreotate) | | |

(s) optional Serine for coupling
italic = optional D isomer for stability

In yet another embodiment, an active thiol at the 5′ end of the sense strand may be coupled to a cysteine reside added to the C terminal end of a basic peptide for delivery into the cytosol (such as a fragment of tat or a fragment of the Drosophila Antennapedia peptide). Internalization via these peptides bypasses the endocytic pathway and therefore removes the danger of rapid degradation in the harsh lysosomal environment, and may reduce the concentration required for biological efficiency compared to free oligonucleotides.

Other arginine rich basic peptides are also included for use in the present invention. For example, a TAT analog comprising D-amino acid- and arginine-substituted TAT(47-60), RNA-binding peptides derived from virus proteins such as HIV-1 Rev, and flock house virus coat proteins, and the DNA binding sequences of leucine zipper proteins, such as cancer-related proteins c-Fos and c-Jun and the yeast transcription factor GCN4, all of which contain several arginine residues (see Futaki, et al. (2001) J. Biol Chem 276(8):5836-5840 and Futaki, S. (2002) Int J. Pharm 245(1-2):1-7, which are incorporated herein by reference). In one embodiment, the arginine rich peptide contains about 4 to about 11 arginine residues. In another embodiment, the arginine residues are contiguous residues.

Subunits other than amino acids may also be selected for use in forming transport polymers. Such subunits may include, but are not limited to hydroxy amino acids, N-methyl-amino acids amino aldehydes, and the like, which result in polymers with reduced peptide bonds. Other subunit types can be used, depending on the nature of the selected backbone.

A variety of backbone types can be used to order and position the sidechain guanidino and/or amidino moieties, such as alkyl backbone moieties joined by thioethers or sulfonyl groups, hydroxy acid esters (equivalent to replacing amide linkages with ester linkages), replacing the alpha carbon with nitrogen to form an aza analog, alkyl backbone moieties joined by carbamate groups, polyethyleneimines (PEIs), and amino aldehydes, which result in polymers composed of secondary amines.

A more detailed backbone list includes N-substituted amide (CONR replaces CONH linkages), esters (CO₂), ketomethylene (COCH₂) reduced or methyleneamino (CH₂NH), thioamide (CSNH), phosphinate (PO₂RCH₂), phosphonamidate and phosphonamidate ester (PO₂RNH), retropeptide (NHCO), transalkene (CR.dbd.CH), fluoroalkene (CF.dbd.CH), dimethylene (CH₂2CH₂), thioether (CH₂S), hydroxyethylene (CH(OH)CH₂), methyleneoxy (CH₂O), tetrazole (CN₂4), retrothioamide (NHCS), retroreduced (NHCH₂), sulfonamido (SO₂NH), methylenesulfonamido (CHRSO₂NH), retrosulfonamide (NHSO₂), and peptoids (N-substituted glycines), and backbones with malonate and/or gem-diaminoalkyl subunits, for example, as reviewed by Fletcher et al. (1998) and detailed by references cited therein. Peptoid backbones (N-substituted glycines) can also be used. Many of the foregoing substitutions result in approximately isosteric polymer backbones relative to backbones formed from α-amino acids.

Polymers are constructed by any method known in the art. Exemplary peptide polymers can be produced synthetically, preferably using a peptide synthesizer (Applied Biosystems Model 433) or can be synthesized recombinantly by methods well known in the art.

N-methyl and hydroxy-amino acids can be substituted for conventional amino acids in solid phase peptide synthesis. However, production of polymers with reduced peptide bonds requires synthesis of the dimer of amino acids containing the reduced peptide bond. Such dimers are incorporated into polymers using standard solid phase synthesis procedures. Other synthesis procedures are well known in the art.

In one embodiment of the invention, an RNA interfering agent and the carrier polymer are combined together prior to contacting a biological membrane. Combining the RNA interfering agent and the carrier polymer results in an association of the agent and the carrier. In one embodiment, the RNA interfering agent and the carrier polymer are not indirectly linked together. Therefore, linkers are not required for the formation of the duplex. In another embodiment, the RNA interfering agent and the carrier polymer are bound together via electrostatic bonding.

It is known that depending upon the expression vector and transfection technique used, only a small fraction of cells may effectively uptake the siRNA molecule. In order to identify and select these cells, antibodies against a cellular target can be used to determine transfection efficiency through immunofluorescence. Preferred cellular targets include those which are present in the host cell type and whose expression is relatively constant, such as Lamin A/C. Alternatively, co-transfection with a plasmid containing a cellular marker, such as a CMV-driven EGFP-expression plasmid, luciferase, metalloprotease, BirA, β-galactosidase and the like may also be used to assess transfection efficiency. Cells which have been transfected with the siRNA molecules can then be identified by routine techniques such as immunofluorescence, phase contrast microscopy and fluorescence microscopy.

III. Methods of Treatment:

The present invention provides for both prophylactic and therapeutic methods of treating a subject having or at risk for, or susceptible to, apoptosis, inflammation, immune response, liver injury, or an apoptosis-mediated disease or disorder. As used herein, “treatment,” or “treating,” is defined as the application or administration of an interfering agent of the invention (e.g., an siRNA, e.g., an apoptosis-related gene siRNA or a proinflammatory cytokine siRNA) to a patient, or application or administration of a therapeutic agent to an isolated tissue or cell line from a patient, who has inflammation, immune response, liver injury, a cytokine mediated disease or disorder or an apoptosis-mediated disease or disorder, with the purpose to cure, heal, alleviate, relieve, alter, remedy, ameliorate, improve or affect the inflammation, immune response, liver injury, or an apoptosis-mediated disease or disorder, or symptoms of the apoptosis-mediated disease or disorder or cytokine mediated disease or disorder.

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with one or more RNA interfering agents, e.g., siRNAs or shRNAs, according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, an apoptosis-mediated disease or disorder or cytokine mediated disease or disorder, tissue injury, e.g., liver or T cell, hematopoietic cell, hepatocyte, neural cell and/or tumor cell injury caused by or related to apoptosis-related gene activity, e.g., apoptosis, or a proinflammatory cytokine activity, e.g., modulation of inflammation, or modulation of an immune response, by administering to the subject one or more therapeutic agents, e.g., the RNA interfering agents as described herein (e.g., one or more siRNA, e.g., an apoptosis-related gene, e.g., a Fas or FasL siRNA, or a proinflammatory cytokine, e.g., IL-1 or TNFα siRNA). Subjects at risk for an apoptosis-mediated disease or disorder or a proinflammatory cytokine disease or disorder, tissue injury, e.g., liver or T cell, hematopoietic cell, hepatocyte, neural cell and/or tumor cell injury caused by or related to apoptosis-related gene activity or a proinflammatory cytokine activity, e.g., Fas activity, inflammation, or an immune response, can be identified by, for example, any known risk factors for an apoptosis-mediated disease or disorder or a proinflammatory cytokine disease or disorder, tissue injury, e.g., liver or T cell, hematopoietic cell, hepatocyte, neural cell and/or tumor cell injury caused by or related to apoptosis-related gene activity, e.g., Fas activity, inflammation, or an immune response, e.g., hepatitis, e.g., alcohol induced hepatitis, or other forms of hepatitis, or subjects at risk for transplant rejection, cancer, or sepsis. Administration of a prophylactic agent can occur prior to the manifestation of symptoms characteristic of an apoptosis-mediated disease or disorder, a proinflammatory cytokine mediated disease or disorder, e.g., sepsis, tissue injury, e.g., liver or T cell, hematopoietic cell, hepatocyte, neural cell and/or tumor cell injury caused by or related apoptosis-related gene activity or a proinflammatory cytokine activity, e.g., Fas activity, inflammation, or an immune response, such that the apoptosis-mediated disease or disorder, tissue injury, inflammation, or immune response are prevented or, alternatively, delayed in their progression. In the case of transplantation, the transplanted organ or tissue, e.g., liver, may be treated with the RNA interfering agents of the invention prior to transplantation or the RNA interfering agent may be administered after transplantation, via any known method or any method described herein.

Any mode of administration of the therapeutic agents of the invention, as described herein or as known in the art, including topical administration of the siRNAs of the instant invention, may be utilized for the prophylactic treatment of an apoptosis mediated disease or disorder, a proinflammatory cytokine mediated disease or disorder, tissue injury, e.g., liver or T cell, hematopoietic cell, hepatocyte, neural cell and/or tumor cell injury caused by or related to apoptosis-related gene activity or a proinflammatory cytokine activity, e.g., Fas activity, inflammation, or an immune response.

2. Therapeutic Methods

Another aspect of the invention pertains to methods of modulating gene expression or protein activity, e.g., apoptosis-related gene expression, e.g., anti-apoptotic gene expression or pro-apoptotic gene expression, e.g., Fas gene expression, or protein activity or proinflammatory cytokine gene expression or protein activity, in order to treat an apoptosis-mediated disease or disorder or a proinflammatory cytokine mediated disease or disorder, tissue injury, inflammation, or immune response. Accordingly, in an exemplary embodiment, the modulatory method of the invention involves contacting a cell expressing an apoptosis-related gene, e.g., Fas, e.g., a T cell, hematopoietic cell, hepatocyte, neural cell and/or tumor cell, with one or more RNA interfering agent (e.g., an siRNA, e.g., an apoptosis-related gene siRNA, e.g., Fas or a proinflammatory cytokine gene siRNA, e.g., IL-1 or TNFα siRNA) that is specific for the target gene, e.g., an apoptosis-related gene, e.g., an anti-apoptotic gene or a pro-apoptotic gene, e.g., Fas, or a proinflammatory cytokine, e.g., IL-1 or TNFα such that expression of an apoptosis-related gene, e.g., an anti-apoptotic gene or a pro-apoptotic gene, e.g., Fas, or a proinflammatory cytokine, e.g., IL-1 or TNFα, is modulated. For example, an anti-apoptotic gene activity is inhibited in order to treat, for example, cancer; alternatively, a pro-apoptotic gene activity is inhibited in order to treat, for example, hepatitis, liver injury, or transplant rejection. These methods can be performed in vitro (e.g., by culturing the cell) or, alternatively, in vivo (e.g., by administering the agent to a subject).

One skilled in the art can readily determine the appropriate dose, schedule, and method of administration for the exact formulation of the composition being used, in order to achieve the desired “effective level” in the individual patient. One skilled in the art also can readily determine and use an appropriate indicator of the “effective level” of the compounds of the present invention by a direct (e.g., analytical chemical analysis) or indirect, or analysis of appropriate patient samples (e.g., blood and/or tissues).

The therapeutic compositions of the invention can also be administered to cells ex vivo, e.g., cells are removed from the subject, the compositions comprising the siRNAs or shRNAs of the invention are administered to the cells, and the cells are re-introduced into the subject. Vectors, e.g., gene therapy vectors, can be used to deliver the therapeutic agents to the cells. The cells may be re-introduced into the subject by, for example, intravenous injection.

The prophylactic or therapeutic pharmaceutical compositions of the invention can contain other pharmaceuticals, in conjunction with a vector according to the invention, when used to therapeutically treat or prevent an apoptosis-mediated disease or disorder or a proinflammatory cytokine mediated disease or disorder, tissue injury, inflammation, or immune response, and can also be administered in combination with other pharmaceuticals used to treat or prevent an apoptosis-mediated disease or disorder, or a proinflammatory cytokine mediated disease or disorder, tissue injury, inflammation, or immune response. For example, the prophylactic or therapeutic pharmaceutical compositions of the invention can also be used in combination with other pharmaceuticals which modulate the expression or activity of apoptosis-related genes, e.g., Fas or proinflammatory cytokines, e.g., IL-4.

Examples of pharmaceuticals used to treat or prevent apoptosis-mediated diseases or disorders tissue injury, e.g., liver or T cell, hematopoietic cell, hepatocyte, neural cell and/or tumor cell injury caused by or related to apoptosis-related gene activity, e.g., Fas activity, include, but are not limited to, treatment for hepatitis, e.g., immunosuppressive treatment with corticosteroids, recombinant interferon alpha, ribovirin, ursodeoxycholic acid, and vitamin E, as well as treatment with immunosuppressive agents for inhibition of transplant rejection, or other anti-inflammatory agents, cancer therapeutics, e.g., liver cancer therapeutics, and the like.

3. Pharmacogenomics

The RNA interfering agents as described herein (e.g., an siRNA, e.g., an apoptosis-related gene siRNA, e.g., a Fas siRNA or a cytokine siRNA) can be administered to individuals to treat (prophylactically or therapeutically) an apoptosis-mediated disease or disorder, or a proinflammatory cytokine mediated disease or disorder, tissue injury, inflammation, or immune response. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer one or more therapeutic RNA interfering agents as described herein (e.g., an siRNA, e.g., an apoptosis-related gene siRNA, e.g., a Fas siRNA, or a proinflammatory cytokine siRNA) as well as tailoring the dosage and/or therapeutic regimen of treatment with an RNA interfering agent, e.g., an siRNA, e.g., an apoptosis-related gene siRNA, e.g., a Fas siRNA or a proinflammatory cytokine siRNA.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug target is known, all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a therapeutic RNA interfering agent as described herein (e.g., an siRNA, e.g., an apoptosis-related gene siRNA, e.g., a Fas siRNA).

IV. Pharmaceutical Compositions

The RNA interfering agent, e.g., an siRNA of the invention can be incorporated into pharmaceutical compositions suitable for administration. Such compositions typically comprise the RNA interfering agent, e.g., an siRNA, such as an apoptosis-related gene siRNA, e.g., Fas siRNA, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Generally, the compositions of the instant invention are introduced by any standard means, with or without stabilizers, buffers, and the like, to form a pharmaceutical composition. For use of a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the like.

In one embodiment, the invention features the use of the compounds of the invention in a composition comprising surface-modified liposomes containing poly (ethylene glycol) lipids (PEG-modified, or long-circulating liposomes or stealth liposomes). In another embodiment, the invention features the use of compounds of the invention covalently attached to polyethylene glycol. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). The long-circulating compositions enhance the pharmacokinetics and pharmacodynamics of therapeutic compounds, such as DNA and RNA, particularly compared to conventional cationic liposomes which are known to accumulate in tissues of the MPS (Liu et al., J. Biol. Chem. 1995, 42, 2486424870; Choi et al., International PCT Publication No. WO 96/10391; Ansell et al., International PCT Publication No. WO 96/10390; Holland et al., International PCT Publication No. WO 96/10392). Long-circulating compositions are also likely to protect drugs from nuclease degradation to a greater extent compared to cationic liposomes, based on their ability to avoid accumulation in metabolically aggressive MPS tissues such as the liver and spleen.

Examples of routes of administration include parenteral, e.g., intravenous, intramuscular, intradermal, subcutaneous, oral (e.g., inhalation), transdermal (topical), transmucosal, vaginal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. The compounds can also be prepared in the form of suppositories (e.g., with conventional suppository bases such as cocoa butter and other glycerides) or retention enemas for rectal delivery.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to hepatocytes) can also be used as pharmaceutically acceptable carriers. These can be prepared according to methods known to those skilled in the art, for example, as described in U.S. Pat. No. 4,522,811 U.S. Pat. No. 5,643,599, the entire contents of which are incorporated herein.

Liposomal suspensions (including liposomes targeted to macrophages containing, for example, phosphatidylserine) can also be used as pharmaceutically acceptable carriers. These can be prepared according to methods known to those skilled in the art, for example, as described in U.S. Pat. No. 4,522,811 U.S. Pat. No. 5,643,599, the entire contents of which are incorporated herein. Alternatively, the therapeutic agents of the invention may be prepared by adding a poly-G tail to one or more ends of the siRNA for uptake into target cells. Moreover, siRNA may be fluoro-derivatized and delivered to the target cell as described by Capodici, et al. (2002) J. Immuno. 169(9):5196.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the siRNA in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an RNA interfering agent (i.e., an effective dosage) ranges from about 0.001 to 3,000 mg/kg body weight, preferably about 0.01 to 2500 mg/kg body weight, more preferably about 0.1 to 2000, about 0.1 to 1000 mg/kg body weight, 0.1 to 500 mg/kg body weight, 0.1 to 100 mg/kg body weight, 0.1 to 50 mg/kg body weight, 0.1 to 25 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an RNA interfering agent can include a single treatment or, preferably, can include a series of treatments.

In a preferred example, a subject is treated with an RNA interfering agent in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of RNA interfering agent used for treatment may increase or decrease over the course of a particular treatment. Changes in dosage may result and become apparent from the results of diagnostic assays as described herein.

It is understood that appropriate doses of the RNA interfering agents, e.g., siRNAs or shRNAs, depend upon a number of factors within the ken of the ordinarily skilled physician, veterinarian, or researcher. The dose(s) of the agent will vary, for example, depending upon the identity, size, and condition of the subject or sample being treated, further depending upon the route by which the composition is to be administered, if applicable, and the effect which the practitioner desires the agent, e.g., an siRNA to have upon the target gene, e.g., an apoptosis-related gene, e.g., the Fas gene.

The RNA interfering agents, e.g., siRNAs of the invention can be inserted into vectors. These constructs can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the vector can include the RNA interfering agent, e.g., the siRNA vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

This invention is further illustrated by the following examples which should not be construed as limiting. The contents of all references, patents and published patent applications cited throughout this application, as well as the Figures and Sequence Listing, are incorporated herein by reference.

### EXAMPLES

### Example 1

### RNA Interference Targeting FAS Inhibits FAS Expression In Vivo and Protects Mice from Fulminant Hepatitis and Fibrosis

RNA interference (RNAi) by synthetic 21-23 nucleotide siRNAs silences cellular and viral gene expression in mammalian cells in vitro (Elbashir, S. M. (2001) Nature 411, 494-8; Caplen, N. J., et al. (2001) Proc Natl Acad Sci USA 98, 9742-7). siRNA duplexes pulse-injected into the tail vein of mice inhibit co-transfected firefly luciferase gene expression (McCaffrey, A. P. et al. (2002) Nature 418, 38-9; Lewis, D. L., et al. (2002) Nat Genet 32, 107-8). Viral-mediated delivery of siRNA in vivo reduces exogenous GFP and endogenous glucuronidase expression (Xia, H. et al. (2002) Nat Biotechnol 20, 1006-10). In these studies, RNA silencing was prominent in the liver, indicating that the liver is an ideal organ to test the therapeutic potential of siRNA. Hepatocytes are very susceptible to Fas-mediated apoptosis because they highly express Fas (Kondo, T., et al. (1997) Nat Med 3, 409-13). As a consequence, Fas-mediated apoptosis plays an important role in hepatic injury from diverse insults, including viruses, autoimmunity and transplant rejection (Rust, C. & Gores, G. J. (2000) Am J Med 108, 567-74; Siegel, R. M. & Fleisher, T. A. (1999) J Allergy Clin Immunol 103, 729-38.). Fas-deficient lpr mice survive challenge with factors that induce fulminant hepatitis (Ogasawara, J. et al. (1993) Nature 364, 806-9; Li, X. K. et al. (2001) Transplantation 71, 503-8) and have reduced fibrosis after chronic hepatic insults (Canbay, A. et al. (2002) Gastroenterology 123, 1323-30). Thus, whether intravenous siRNA injection targeting Fas could inhibit Fas expression on murine hepatocytes in vivo and protect the liver from fulminant hepatitis and fibrosis is investigated.

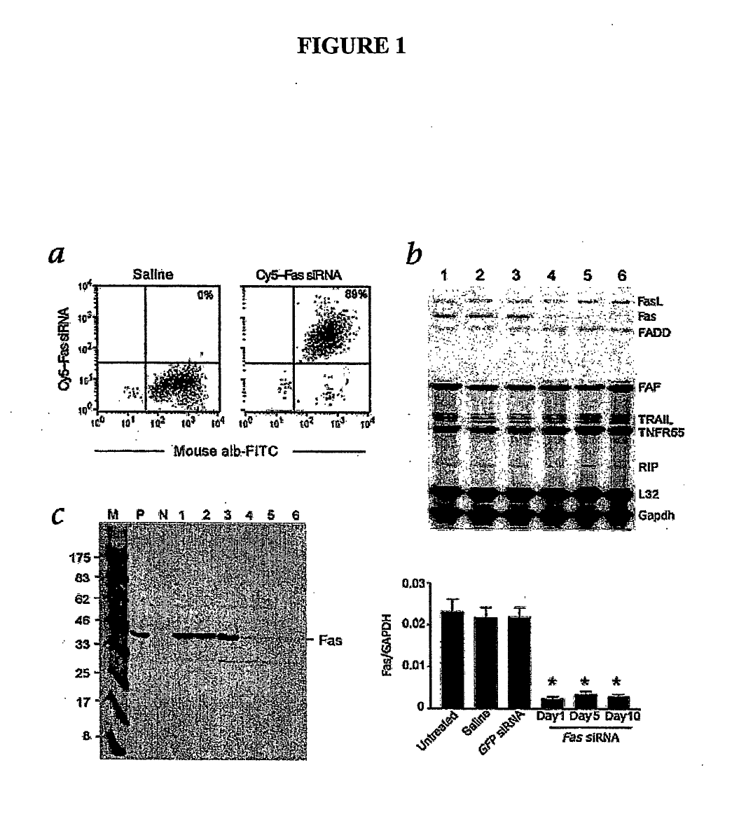

First, delivery of synthetic siRNA duplexes into mouse hepatocytes in vivo by hydrodynamic tail vein injection of Cy5-labeled Fas (sequence 1)-siRNA (50 μg, 2.0-2.5 mg/kg) (Zhang, G., et al. (1999) Hum Gene Ther 10, 1735-7) was verified. Twenty-four hours after the last of three injections, 88±6% of hepatocytes had taken up the siRNA and were Cy5⁺ by flow cytometry (FIG. 1a). The efficient delivery confirms that siRNA duplexes can be taken up by most liver cells in vivo (McCaffrey, A. P. et al (2002) Nature 418, 38-9; Lewis, D. L., (2002) Nat Genet 32, 107-8). Transduction is higher than the 40% efficiency observed with a single injection of reporter plasmid DNA (Zhang, G. (1999) Hum Gene Ther 10, 1735-7). Fas mRNA and protein expression in hepatocytes was measured by RNase protection assay (RPA) and immunoblot, respectively, at various times following injection. Treatment with Fas (sequence 1)-siRNA reduced Fas mRNA expression 8-10 fold compared to saline or GFP-siRNA injection (fas/GAPDH signal: 0.0024±0.0004 vs. 0.022±0.002 saline or 0.022±0.002 GFP-siRNA n=3/group, P<0.001 compared with either control) 24 hours after the last injection (FIG. 1b). In parallel, immunoblot analysis also demonstrated that Fas (sequence 1)-siRNA reduced Fas protein in hepatocytes nearly to background (FIG. 1c). The effect was specific, as injection of control siRNA targeting GFP did not change Fas expression, and Fas (sequence 1)-siRNA treatment did not affect the expression of other Fas related genes, such as FasL, FADD, FAF, TRAIL, and TNF receptor p55 (FIG. 1b). Specificity was also demonstrated by RPA of hepatocytes from mice injected with siRNAs targeting other regions of fas. Two additional siRNAs, sequences 5 and 6 silenced fas expression by ˜81-86%, as efficiently as sequence 1, while sequence 2, beginning only one nucleotide downstream of sequence 6, only reduced fas expression by 38% (FIG. 4e). Two sequences (sequences 3 and 4) did not suppress fas expression at all. siRNA apparently suppresses expression more efficiently than intraperitoneal injection of antisense oligodeoxynucleotide (ODN). In a previous report, mice were treated with a higher dosage (6 mg/kg) of anti-fas ODN for 12 consecutive days. Approximately 14-fold less total nucleic acid was administered here (50 ug for 3 injections to mice weighing approximately 24 grams) to reduce Fas expression similarly. In a recent study, siRNA was quantitatively more efficient than antisense ODN at suppressing co-transfected GFP expression both in vitro and in vivo (Zhang, H. et al. (2000) Nat Biotechnol 18, 862-7; Bertrand, J. et al. (2002) Biochem Biophys Res Commun 296, 1000).

A major concern of applying siRNA therapeutically is the stability of silencing under physiological conditions. Double-stranded siRNAs resist biodegradation in fetal calf serum and in human plasma (Bertrand, J. et al. (2002) Biochem Biophys Res Commun 296, 1000), and in one study siRNA-directed suppression of a co-transfected gene in vivo in mouse liver was maintained for several days (Lewis, D. L., et al. (2002) Nat Genet 32, 107-8). In this study, both mRNA and protein Fas levels were stably reduced for 10 days after the last injection (fas/GAPDH signal day 1: 0.0024±0.0004, day 5: 0.0035±0.0006, day 10: 0.0029±0.0004, P>0.05) (FIGS. 1b and 1c). Fas mRNA and protein expression was still only 40% of that in control animals on day 14, but returned to normal 20 days after the last injection.

While not intending to be bound by theory, the duration of silencing in hepatocytes, which contrasts with more transient silencing in transformed cell lines (Elbashir, S. M. (2001) Nature 411, 494-8), suggests that sustained therapeutic silencing in hepatocytes does not require siRNA expression from plasmids or viral vectors. The difference between liver cells and cell lines is likely because hepatocytes are mostly nondividing, so there is no siRNA dilution with cell division. However, the possibility that sustained suppression in hepatocytes might be due to siRNA amplification, which occurs in lower species (Ketting, R. F. et al. (2001) Genes Dev 15, 2654-9; Lipardi, C., (2001) Cell 107, 297-307) (Schwarz, D. S., (2002) Mol Cell 10, 537-48), is not ruled out. Because silencing after duplex siRNA injection is prolonged but not permanent, long-term toxicity, such as lymphoproliferative or autoimmune disease, seen in humans with mutations of fas and in the lpr mouse (Takahashi, T. et al. (1994) Cell 76, 969-76), is of no concern.

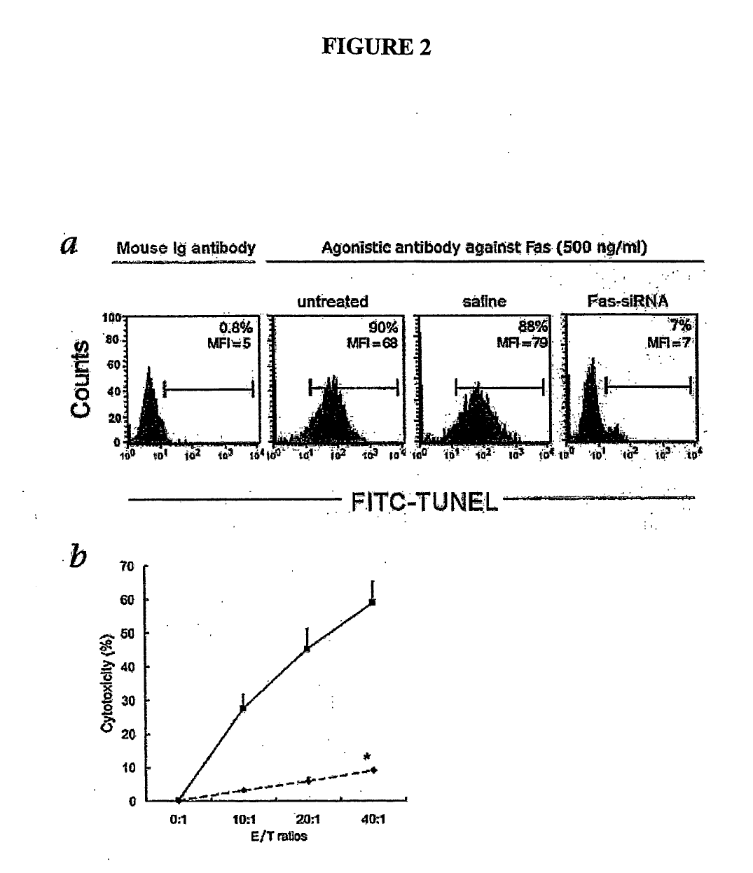

Two known mechanisms underlie Fas-mediated fulminant hepatitis. Ligation of the Fas receptor on hepatocytes induces massive apoptosis, accompanied by an infiltration of inflammatory cells and secondary necrosis (Ryo, K. et al. (2000) Am J Gastroenterol 95, 2047-55). Fas engagement also provokes hepatic inflammation by inducing expression of hepatic chemokines that recruit and activate immune cells, leading to hepatocyte death in a proinflammatory milieu (Faouzi, S. et al. (2001) J Biol Chem 276, 49077-82). To determine whether the efficient suppression of Fas expression in the liver after duplex siRNA injection protects hepatocytes from Fas-mediated apoptosis, hepatocytes from Fas(sequence1)-siRNA treated or mock-injected mice were challenged in vitro with agonistic anti-Fas antibody (Jo2) or activated hepatic mononuclear cells (MNCs) harvested from conA-treated mice. In vitro exposure of hepatocytes from untreated or mock-treated mice to Jo2 antibody (500 ng/mL) for 24 hours resulted in 87.8±6.8% (n=5) apoptotic cells measured by FITC-TUNEL staining (FIG. 2a). In contrast, only 7.9±1.5% (n=5) of cultured hepatocytes from Fas (sequence1)-siRNA treated mice stained with TUNEL (P<0.0001). Moreover, protection from in vitro fas-induced apoptosis after tail vein injection of other fas-siRNAs correlated with fas suppression; fas-siRNA that did not silence had no effect on apoptosis, while only partial protection was provided by partial silencing. A recent study shows that FasL-expressing natural killer T (NKT) cells are the hepatic mononuclear cells (MNCs) that induce hepatic cell injury in ConA-induced hepatitis (Seino, K. et al. (1997) Gastroenterology 113, 1315-22; Takeda, K. et al. (2000) Proc. Natl. Acad. Sci., U.S.A. 97, 5498-503). Therefore it was tested by ALT release assay whether mouse hepatocytes from Fas (sequence1)-siRNA treated mice were resistant to cytolysis by hepatic MNCs isolated from ConA-treated mice (FIG. 2b). Hepatocytes from Fas-siRNA treated mice were not lysed by hepatic MNCs, while hepatocytes from mock-treated or untreated controls were. Therefore, Fas silencing effectively inhibits hepatocyte apoptosis in vitro.

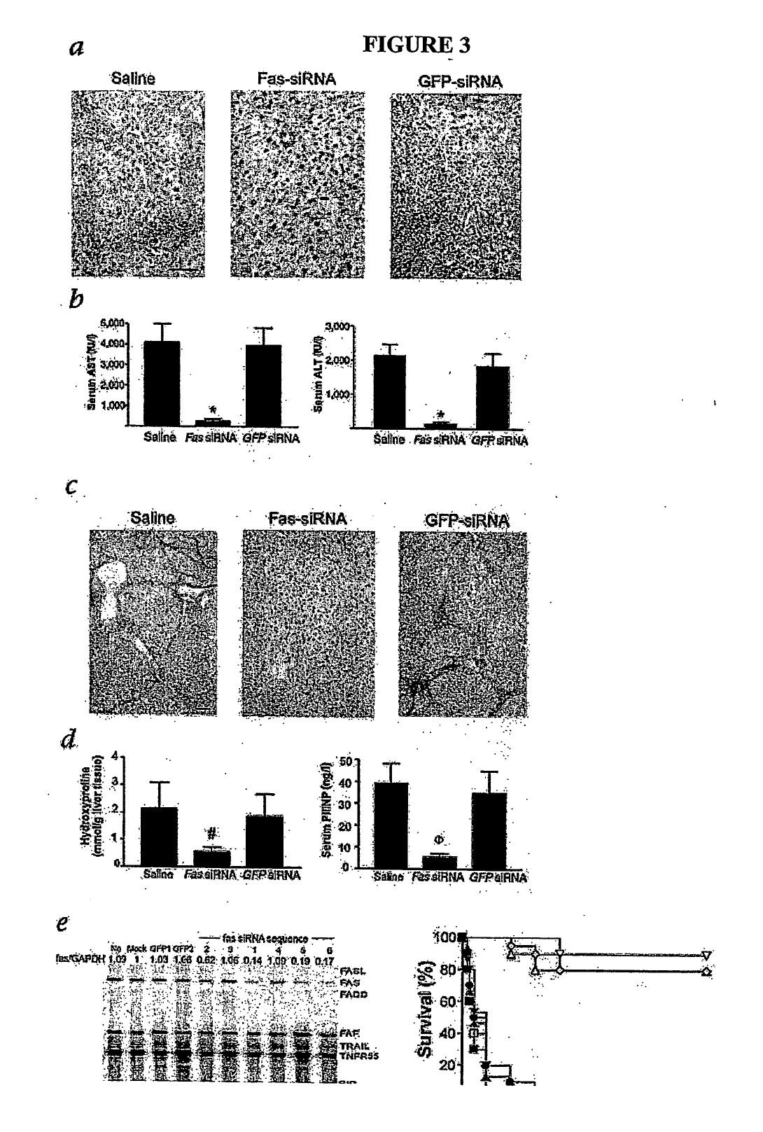

Whether Fas-siRNA treatment protects mice from fulminant hepatitis in two models of Fas-mediated liver damage was also examined. Mice treated with Fas (sequence1)-siRNA, GFP (sequence1)-siRNA or saline were challenged one day later by intravenous injection of conA. Serum transaminase levels and liver pathology were analyzed 20 hours after conA challenge. All control saline and GFP-siRNA-treated mice had extensive liver damage with confluent hepatocyte necrosis with bridging and inflammatory cell infiltrates surrounding the portal and central veins (FIG. 3a). Most surviving hepatocytes had cytoplasmic swelling, and there was frequent nuclear chromatin condensation, indicative of apoptosis. In contrast, pretreatment with Fas-siRNA prevented liver cell necrosis and abrogated inflammatory infiltration, although mild hepatocyte swelling was detected. In conA-induced hepatitis, release of transaminases alanine aminotransferase (ALT) and aspartate aminotransferase (AST) from damaged hepatocytes peaks in the serum 20 hours after injection and is a good indicator of the extent of liver damage (Miyazawa, Y., et al. (1998) Hepatology 27, 497-506). In agreement with the morphological findings, Fas-siRNA treatment almost completely prevented the elevation of serum ALT (142±58 IU/L vs. 2150±312 IU/L in saline-treated controls; n=5, P<0.001 (normal, 30 IU/L)) and AST (270±90 IU/L vs. 4120±876 IU/L in saline-treated controls; n=5, P<0.001 (normal, 50 IU/L)) (FIG. 3b).

Fas-mediated hepatocyte apoptosis also contributes to the development of liver fibrosis in chronic hepatitis (Canbay, A. et al. (2002) Gastroenterology 123, 1323-30; Galle, P. R. et al. (1995) J Exp Med 182, 1223-30). To evaluate further the therapeutic potential of Fas-siRNA to treat chronic liver injury and to determine whether siRNA administered after the noxious insult can protect in a more clinically relevant scenario, siRNA treatment was delayed until 24 hours after the second of six weekly injections with a reduced dose of ConA. The siRNA injection was repeated once two weeks later. Mice were sacrificed at seven weeks, one week after the last ConA injection. All mock and GFP-siRNA-treated mice developed bridging fibrosis in the liver parenchyma, while there was no hepatic fibrosis or necrosis in Fas (sequence1)-siRNA-treated animals (FIG. 3c). Additionally, Fas-siRNA treatment significantly reduced two chemical indicators of active fibrosis, hepatic hydroxyproline (Takahashi, S. & Lee, M. J. (1987) Biochem J 241, 49-54) (0.56±0.17 mmol/g liver tissue in Fas-siRNA-treated mice vs. 2.13±0.95 mmol/g in mock-treated controls; n=3, P<0.05; normal, 0.5 mmol/g) and serum procollagen type III (PIIINP) (Badawy, A. A. et al. (1996) Pharmacol Res 33, 319-25) (5.6±1.5 ng/L in Fas-siRNA-treated mice vs. 39.2±2.1 ng/L in saline-treated controls; n=3, P<0.01; normal 5 ng/L) (FIG. 3d). Furthermore, there was no evidence of toxicity, including lymphoproliferation, splenomegaly or other organ damage, from prolonged silencing of fas, even with repeated injection. These results suggest that Fas-siRNA treatment provides protection even after the initiation of chronic liver injury.

To evaluate further whether Fas-siRNA promotes survival in fulminant hepatitis, mice were challenged in a more aggressive hepatitis model (Ogasawara, J. et al. (1993) Nature 364, 806-9) by intraperitoneal injection of Fas antibody. All control animals (n=40) died within 3 days, mostly within 24 hours after antibody injection. Moreover, mice treated with fas-siRNAs (sequences 2, 4), which only silenced expression by 38%, also were not protected. However, mice pretreated with Fas-siRNAs that silenced expression by 81-86% (sequences 1, 5, 6) were protected from lethal challenge with 33 of 40 animals surviving for the 10 days of observation (logrank test, P<0.0001) (FIG. 3e). Fatalities in the Fas-siRNA-treated group were from hemorrhage secondary to liver failure. Liver damage in lethal fulminant hepatitis culminates within the first few weeks, but the survivors recover thereafter (Dhiman, R. K., et al. (1998) Dig Dis Sci 43, 1311-6). In fact, livers and other organs from the surviving mice appeared normal when sacrificed at the end of the observation period. Hence, Fas silencing during the acute insult prevents death from fulminant hepatitis.

Based on the crucial role that Fas-mediated apoptosis plays in a broad spectrum of immune-related liver diseases, siRNA-directed Fas silencing may be of therapeutic value to prevent and treat acute and chronic liver injury induced by viral and autoimmune hepatitis Rust, C. & Gores, G. J. (2000) Am J Med 108, 567-74; Siegel, R. M. & Fleisher, T. A. (1999) J Allergy Clin Immunol 103, 729-38) alcoholic liver disease, acute and chronic liver failure (Ryo, K. et al. (2000) Am J Gastroenterol 95, 2047-55; Galle, P. R. et al. (1995) J Exp Med 182, 1223-30), and rejection of liver transplantation (Rust, C. & Gores, G. J. (2000) Am J Med 108, 567-74). Furthermore, while hydrodynamic injection may be difficult in humans, regional delivery of high concentrations of siRNA via hepatic artery or portal vein cannulation is a viable alternative.

Methods

Preparation of siRNAs. siRNAs were synthesized using 2′-O-ACE-RNA phosphoramidites (Dharmacon Research™, Lafayette, Colo.). The sense and anti-sense strands of siRNAs are:

Fas sequence 1 beginning at nt 364:

(SEQ ID NO:1)

| | |
|---|---|
| 5′-P.GUGCAAGUGCAAACCAGACdTdT-3′ | (sense), |

(SEQ ID NO:2)

| | |
|---|---|
| 5′-P.GUCUGGUUUGCACUUGCACdTdT-3′ | (antisense); |
| Fas sequence 2 beginning at nt 874: | |

(SEQ ID NO:3)

| | |
|---|---|
| 5′-P.AGCCGAAUGUCGCAGAACCdTdT-3′ | (sense); |

(SEQ ID NO:4)

| | |
|---|---|
| 5′-P.GGUUCUGCGACAUUCGGCUdTdT-3′ | (antisense); |
| Fas sequence 3 beginning at nt 137: | |

(SEQ ID NO:5)

| | |
|---|---|
| 5′-P.GGAUUAUAUCAAGGAGGCCdTdT-3′ | (sense); |

(SEQ ID NO:6)

| | |
|---|---|
| 5′-P.GGCCUCCUUGAUAUAAUCCdTdT-3′ | (antisense); |
| Fas sequence 4 beginning at nt 501: | |

(SEQ ID NO:7)

| | |
|---|---|
| 5′-P.AUCGCCUAUGGUUGUUGACdTdT-3′ | (sense); |

(SEQ ID NO:8)

| | |
|---|---|
| 5′-PGUCAACAACCAUAGGCGAUdTdT-3′ | (antisense); |
| Fas sequence 5 beginning at nt 667: | |

(SEQ ID NO:9)

| | |
|---|---|
| 5′-P.AUACAUCCCGAGAAUUGCUdTdT-3′ | (sense); |

(SEQ ID NO:10)

| | |
|---|---|
| 5′-P.AGCAAUUCUCGGGAUGUAUdTdT-3′ | (antisense); |
| Fas sequence 6 beginning at nt 873: | |

(SEQ ID NO:11)

| | |
|---|---|
| 5′-P.AAGCCGAAUGUCGCAGAACdTdT-3′ | (sense); |

(SEQ ID NO:12)

| | |
|---|---|
| 5′-P.GUUCUGCGACAUUCGGCUUdTdT | (antisense); |
| GFP sequence 1: | |

(SEQ ID NO:13)

| | |
|---|---|
| 5′-P.GGCUACGUCCAGGAGCGCACC-3′ | (sense), |

(SEQ ID NO:14)

| | |
|---|---|
| 5′-P.UGCGCUCCUGGACGUAGCCUU-3′ | (antisense); |
| GFP sequence 2: | |

(SEQ ID NO:15)

| | |
|---|---|
| 5′-P.GGCUACGUCCAGGAGCGCAdTdT-3′ | (sense), |

(SEQ ID NO:16)

| | |
|---|---|
| 5′-P.UGCGCUCCUGGACGUAGCCdTdT-3′ | (antisense). |

RNAs were deprotected and annealed according to the manufacturer's instruction. Cy5-labeled Fas-siRNA with the fluorophore coupled to the 3′ end of the sense strand was produced by Dharmacon Research™.

siRNA treatment. Male BALB/c mice, 8-10 weeks of age weighing 20-25 g, were purchased from Jackson Laboratory (Bar Harbor, Me.). Synthetic siRNAs were delivered in vivo using a modified “hydrodynamic transfection method (Zhang, G., et al. (1999) Hum. Gene Ther. 10:1735-7), by which 50 μg siRNA dissolved in 1 ml PBS was rapidly injected into the tail vein. The injection was repeated 8 and 24 hours later. Control mice were injected with an equal volume of normal saline or GFP-siRNA.

Isolation of hepatocytes. Hepatocytes were isolated by modified hepatic portal perfusion technique (Klauning, J. E., et al. (1981) In vitro 17:913-925). The purity of hepatocytes, determined by flow cytometric analysis of intracellular albumin staining using fluorescein-conjugated goat anti-mouse albumin antibody (Bethyl Laboratories, Inc., Montgomery, Tex.), was >90% (not shown). For some experiments, cells were briefly cultured after plating at 2×10⁶ cells/60 mm collagen-coated culture dishes in William's Medium E (Gibco BRL, Grand Island, N.Y.) supplemented with 10% fetal bovine serum, 15 mmol/L HEPES (pH 7.4), 1 umol/L insulin, 2 mmol/L L-glutamine, 100 units/mL penicillin, and 100 ug/mL streptomycin.

RNase protection assay. Total RNA was extracted from hepatocytes using Trizol reagent (Molecular Research Center, Cincinnati, Ohio), and RNase protection assay (RPA) was performed using 15 μg of total RNA and the In-vitro Transcription Kit and mouse mAPO-3 multi-probe template set (BD Pharmingen, San Diego, Calif.) according to the manufacturer's instructions. Intensities of the protected bands were quantified by phosphorimaging (Fuji-BAS 1500; Fuji, Tokyo, Japan) based on the ratios of the investigated genes to GAPDH (internal control).

Immunoblot. Protein extracts of mouse hepatocytes were resolved over 12% SDS-polyacrylamide gels, transferred onto nitrocellulose membranes, probed with rabbit polyclonal anti-mouse Fas antibody and peroxidase-conjugated goat anti-rabbit secondary antibody (Oncogene Research Product, Boston, Mass.) and visualized by chemiluminescence (Amersham Life Science, Arlington Heights, Ill.).

Apoptosis assay. Primary hepatocytes from untreated mice or mice treated with Fas-siRNA or saline were seeded in 12-well plates at a density of 1×10⁵/mL. The next day Jo2 mAb (500 ng/mL; BD PharMingen, San Diego, Calif.) was added and 24 hours later, liver cell apoptosis was evaluated by FITC-labeled terminal deoxynucleotide transferase-mediated dUTP nick end labeling (TUNEL) assay (Boehringer Mannheim GmbH, Mannheim, Germany) analyzed by flow cytometry on a FACScan flow cytometer with LYSIS II software (Nippon Becton Dickinson, Tokyo, Japan).

ALT release assay. Target liver cells, plated in 12-well plates at 1×10⁴ cells/well, were co-cultivated overnight with hepatic mononuclear cells (MNCs) isolated from livers of conA (15 mg/kg)-treated mice at indicated effector-to-target ratios. Release of ALT from hepatocytes was measured in the supernatants using an ALT assay kit (Boehringer Mannheim GmbH, Mannheim, Germany). Cytotoxicity was expressed as the percentage of ALT in the supernatants, compared to total ALT in detergent lysed cells.

Induction of hepatitis. Mice were injected intravenously through the tail vein with conA (15 mg/kg; Sigma, St. Louis, Mo.) reconstituted in pyrogen-free saline (Takeda, K., et al. (2000) Proc. Natl. Acad. Sci., USA 97:5498-503). After 20 hours, serum ALT and AST were measured using a standard clinical automatic analyzer (Hitachi, type 7150, Tokyo, Japan), and paraffin-embedded liver sections were stained with hematoxylin/eosin (HE). Other mice were injected intraperitoneally with 8 μg of Jo2 mAb and followed for 10 days before sacrifice and examination of liver histology.

Induction of liver fibrosis. A reduced dose of ConA (8 mg/kg) dissolved in pyrogen-free saline was injected in the tail vein weekly for 6 consecutive weeks (Kimura, K. et al Int Immunol 11, 1491-500 (1999)). Mice were treated with three injections of siRNA as above beginning 24 hours after the second and fourth Con A injections and sacrificed 1 week after the final injection of ConA. Liver fibrosis was evaluated by HE staining and measurement of hepatic hydroxyproline (Takahashi, S. & Lee, M. J. Collagen Biochem J 241, 49-54 (1987)) and serum procollagen type III using a sequential saturation radioimmunoassay (Calbiochem, La Jolla, Calif.), as described (Badawy, A. A. et al. Evaluation of colchicine with or without praziquantel therapy in the control of hepatic fibrosis in murine schistosomiasis. Pharmacol Res 33, 319-25 (1996))

### Example 2

### Identification of Efficient Method for Transduction of Cell Types and Tissues

This example compares the ability of RNA interfering agents delivered by hydrodynamic injection, or by injection of plasmid DNA, retrovirus or lentivirus encoding hairpin siRNA to establish a silenced state in different cell types and tissues in vivo. The extent of silencing is also compared with that achieved when siRNAs are expressed from a hairpin transgene (either constitutively or inducibly via Cre-recombinase in certain tissues) in transgenic mice which globally express a reporter gene (eGFP) in all cells. All of the constructs required to silence GFP expression have been produced and shown to effectively silence GFP expression in a variety of primary cells (macrophages, dendritic cells, T lymphocytes) in vitro. For each delivery method, the delivery protocol for GFP silencing is optimized in groups of 3-5 adult transgenic-GFP mice which globally express GFP. Control mice are treated with irrelevant duplex siRNAs or with empty vectors or with vectors expressing irrelevant (i.e. CCR5) hairpin siRNA. The analysis of the effectiveness of GFP silencing is by fluorescence microscopy, flow cytometry, immunoblot, Northern blot and modified Northern blot of whole mouse sections and isolated specific cell populations as described above.

Hydrodynamic Injection

Duplex siRNAs are particularly attractive for inducing gene silencing, e.g., for bioterrorism application, because the effects of silencing are likely to be short-lived without long-term toxicity, unlike vectors which can integrate into the host genome and therefore can be expressed for the life of the cell or potentially cause cellular transformation by integration in an unfortuitous location.

Transduction may vary significantly in tissues and cells and will be most efficient in highly vascularized organs such as the liver and lung and that cells that are easy to transfect (myocytes, phagocytic cells (such as tissue macrophages) and hepatocytes) will be efficiently transduced, while cells that are more difficult to transfect (such as lymphocytes) are not transduced in vivo by hydrodynamic injection. In experiments to determine which tissues are easily transduced the same protocol is used as was used for fas-siRNA to inject fluorescently labeled, duplex GFP-siRNA bearing Cy5 coupled to the 5′-end of the sense strand to detect transduced cells, as described above. Modifications of the sense strand do not affect the induction of the silenced state. Comparison of Cy5 and GFP fluorescence 24 hours after the last injection enables identification the cells and tissues that are most readily transduced, quantification of the level of transduction and silencing by measuring mean fluorescence intensities and determination of whether all transduced cells are able to establish the silenced state. Changes in the efficiency of silencing are examined after independently varying the concentration of siRNA injected, the volume injected, the speed of injection, the number of injections and the time after the last injection. Focus is especially on whether these changes affect the transduction of hepatocytes and macrophages and determine whether the injection volume can be reduced substantially without jeopardizing transduction since the large volume injected (relative to the mouse blood volume) may limit applicability to humans.

Although hepatocytes can be efficiently transduced by hydrodynamic injection, macrophages may be more difficult to transduce. Several strategies are investigated for specifically targeting macrophages, taking advantage of the unique properties of macrophages, which are constantly sampling the environment and have special receptors for uptake of anionic polymers and phosphatidyl serine (PS) on apoptotic cells. These strategies are tested in vitro using MDMs prepared by culturing adherent PBMCs with GM-CSF as described.

If activated MDMs are transduced by any of these strategies in vitro, the transduction of MDMs is compared with that of freshly isolated adherent PBMCs and with LPS-activated macrophages to determine how macrophage activation affects the transfection efficiency and silencing in vitro. The efficiency of transduction is analyzed using flow cytometry to detect and quantify the MFI of incorporated Cy5-labeled siRNAs. The efficiency of uptake of soluble siRNA is compared with that of siRNA packaged into liposomes, prepared as described, using different ratios of lipid to siRNA. Liposome composition is also modified to incorporate varying concentrations of PS, which should enhance uptake via the macrophage PS receptor used for the recognition and phagocytosis of apoptotic cells (Fadok V A, et al. (2000) Nature , 405, 85-90; Fadok V A, and Chimini G. (2001) Semin Immunol, 13, 365-372; Hoffmann P R, et al. (2001) J Cell Biol, 155, 649-659; Huynh M L, et al. (2002) J Clin Invest, 109, 41-50).

To produce liposomes, phospholipids in chloroform/methanol (90:10) are dried under nitrogen gas, resuspended in PBS containing various concentrations of duplex Cy5-Iabeled siRNA and sonicated for 3′ at 4° C. The liposomes are added to plated MDMs (−10⁵ cells/well) using approximately 1 uM lipid/well with rocking for 1 hour (Huynh M L, Fadok V A, and Henson P M. (2002) J Clin Invest, 109, 41-50). The transfection efficiency is determined after overnight culture and washing by epifluorescence microscopy and quantitated by flow cytometry. The transfection conditions are optimized as to the ratio of PS/phosphatidyl choline (PC), the ratio of lipid/siRNA and the amount of lipid added/10⁵ cells. For the later purpose of silencing proinflammatory cytokines, the use of PS liposomes is particularly suitable. This is because macrophage engulfinent via the PS receptor promotes an anti-inflammatory response by increasing TGF-β1 secretion (Huynh M L, et al. (2002) J Clin Invest, 109, 41-50). Therefore if the macrophages are successfully transfected, not only will the proinflammatory cytokines be silenced, but the macrophage will also be induced to secrete anti-inflammatory cytokines.

The optimal liposomal preparations in vitro are then tested by tail vein hydrodynamic injection into mice. Since uptake and silencing function may not correlate (particularly if siRNAs are unstable in endocytic vesicles), uptake measured by Cy5 fluorescence is compared with GFP silencing for the most attractive delivery methods. The best delivery methods are then be tested in vivo by tail vein injection and optimized as described above. If in vitro studies suggest that the state of macrophage activation has an important effect on transfection or silencing efficiencies, the in vivo silencing induced by local instillation of liposomes into uninflamed and inflamed tissues is also compared.

As a model of uninflamed tissue, endotracheal instillation is used as described (Huynh, M. L. et al. (2002) J. Cell Biol. 155, 649). As a model for inflamed tissue, one ml of 1% thioglycollate broth is injected intraperitoneally and liposomes are injected 3 days later. Macrophages are harvested from the peritoneal cavity 1 day later via injecting and withdrawing PBS.

Another modification tested is adding a polyG tail 5-10 nucleotides in length to the 5′ end of the sense strand of the siRNA, which should enhance uptake via the macrophage scavenger receptor (Srividya S, et al. (2000) Biochem Biophys Res Commun, 268, 772-777). Another approach is to couple an activated thiol at the 5′ end of the sense stand to a cysteine residue added to the C terminal of on one of the cationic peptides that deliver molecules into the cytosol (such as residues 49-57 of tat or residues 43-58 of the Drosophila Antennapedia peptide) (Prochiantz A. (1996) Curr Opin Neurobiol, 6, 629-634; Moy P, et al. (1996) Molecular Biotechnology, 6, 105-113; Kim D T, et al. (1997) J Immunol, 159,1666-1668; Suzuki T, et al. (2002) J Biol Chem, 277, 2437-2443. Internalization via these peptides bypasses the endocytic pathway and therefore removes the danger of rapid degradation in the harsh lysosomal environment. Moreover, for antisense oligonucleotides, coupling to the Antennapedia peptide reduces the concentration required for biological efficacy by more than 1,000-fold compared to free oligonucleotide (Allinquant B, et al. (1995) J Cell Biol, 128, 919-927). Because coupling to the 5′ end will preclude using Cy5-labeled siRNA, for these approaches GFP silencing in mouse MDM derived from Tg-GFP mice is used as an indicator of effective delivery. In addition, covalent coupling of the siRNA to the tat peptide may be unnecessary for internalization (Sandgren S, et al. (2002) J Biol Chem, 277, 38877-38883). Because the basic peptide binds tightly to the acidic siRNA, it is also investigated whether mixtures of tat peptide and Cy5-siRNA (preincubated for 30′ at RT at various ratios) are internalized into MDMs. If this is effective in vitro, the ability of tat-siRNA mixtures to transduce lung macrophages in vivo will be tested following endotracheal instillation or i.p. injection into thioglycollate-treated mice.

In addition, it may be possible to enhance in vivo transfection of all cells by chemical modification of the duplex siRNA. Naked siRNA, produced with fluorine-derivatized cytidine 5^{J}-triphosphate and UTP to make it resistant to RNaseA, was able to silence luciferase and HIV gag expression in vitro even in activated primary T lymphocytes in the presence of serum (Capodici J, Kariko K, and Weissman D. (2002) J Immunol, 169, 5196-5201). Therefore if the duplex RNA can be synthesized to resist degradation, it may efficiently get into cells without a transfection agent.

Vectored Delivery of siRNA Encoded by Hairpin Constructs

For use of RNAi as an acute maneuver in the face of recent or imminent infection, short-term exposure to exogenous siRNA may be preferable to the longer term expression provided by viral vectors. This strategic decision is based partly on concerns about the disruption of genes and potential for malignant transformation following lentiviral and retroviral insertion (Bushman F D. (2002) Curr Top Microbiol Immunol, 261,165-177). However, use of viral vectors is desirable in some situations.

Lentiviruses expressing small hairpins that target HIV gene expression have been produced and used to silence HIV replication in cell lines. Recent studies have shown fairly efficient transduction of self-inactivating HIV-based lentiviral vectors in the liver (8-30%) and spleen (24%) (Naldini L, (1996) Science, 272, 263-267; Vanden Driessche T, (2002) Blood, 100, 813-822; Follenzi A, et al. (2002) Hum Gene Ther, 13, 243-260). Both hepatocytes and macrophages, which are targets described herein, were efficiently-transduced when mice were injected with high titer virus (produced in 293T cells by cotransfection with Rev and RRE helper plasmids and a VSV-envelope-plasmid) in the presence of 40 ng/ml polybrene as described. Although B lymphocytes were efficiently transduced, T lymphocytes were not.

A recent strategy using a lentiviral env that encodes a single chain antibody that binds to and activates the T cell receptor complex may be useful to infect T lymphocytes (Maurice M, (2002) Blood, 99, 2342-2350). This vector may be used to express fasL-shRNA to prevent fulminant hepatitis, if silencing fasL is preferable to silencing fas in transgenic mice studies. The best hydrodynamic approaches for transducing hepatocyte and macrophages with in vivo delivery of injected plasmid DNA, retrovirus (pBpU6-sIGFPI) and lentivirus (pRRLU6GFP1sin) are compared. The analysis of the effectiveness of GFP silencing is by fluorescence microscopy, flow cytometry, immunoblot, Northern blot and modified Northern blot of whole mouse sections and isolated specific cell populations (liver, spleen, hepatocytes, macrophages, B and T lymphocytes).

### Example 3

### Silencing Proinflammatory Cytokine Gene Expression in Macrophages

An siRNA sequence is chosen by identifying an “AA” followed by 19 nucleotides, and then, if possible, a “TT” is identified (the last feature is optional). The candidate sequence may have a GC content of about 35% to about 70% and be at least 75 nucleotides downstream from the ATG start site. A final requirement is that the sequence lack homology with other genes. Homology can be determined using a Blast search using an libraries, including the est sequence database. This algorithm is used to choose 3 or 4 sequences for each targeted cytokine gene (TNFα, IL-1) and have duplex siRNAs synthesized (Dharmacon). The proinflammatory cytokine IL-6 is not targeted, since in some rodent models IL-6 production is protective and blocking it leads to more lethality during septic shock (Barton B E, and Jackson J V (1993) Infect Immun, 61, 1496-1499). Each sequence is tested by transfection with oligofectamine into MDMs using the methods which have already been optimized for silencing HIV infection. One day after transfection the MDMs are activated with LPS and tested the next day by intracellular cytokine staining (ICC) and Western blot and by Northern blot and RNase protection assay for protein and mRNA expression, respectively. Combinations of siRNAs targeting the same gene are also tested to determine if combinations of siRNAs enhance silencing.

Since Stat1 and Stat3 are used to activate the production of all the proinflammatory cytokines, whether Stat1 may be targeted to silence secretion of all 3 cytokines using one siRNA construct is also tested. Stat 1 and Stat3 are also important messengers for transducing the LPS signal, providing a further rationale for targeting them.

The effectiveness of suppressing inflammatory cytokine production when the inflammatory cytokines are targeted directly or indirectly are compared by ICC and ELISA analysis of culture supernatants (R&D Systems). An alternate approach is to silence expression of caspase-1, the protease used to process IL-1β and another proinflammatory cytokine, IL-18, into their active forms. Moreover, this makes sense since a caspase-1 inhibitor protects rats from LPS-induced death. A recent study suggests that IL-1β processing requires not only caspase-1 but a macromolecular complex (the inflammasome) (Martinon F, et al. (2002) Mol Cell, 10,417-426). When the details of how this complex functions are identified, other components, such as Pycard, may be targeted. Another approach is to construct delivery vectors capable of targeting more than one cytokine by using an internal ribosome entry site to produce more than one transcript or by constructing a single transcript with more than one complementary sequence separated by a loop.

Inhibition of the lethal consequences of proinflammatory cytokines without interfering with the important protective role of macrophages in initiating an immune response is important and therefore, it is determined whether blocking Stat1 or proinflammatory cytokine production interferes with the ability of macrophages to produce nondeleterious cytokines, such as IL-12 and IFNγ, and to process and present antigens to activate T lymphocytes. ICC staining is performed one day after LPS stimulation using antibodies to IFNγ and IL-12. To test antigen processing and presentation, siRNA-transfected MDMs are infected with recombinant vaccinia virus expressing HIV gag and incubated with thawed PBMCs from HIV-seropositive donors. The ability of silenced macrophages to activate a secondary T cell response is tested by measuring the enhancement of gag-specific cytotoxicity 7 days later, compared to that in untransfected macrophages or macrophages treated with irrelevant siRNA using routine methods.

### Example 4

### Silencing Fas Pathway Genes in Hepatocytes

In this example, silencing of fasL and more distal genes involved in fas-mediated apoptosis is compared with silencing of fas. Target sequences in human and murine fasL, FADD, caspase 8, bid and caspase 3 are identified and tested initially for protein and mRNA expression by Western blot, Northern blot and RNase protection assay (using the APO multiprobe mouse and human template sets from Pharmingen) in Jurkat cells, which express all of these genes, and in hepatocyte cell lines and purified mouse hepatocytes, which express all but fasL. Cells, which have been effectively silenced, are then compared to control cells for their ability to resist fas-mediated apoptosis using agonistic murine and human anti-fas antibodies and for their sensitivity to other forms of caspase-mediated apoptosis (i.e. etoposide, uv irradiation). Apoptotic assays measure annexin V staining, TUNEL staining and propidium iodide incorporation as well as caspase 3 and 8 activation using the fluorogenic detection Cytoxilux and Phiphilux-G kits (Oncolmmunin) (Liu L, et al. (2002) Nat Mod, 8, 185-189) by flow cytometry.

The therapeutic maneuver should not interfere with antigen-specific T cell-mediated cytolysis, which is mediated by release of cytolytic granules and is, in large part, caspase-independent. Therefore Cr release assay is used to test whether cells transfected with all of the silencing constructs are equally susceptible to lysis by PHA-activated T-cell lines (made from human PBMCs or mouse splenocytes, depending on the target species). To perform these assays, 4 ug/ml conA is added to the transfected target cells to make them universal T cell targets. Constructs which best silence fas-mediated killing without affecting other forms of apoptosis or T cell lysis are used to generate DNA and viral vectors as above. These are tested for their ability to establish a silenced state in vitro.

### Example 5

### Testing RNAi Strategies in Mouse Models of Fulminant Hepatitis

In this example, the most effective siRNAs from Example 4 are tested further in mouse models of fulminant hepatitis. The first fulminant hepatitis experiments are done in transgenic mice with fas silenced only in hepatocytes and with fasL silenced only in T lymphocytes. If the lethal consequences can be averted in the transgenic mice, then more translatable ways to delivery siRNA are tested. If the transgenic mice are not protected, transgenic mice are produced targeting other genes or combinations of target genes. Mice are initially challenged after transduction at the peak of silencing. Whether protection can be provided by treatment after the noxious insult is also tested.

It is believed that these targets will prevent death in the acute setting, but will not interfere with more general immune responses. To determine whether silencing of the chosen genes interfere with immune defense, it is also tested whether silenced mice are able to defend against two infectious agents (the gram positive bacterium Listeria monocytogenes and vaccinia virus). It is anticipated that immune protection against these pathogens will not be significantly compromised by silencing fas or proinflammatory cytokine production, provided an appropriate dose is chosen. The fas pathway does not have an important role in protective effector function, but is primarily used to tune-down and terminate the immune response to infection. Therefore it is not expected that silencing fas will have a significant effect on the protective immune response. Silencing the proinflammatory cytokines, however, may interfere with the generation of a protective response since transgenic mice expressing a soluble form of the TNF receptor are highly susceptible to some pathogens, including Listens monocytogenes.

The goal of the RNAi intervention is to shift the balance in the immune response by blocking as much as possible the harmful effects of inflammation without substantially turning down the protective responses. The best way to monitor whether this has been achieved is to test survival and toxicity after pathogenic challenge.

Experiments using hydrodynamic injection of duplex fas-siRNA to protect mice from conA and anti-fas antibody induced autoimmune hepatitis are expanded by investigating the protective effects of targeting other fas pathway genes and evaluating the pros and cons of other delivery methods. Transgenic mice with silenced fas in hepatocytes and silenced fasL in T lymphocytes are used. Then few of the most promising target siRNAs are chosen and one or two of the best delivery strategies identified for targeting hepatocytes are chosen. Lentiviral delivery of siRNA targeting fasL to lymphocytes and natural killer (NK) cells, the cells responsible for hepatic damage, is also an alternate approach to silencing the apoptotic pathway in hepatocytes. The analysis parallels that of the previous section with a shift in focus from macrophages to hepatocytes or lymphocytes.

It is also determined whether this therapeutic maneuver can provide protection when administered after challenge and whether siRNA targeting fas or apoptotic genes interferes with the protective response to Listeria or vaccinia virus. Post-exposure therapy may be able to be provided for the less fulminant hepatitis induced by conA and prevent the development of fibrosis/cirrhosis in this model. Since death is very rapid in the anti-fas antibody model (occurring within 6-8 hrs in most mice) protection of mice after exposure to anti-fas antibody is not expected. These experiments provide guidance for later primate studies development of RNAi for human use.

### Example 6

### Testing RNAi Strategies in Mouse Models of Sepsis

Silencing strategies for proinflammatory cytokines are tested in 2 mouse models of septic shock, LPS injection and intraperitoneal infection with the gram negative bacterium, E coli O111:B4 (Barton B E, and Jackson J V. (1993) Infect Immun, 61, 1496-1499). The mouse models used are based on the work of Lamping et al. ((1998) J. Clin Invest 101, 2065-2071, incorporated herein by reference), who inject C57BI/6 mice intraperitoneally with the sensitizing agent D-galactosamine (20 mg) plus low dose (2.5 ng) E coli O111:B4 LPS (Sigma). In this model elevated serum IL-1β, TNFα and IL-6 can be detected by ELISA 75 minutes later and largely resolve by 7 hours, at which time liver damage can be monitored by serum transaminase elevation. Within 12 hours, most of the mice die and the remaining approximately 35% of mice survive. Another model used is intraperitoneal injection with 10⁷ live E coli 0111:B4, which leads to death in approximately 90% of mice within a few days. One or a few siRNA delivery systems are chosen which effectively transduce macrophages in vivo. Several siRNA targets are also chosen which silence proinflammatory genes without affecting other macrophage functions. It is difficult to monitor the efficiency of in vivo silencing in healthy, unchallenged mice since these cytokines are only expressed after activation. Therefore the effect of RNAi will be tested first in BL/6 mice treated with sublethal doses of LPS or bacteria.

Mice are bled 1, 2 and 4 hours after sublethal challenge at which time proinflammatory cytokines will be detectable. If none are detected at these times in control mice, the dose is varied and if necessary mice are sacrificed to determine the optimal detection time by using ICC and RNase protection assays to follow cytokine protein and mRNA in peritoneal macrophages and in the liver. Cytokine expression is detected by immunofluorescence microscopy of whole mouse sections and by flow cytometry analysis of intracellular cytokine staining of tissue macrophages isolated from liver, spleen and lung. Northern blot and RPA is also performed on isolated macrophages. Mice treated with the different delivery vehicles and siRNA inserts are compared to mock treated mice and to mice treated with an irrelevant siRNA (i.e. GFP-siRNA).

For the lethal challenge experiments the optimal times to measure serum proinflammatory cytokines and liver enzymes (by ELISA, R&D Systems and clinical chemistry) are first determined, tissue proinflammatory cytokine mRNA and protein expression are determined, and death is determined. Mice are sacrificed for each measurement at the optimal detection time as well as 4-6 hours before the earliest death to count bacteria in the peritoneal cavity and serum. Histological analysis provides a qualitative assessment of vascular leak and intravascular coagulation. Vascular leak is quantitated by tail vein injection of fluorescent FITC-albumin if the qualitative analysis suggests that it would be informative. If silencing is confirmed in the sublethal challenge experiments, one day after transduction (or at the peak of silencing), targeted and control mice are challenged by intraperitoneal injection with lethal doses of LPS or bacteria.

Groups of 5 control and silenced mice are sacrificed at the previously determined times for monitoring proinflammatory cytokine protein and mRNA expression, serum cytokine levels and bacterial counts. Expression of other cytokines, such as IL-12 and IFNγ, are analyzed concurrently. An additional 10-20 mice are followed in each group until death. Terminal autopsies will look for signs of sepsis, organ failure and vascular leak. All surviving mice are sacrificed 10 days later and isolated liver and spleen macrophages are stimulated in vitro with IPS to determine whether proinflammatory cytokine production remains suppressed.

If protection is not observed or is incomplete, ways to enhance silencing, including increasing the numbers of treatments or dose, changing the route of injection, or using combinations of siRNAs targeting different proinflammatory cytokines are investigated. If protection is observed, the order of challenge and treatment with siRNAs is reversed to determine whether RNAi can protect after exposure and how long it is possible to extend the interval after exposure. This will require reducing the bacterial/LPS challenge so that the proportion of animals that die is lower and the time to death is increased. The same analysis of serum and intracellular cytokine expression is performed as previously described.

It is also examined whether suppression of proinflammatory cytokine production interferes with pathogen clearance and the development of an antigen-specific T cell response. siRNA treated and control C57BL/6 mice are injected i. p. with sublethal doses of Listeria monocytogenes (Lm-gp33, 5×10⁶ bacteria) or vaccinia virus (vv-gp33, 10⁵ pfu) expressing an LCMV gp33 T cell peptide recognized by T cells in BL/6 mice (Manjunath N, et al. (2001) J Clin Invest, 108, 871-878; Pircher H, et al. (1989) Nature , 342, 559-561). To test for the ability to control infection, groups of 5 mice are sacrificed six days after infection and analyzed by colony count or plaque forming assay for bacteria or virus in the liver or ovary, respectively. To test for immunocompetence, mouse splenocytes harvested from the same mice are stimulated in vitro with 1 ug/ml gp33 peptide and tested 7-10 days later for IFNγ production in response to gp33 peptide as described. The ability to contain these infections may be somewhat impaired if the amount of silencing is complete, since the proinflammatory cytokines contribute to the activation of macrophages for phagocytosis. However, since silencing is partial, there may be no detectable effect on clearance of these pathogens. If there is, it is determined whether a lower dose of siRNA can protect from death but still preserve antiviral and antibacterial defenses, both innate and adaptive.

Since genetically diverse animals may respond to infection and immune-based therapies differently, the experiments described in this section are repeated in another mouse strain (BALB/c) to begin to probe possible differences in siRNA effectiveness and toxicity. These two mouse strains (BALB/c and C57BL/6) differ in the ratios of T_{H}1 and T_{H}2 cytokines they produce in response to infection, and therefore handle infections differently. This has been best illustrated in a Leishmania infection model (Muller I, et al. (1989). Immunol Rev, 112, 95-113; Lohoff M, et al. (1989). Immunobiology, 179, 412-421). Because the gp33 peptide is not recognized in BALB/c mice, the immunodominant Listeria listeriolysin peptide GYKDGNEYI (residues 91-99) recognized by CD8 T cells in BALB/c mice in the experiments investigating T cell immunity is substituted (Villanueva M S, Sijts A J, and Pamer E G. (1995). J Immunol, 155, 5227-5233). For each mouse strain the therapeutic approach is independently optimized so that useful comparisons can be made.

### Example 7

### Determination of Persistence and Possible Long-Term Toxicity of RNA Interference In Vivo

In the in vivo study of RNAi targeting fas using duplex RNAi, above, it was found that silencing in hepatocytes was not diminished for at least 10 days in these mostly resting cells. Sustained silencing was also found for up to 3 weeks in in vitro transfected nondividing macrophages.

To determine how long duplex siRNAs can maintain the silenced state in vivo in the absence of endogenous expression from an integrated vector, transgenic-GFP (Tg-GFP) mice which globally express GFP are injected by tail vein with duplex GFP-siRNA, since the fluorescent readout is the simplest tool available. GFP-siRNA and irrelevant siRNA-treated mice are sacrificed 1, 10 and 25 days, and 2 months post injection and analyzed by fluorescence microscopy and in situ hybridization for GFP protein and mRNA expression in whole mouse sections and by flow cytometry, Northern blot, and quantitative RT-PCR for GFP expression in selected isolated cell populations. All assays are controlled by testing for expression of a non-silenced gene, such as β-actin. If silencing is stable for 2 months, it is tested at 6 months and, if positive, it can be assumed that silencing persists for the life of the mouse. The specific cells analyzed include hepatocytes and macrophages from the liver and other cells, including cells previously shown to be silenced. These studies illustrate not only whether the silenced state persists, but also whether it may have spread, for instance, from hepatocytes to tissue macrophages in the same tissue.

This analysis is repeated in mice treated with the methods for silencing proinflammatory cytokine and fas pathway genes that are shown above to be most attractive for developing for possible human use. If silencing persists, then prolonged protection is also tested by challenging mice at increasingly delayed times after siRNA treatment.

If the period of silencing is prolonged for more than 1-2 months, it is also determined whether silencing proinflammatory cytokines or the fas pathway has adverse consequences. Mice are treated when they are 6 wks of age and challenged with Listeria and vaccinia virus when they are old (approximately 2.5 yrs of age) and have begun to experience the immune functional decline that accompanies natural aging. Their ability to control infection is assessed as described by colony counts and plaque forming assays in the liver and ovaries, respectively, and by measuring the number of T cells activated to produce IFNγ in response to each of the pathogens. The age and cause of death is compared in treated, compared to wild-type mice, by terminal autopsy. The weight and histology of organs are compared. Because of the importance of the fas pathway in regulating the immune response, for fas gene-silenced mice, special attention is paid to the development of tumors and proliferative diseases, particularly of lymphocytes and other hematopoietic cells and to signs of autoimmunity in joints and kidneys. Groups of 10 fas-silenced and control mice are sacrificed at 2.5 years of age to compare blood, spleen and lymph node cell counts as well as liver histology.

Equivalents

Those skilled in the art will recognize, or be able to ascertain using no more than routine experimentation, many equivalents to the specific embodiments of the invention described herein. Such equivalents are intended to be encompassed by the following claims.

TABLE 1

| | Gene | |
|---|---|---|
| Description | Name | GenBank GI No. |
| Fas Pathway Molecules | | |
| CASP2 and RIPK1 domain containing adaptor with death domain; caspase and RIP | CRADD | 4503031 |
| adaptor with death domain; death adaptor molecule RAIDD; death domain containing | | |
| protein CRADD; RIP-associated ICH1/CED3-homologous protein with death domain; | | |
| RIP-associated protein with a death domain [Homo sapiens]. | | |
| Fas-associated via death domain; Fas-associating protein with death domain; Fas- | FADD | 4505229 |
| associating death domain-containing protein; mediator of receptor-induced toxicity | | |
| [Homo sapiens]. | | |
| Fas antigen; APO-1 cell surface antigen [Homo sapiens] | Fas (CD95 or | 23510434 |
| | APO-1) | |
| lymphotoxin alpha precursor; lymphotoxin A; tumor necrosis factor, beta; TNF | LTA | 6806893 |
| superfamily, member 1; tumor necrosis factor beta; lyphotoxin alpha (TNF | | |
| superfamily, member 1) [Homo sapiens]. | | |
| lymphotoxin-beta isoform b; lymphotoxin beta; tumor necrosis factor C [Homo sapiens]. | LTB | 6996016 |
| lymphotoxin-beta isoform a; lymphotoxin beta; tumor necrosis factor C [Homo sapiens]. | LTB | 4505035 |
| lymphotoxin beta receptor; lymphotoxin B receptor; tumor necrosis factor C receptor; | LTBR | 4505039 |
| tumor necrosis factor receptor superfamily, member 3; LT-beta-R [Homo sapiens]. | | |
| nuclear factor of kappa light polypeptide gene enhancer in B-cells 1 (p105); Nuclear | NFKB1 | 10835177 |
| factor of kappa light chain gene enhancer in B-cells 1 (p105); nuclear factor kappa-B, | | |
| subunit 1; NF-kappa B [Homo sapiens]. | | |
| nuclear factor of kappa light polypeptide gene enhancer in B-cells inhibitor, alpha; | NFKBIA | 10092619 |
| Nuclear factor of kappa light chain gene enhancer in B-cells [Homo sapiens]. | | |
| nuclear factor of kappa light polypeptide gene enhancer in B-cells inhibitor, beta | NFKBIB | 21361135 |
| [Homo sapiens]. | | |
| nuclear factor of kappa light polypeptide gene enhancer in B-cells inhibitor, epsilon | NFKBIE | 4758806 |
| [Homo sapiens]. | | |
| nuclear factor of kappa light polypeptide gene enhancer in B-cells inhibitor-like 1; | NFKBIL1 | 26787991 |
| inhibitor of kappa light chain gene enhancer in B cells-like; I-kappa-B-like protein; | | |
| IkBL protein [Homo sapiens]. | | |
| nerve growth factor receptor precursor [Homo sapiens]. | NGFR | 4505393 |
| Tumor necrosis factor (TNF)-alpha | TNFα | 25952111 |
| tumor necrosis factor receptor superfamily, member 5 isoform 2 precursor; CD40 | TNFRSF5 | 23312371 |
| antigen; CD40L receptor; CD40 type II isoform; nerve growth factor receptor-related B | | |
| lymphocyte activation molecule; B cell-associated molecule; B cell surface antigen | | |
| CD40 [Homo sapiens]. | | |
| tumor necrosis factor receptor superfamily, member 5 isoform 1 precursor; CD40 | TNFRSF5 | 4507581 |
| antigen; CD40L receptor; CD40 type II isoform; nerve growth factor receptor-related B | | |
| lymphocyte activation molecule; B cell-associated molecule; B cell surface antigen | | |
| CD40 [Homo sapiens]. | | |
| tumor necrosis factor receptor superfamily, member 6 isoform 1 precursor; apoptosis | TNFRSF6 | 4507583 |
| antigen 1; Fas antigen; APO-1 cell surface antigen [Homo sapiens]. | | |
| tumor necrosis factor receptor superfamily, member 6 isoform 2 precursor; apoptosis | TNFRSF6 | 23510421 |
| antigen 1; Fas antigen; APO-1 cell surface antigen [Homo sapiens]. | | |
| tumor necrosis factor receptor superfamily, member 6 isoform 3 precursor; apoptosis | TNFRSF6 | 23510423 |
| antigen 1; Fas antigen; APO-1 cell surface antigen [Homo sapiens]. | | |
| tumor necrosis factor receptor superfamily, member 6 isoform 4 precursor; apoptosis | TNFRSF6 | 23510425 |
| antigen 1; Fas antigen; APO-1 cell surface antigen [Homo sapiens]. | | |
| tumor necrosis factor receptor superfamily, member 6 isoform 4 precursor; apoptosis | TNFRSF6 | 23510427 |
| antigen 1; Fas antigen; APO-1 cell surface antigen [Homo sapiens]. | | |
| tumor necrosis factor receptor superfamily, member 6 isoform 5 precursor; apoptosis | TNFRSF6 | 23510429 |
| antigen 1; Fas antigen; APO-1 cell surface antigen [Homo sapiens]. | | |
| tumor necrosis factor receptor superfamily, member 6 isoform 6 precursor; apoptosis | TNFRSF6 | 23510431 |
| antigen 1; Fas antigen; APO-1 cell surface antigen [Homo sapiens]. | | |
| tumor necrosis factor receptor superfamily, member 7 precursor; CD27 antigen; T cell | TNFRSF7 | 4507587 |
| activation antigen CD27; T cell antivation antigen S152; CD27L receptor [Homo sapiens]. | | |
| tumor necrosis factor receptor superfamily, member 8 isoform 2; Ki-1 antigen; CD30 | TNFRSF8 | 23510437 |
| antigen; cytokine receptor CD30; lymphocyte activation antigen CD30; CD30L | | |
| receptor [Homo sapiens]. | | |
| tumor necrosis factor receptor superfamily, member 8 isoform 1 precursor; Ki-1 | TNFRSF8 | 4507589 |
| antigen; CD30 antigen; cytokine receptor CD30; lymphocyte activation antigen CD30; | | |
| CD30L receptor [Homo sapiens]. | | |
| tumor necrosis factor receptor superfamily, member 10a; death receptor 4; cytotoxic | TNFRSF10A | 21361086 |
| TRAIL receptor; TNF-related apoptosis inducing ligand receptor 1 [Homo sapiens]. | | |
| tumor necrosis factor receptor superfamily, member 10b isoform 1 precursor; death | TNFRSF10B | 22547116 |
| receptor 5; TNF-related apoptosis-inducing ligand receptor 2; TRAIL receptor 2; Fas- | | |
| like protein precursor; apoptosis inducing receptor TRAIL-R2; apoptosis inducing | | |
| protein TRICK2A/2B; tumor necrosis factor receptor-like protein ZTNFR9; death | | |
| domain containing receptor for TRAIL/Apo-2L; cytotoxic TRAIL receptor-2; p53- | | |
| regulated DNA damage-inducible cell death receptor(killer) [Homo sapiens]. | | |
| tumor necrosis factor receptor superfamily, member 10b isoform 2 precursor; death | TNFRSF10B | 22547119 |
| receptor 5; TNF-related apoptosis-inducing ligand receptor 2; TRAIL receptor 2; Fas- | | |
| like protein precursor; apoptosis inducing receptor TRAIL-R2; apoptosis inducing | | |
| protein TRICK2A/2B; tumor necrosis factor receptor-like protein ZTNFR9; death | | |
| domain containing receptor for TRAIL/Apo-2L; cytotoxic TRAIL receptor-2; p53- | | |
| regulated DNA damage-inducible cell death receptor(killer) [Homo sapiens]. | | |
| tumor necrosis factor receptor superfamily, member 10c precursor; decoy receptor 1; | TNFRSF10C | 22547121 |
| TRAIL receptor 3; antagonist decoy receptor for TRAIL/Apo-2L; TNF related TRAIL | | |
| receptor; lymphocyte inhibitor of TRAIL; decoy without an intracellular domain; TNF | | |
| related apoptosis-inducing ligand receptor 3 [Homo sapiens]. | | |
| tumor necrosis factor receptor superfamily, member 10d precursor; decoy receptor 2; | TNFRSF10D | 22547108 |
| TNF receptor-related receptor for TRAIL; decoy with truncated death domain; TRAIL | | |
| receptor 4; TRAIL receptor with a truncated death domain [Homo sapiens]. | | |
| osteoprotegerin precursor; tumor necrosis factor receptor superfamily, member 11b; | TNFRSF11B | 22547123 |
| osteoprotegerin; osteoclastogenesis inhibitory factor [Homo sapiens]. | | |
| tumor necrosis factor receptor superfamily, member 25 isoform 11 precursor; | TNFRSF25 | 23200039 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 10 precursor; | TNFRSF25 | 23200037 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 9 precursor; | TNFRSF25 | 23200035 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 8 precursor; | TNFRSF25 | 23200033 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 7 precursor; | TNFRSF25 | 23200031 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 6 precursor; | TNFRSF25 | 23200029 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 5 precursor; | TNFRSF25 | 23200027 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 4 precursor; | TNFRSF25 | 23200025 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 3 precursor; | TNFRSF25 | 23200023 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 1 precursor; | TNFRSF25 | 23200021 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| tumor necrosis factor receptor superfamily, member 25 isoform 2 precursor; | TNFRSF25 | 4507569 |
| translocating chain-association membrane protein; death domain receptor 3; death | | |
| domain receptor 3 soluble form; apoptosis-mediating receptor; lymphocyte associated | | |
| receptor of death; death receptor beta; apoptosis inducing receptor; WSL-1 protein; | | |
| tumor necrosis factor receptor superfamily, member 12 [Homo sapiens]. | | |
| TNFRSF1A-associated via death domain isoform 1; tumor necrosis factor receptor-1- | TRADD | 13378137 |
| associated protein; TNFR1-associated death domain protein; tumor necrosis factor | | |
| receptor type 1 associated death domain protein [Homo sapiens]. | | |
| TNFRSF1A-associated via death domain isoform 2; tumor necrosis factor receptor-1- | TRADD | 24234726 |
| associated protein; TNFR1-associated death domain protein; tumor necrosis factor | | |
| receptor type 1 associated death domain protein [Homo sapiens]. | | |
| Tumor necrosis factor-associated apoptosis-inducing ligand (TRAIL) death-receptor | TRAIL | 23510439 |
| | (TNFSF10, | |
| | TL2, APO2L, | |
| | or Apo-2L) | |
| Caspase Family Molecules | | |
| Group I Caspases | | |
| Caspase 1 isoform alpha precursor; interleukin 1-beta convertase; interleukin 1-B | CASP1 | 15431328 |
| converting enzyme; IL1B-convertase [Homo sapiens]. | | |
| Caspase 1 isoform beta precursor; interleukin 1-beta convertase; interleukin 1-B | CASP1 | 4502573 |
| converting enzyme; IL1B-convertase [Homo sapiens]. | | |
| Caspase 1 isoform delta; interleukin 1-beta convertase; interleukin 1-B converting | CASP1 | 15431332 |
| enzyme; IL1B-convertase [Homo sapiens]. | | |
| Caspase 1 isoform epsilon; interleukin 1-beta convertase; interleukin 1-B converting | CASP1 | 15431334 |
| enzyme; IL1B-convertase [Homo sapiens]. | | |
| Caspase 1 isoform gamma precursor; interleukin 1-beta convertase; interleukin 1-B | CASP1 | 15431330 |
| converting enzyme; IL1B-convertase [Homo sapiens]. | | |
| Caspase 4 isoform alpha precursor; apoptotic cysteine protease Mih1/TX [Homo sapiens]. | CASP4 | 4502577 |
| Caspase 4 isoform delta precursor; apoptotic cysteine protease Mih1/TX [Homo sapiens]. | CASP4 | 15451912 |
| Caspase 4 isoform gamma precursor; apoptotic cysteine protease Mih1/TX [Homo sapiens]. | CASP4 | 15451910 |
| Caspase 5, precursor; TY protease [Homo sapiens]. | CASP5 | 4757914 |
| Group II Caspases | | |
| Caspase 2 isoform 2 precursor; ICH-1 protease; NEDD2 apoptosis regulatory gene | CASP2 | 4502575 |
| [Homo sapiens]. | | |
| caspase 2 isoform 3; ICH-1 protease; NEDD2 apoptosis regulatory gene [Homo sapiens]. | CASP2 | 14790182 |
| caspase 2 isoform 4; ICH-1 protease; NEDD2 apoptosis regulatory gene [Homo sapiens]. | CASP2 | 14790186 |
| Caspase 3 preproprotein; Yama; apopain; PARP cleavage protease; cysteine protease | CASP3 | 14790115 |
| CPP32; SREBP cleavage activity 1 [Homo sapiens]. | | |
| Caspase 3 preproprotein; Yama; apopain; PARP cleavage protease; cysteine protease | CASP3 | 14790119 |
| CPP32; SREBP cleavage activity 1 [Homo sapiens]. | | |
| Caspase 7 isoform beta; ICE-like apoptotic protease 3; apoptotic protease MCH-3; | CASP7 | 15718702 |
| Lice2 alpha/beta/gamma [Homo sapiens]. | | |
| Caspase 7 isoform alpha precursor; ICE-like apoptotic protease 3; apoptotic protease | CASP7 | 4502581 |
| MCH-3; Lice2 alpha/beta/gamma [Homo sapiens]. | | |
| Caspase 7 isoform alpha, large subunit; ICE-like apoptotic protease 3; apoptotic | CASP7 | 5718700 |
| protease MCH-3; Lice2 alpha/beta/gamma [Homo sapiens]. | | |
| Group III Caspases | | |
| Caspase 6 isoform alpha preproprotein; apoptotic protease MCH-2 [Homo sapiens]. | CASP6 | 14916483 |
| caspase 6 isoform beta; apoptotic protease MCH-2 [Homo sapiens]. | CASP6 | 14916485 |
| caspase 8 isoform A; Mch5 isoform alpha; FADD-homologous ICE/CED-3-like | CASP8 | 15718704 |
| protease; MACH-alpha-1/2/3 protein; MACH-beta-1/2/3/4 protein [Homo sapiens]. | | |
| caspase 8 isoform B; Mch5 isoform alpha; FADD-homologous ICE/CED-3-like | CASP8 | 15718706 |
| protease; MACH-alpha-1/2/3 protein; MACH-beta-1/2/3/4 protein [Homo sapiens]. | | |
| caspase 8 isoform C; Mch5 isoform alpha; FADD-homologous ICE/CED-3-like | CASP8 | 15718708 |
| protease; MACH-alpha-1/2/3 protein; MACH-beta-1/2/3/4 protein [Homo sapiens]. | | |
| caspase 8 isoform D; Mch5 isoform alpha; FADD-homologous ICE/CED-3-like | CASP8 | 15718710 |
| protease; MACH-alpha-1/2/3 protein; MACH-beta-1/2/3/4 protein [Homo sapiens]. | | |
| caspase 8 isoform E; Mch5 isoform alpha; FADD-homologous ICE/CED-3-like | CASP8 | 15718712 |
| protease; MACH-alpha-1/2/3 protein; MACH-beta-1/2/3/4 protein [Homo sapiens]. | | |
| Caspase-8 precursor (ICE-like apoptotic protease 5) (MORT1-associated CED-3 | CASP8 | 2493531 |
| homolog) (MACH) (FADD-homologous ICE/CED-3-like protease) (FADD-like ICE) | CASP8 | 2493531 |
| (FLICE) (Apoptotic cysteine protease) (Apoptotic protease Mch-5) (CAP4). | | |
| Caspase 9 isoform alpha preproprotein; ICE-like apoptotic protease 6; apoptotic | CASP9 | 14790124 |
| protease MCH-6; apoptotic protease activating factor 3 [Homo sapiens]. | | |
| Caspase 9 isoform beta preproprotein; ICE-like apoptotic protease 6; apoptotic | CASP9 | 14790128 |
| protease MCH-6; apoptotic protease activating factor 3 [Homo sapiens]. | | |
| Caspase-9 precursor (CASP-9) (ICE-like apoptotic protease 6) (ICE-LAP6) (Apoptotic | CASP9 | 28558771 |
| protease Mch-6) (Apoptotic protease activating factor 3) (APAF-3). | | |
| Caspase-9S precursor [Homo sapiens]. | caspase-9 varian | 4240552 |
| Caspase 10 isoform a preproprotein; FADD-like ICE2; apoptotic protease MCH-4; ICE | CASP10 | 4502569 |
| like apoptotic protease 4; Fas-associated death domain protein; interleukin-1B- | | |
| converting enzyme 2 [Homo sapiens]. | | |
| Caspase 10 isoform b preproprotein; FADD-like ICE2; apoptotic protease MCH-4; | CASP10 | 14916488 |
| ICE-like apoptotic protease 4; Fas-associated death domain protein; interleukin-1B- | | |
| converting enzyme 2 [Homo sapiens]. | | |
| Caspase 10 isoform c; FADD-like ICE2; apoptotic protease MCH-4; ICE-like | CASP10 | 14916490 |
| apoptotic protease 4; Fas-associated death domain protein; interleukin-1B-converting | | |
| enzyme 2 [Homo sapiens]. | | |
| Caspase 10 isoform d preproprotein; FADD-like ICE2; apoptotic protease MCH-4; | CASP10 | 14916492 |
| ICE-like apoptotic protease 4; Fas-associated death domain protein; interleukin-1B- | | |
| converting enzyme 2 [Homo sapiens]. | | |
| Homo sapiens CASP8 and FADD-like apoptosis regulator (CFLAR) peroxisome | FLIP (CASH, | 21361768 |
| proliferator activated receptor alpha (PPAR alpha) | MRIT, | |
| | CLARP, | |
| | FLAME, | |
| | Casper, | |
| | FLAME-1, I- | |
| | FLICE, | |
| | USURPIN) | |
| Other Caspases | | |
| Caspase 14 precursor; apoptosis-related cysteine protease [Homo sapiens]. | CASP14 | 6912286 |
| Mitochondrial Pathway Molecules | | |
| BCL2-antagonist of cell death protein; BCL2-binding protein; BCL2-binding | BAD | 10835069 |
| component 6; BCL-X/BCL-2 binding protein [Homo sapiens]. | | |
| BCL2-antagonist of cell death protein; BCL2-binding protein; BCL2-binding | BAD | 14670388 |
| component 6; BCL-X/BCL-2 binding protein [Homo sapiens]. | | |
| BCL2-associated athanogene; BCL2-associated athanogene 1 [Homo sapiens]. | BAG1 | 7949008 |
| BCL2-associated athanogene 2; BAG-family molecular chaperone regulator-2 [Homo sapiens]. | BAG2 | 4757834 |
| BCL2-associated athanogene 3; BAG-family molecular chaperone regulator-3; | BAG3 | 14043024 |
| docking protein CAIR-1; Bcl-2-binding protein; BCL2-binding athanogene 3 [Homo sapiens]. | | |
| BCL2-associated athanogene 4; silencer of death domains; BAG-family molecular | BAG4 | 6631075 |
| chaperone regulator-4 [Homo sapiens]. | | |
| BCL2-associated athanogene 5; BAG-family molecular chaperone regulator-5 [Homo sapiens]. | BAG5 | 6631077 |
| BCL2-antagonist/killer 1; cell death inhibitor 1 [Homo sapiens]. | Bak1 | 595926 |
| BCL2-associated X protein isoform alpha; apoptosis regulator BAX [Homo sapiens]. | BAX | 20631958 |
| BCL2-associated X protein isoform beta; apoptosis regulator BAX [Homo sapiens]. | BAX | 4757838 |
| BCL2-associated X protein isoform delta; apoptosis regulator BAX [Homo sapiens]. | BAX | 20631964 |
| BCL2-associated X protein isoform epsilon; apoptosis regulator BAX [Homo sapiens]. | BAX | 20631967 |
| BCL2-associated X protein isoform gamma; apoptosis regulator BAX [Homo sapiens]. | BAX | 20631961 |
| BCL2-associated X protein isoform sigma; apoptosis regulator BAX [Homo sapiens]. | BAX | 20631973 |
| BCL2 binding component 3; Bcl-2 binding component 3; PUMA/JFY1 protein; Bcl-2 | BBC3 | 15193488 |
| binding component 3 [Homo sapiens]. | | |
| B-cell lymphoma protein 2 alpha [Homo sapiens]. | BCL2 | 4557355 |
| B-cell lymphoma protein 2 beta [Homo sapiens]. | BCL2 | 4557357 |
| BCL2-related protein A1; hematopoietic BCL2-related protein A1 [Homo sapiens]. | BCL2A1 | 4757840 |
| BCL2-like 1 isoform 1 [Homo sapiens]. | BCL2L1; Bcl-xl | 20336335 |
| BCL2-like 11 isoform 1; bcl-2 interacting protein Bim; bcl-2 interacting mediator of | BCL2L11 | 20336315 |
| cell death; bcl-2-related ovarian death agonist [Homo sapiens]. | | |
| BCL2-like 11 isoform 2; bcl-2 interacting protein Bim; bcl-2 interacting mediator of | BCL2L11 | 20336317 |
| cell death; bcl-2-related ovarian death agonist [Homo sapiens]. | | |
| BCL2-like 11 isoform 3; bcl-2 interacting protein Bim; bcl-2 interacting mediator of | BCL2L11 | 20336319 |
| cell death; bcl-2-related ovarian death agonist [Homo sapiens]. | | |
| BCL2-like 11 isoform 4; bcl-2 interacting protein Bim; bcl-2 interacting mediator of | BCL2L11 | 20336321 |
| cell death; bcl-2-related ovarian death agonist [Homo sapiens]. | | |
| BCL2-like 11 isoform 5; bcl-2 interacting protein Bim; bcl-2 interacting mediator of | BCL2L11 | 20336323 |
| cell death; bcl-2-related ovarian death agonist [Homo sapiens]. | | |
| BCL2-like 11 isoform 6; bcl-2 interacting protein Bim; bcl-2 interacting mediator of | BCL2L11 | 5729740 |
| cell death; bcl-2-related ovarian death agonist [Homo sapiens]. | | |
| BCL2-like 11 isoform 7; bcl-2 interacting protein Bim; bcl-2 interacting mediator of | BCL2L11 | 20336325 |
| cell death; bcl-2-related ovarian death agonist [Homo sapiens]. | | |
| BCL2-like 11 isoform 8; bcl-2 interacting protein Bim; bcl-2 interacting mediator of | BCL2L11 | 20336327 |
| cell death; bcl-2-related ovarian death agonist [Homo sapiens]. | | |
| BCL2-like 2 protein; apoptosis regulator BCL-W [Homo sapiens]. | BCL2L2 | 4757842 |
| BCL2-like 13 (apoptosis facilitator); MIL1 protein [Homo sapiens]. | BCL2L13 | 7662506 |
| B-cell lymphoma 6 protein; B-cell CLL/lymphoma-6; cys-his2 zinc finger transcription | Bcl-6 | 21040336 |
| factor; zinc finger protein 51; lymphoma-associated zinc finger gene on chromosome 3 | | |
| [Homo sapiens]. | | |
| BH3 interacting domain death agonist; BH3-interacting domain death agonist [Homo sapiens]. | BID | 4557361 |
| BCL2-interacting killer, apoptosis-inducing NBK [Homo sapiens]. | BIK | 4502411 |
| baculoviral IAP repeat-containing protein 2; apoptosis inhibitor 1; cIAP1; hiap-2; | BIRC2 | 4502141 |
| NFR2-TRAF signalling complex protein [Homo sapiens]. | | |
| baculoviral IAP repeat-containing protein 3; apoptosis inhibitor 2; TNFR2-TRAF | BIRC3 | 4502139 |
| signalling complex protein [Homo sapiens]. | | |
| baculoviral IAP repeat-containing protein 4; X-linked inhibitor of apoptosis; apoptosis | BIRC4 | 32528299 |
| inhibitor 3 [Homo sapiens]. | | |
| baculoviral IAP repeat-containing protein 5; apoptosis inhibitor 4; survivin [Homo sapiens]. | BIRC5 | 4502145 |
| Bcl-2 modifying factor [Homo sapiens]. | BMF | 15723378 |
| BCL2/adenovirus E1B 19 kD interacting protein 1 isoform BNIP1; BCL2/adenovirus | BNIP1 | 4502441 |
| E1B 19 kD-interacting protein 1; Nip1; BCL2/adenovirus E1B 19 kD interacting protein | | |
| 1 [Homo sapiens]. | | |
| BCL2/adenovirus E1B 19 kD interacting protein 1 isoform BNIP1-a; BCL2/adenovirus | BNIP1 | 7524348 |
| E1B 19 kD-interacting protein 1; Nip1; BCL2/adenovirus E1B 19 kD interacting protein | | |
| 1 [Homo sapiens]. | | |
| BCL2/adenovirus E1B 19 kD interacting protein 1 isoform BNIP1-b; BCL2/adenovirus | BNIP1 | 7524350 |
| E1B 19 kD-interacting protein 1; Nip1; BCL2/adenovirus E1B 19 kD interacting protein | | |
| 1 [Homo sapiens]. | | |
| BCL2/adenovirus E1B 19 kD interacting protein 1 isoform BNIP1-c; BCL2/adenovirus | BNIP1 | 7524352 |
| E1B 19 kD-interacting protein 1; Nip1; BCL2/adenovirus E1B 19 kD interacting protein | | |
| 1 [Homo sapiens]. | | |
| BCL2/adenovirus E1B 19 kD interacting protein 2; BCL2/adenovirus E1B 19 kD- | BNIP2 | 4757856 |
| interacting protein 2; Nip2 [Homo sapiens]. | | |
| BCL2/adenovirus E1B 19 kD interacting protein 3; BCL2/adenovirus E1B 19 kD- | BNIP3 | 7669481 |
| interacting protein 3; Nip3 [Homo sapiens]. | | |
| BCL2/adenovirus E1B 19 kD-interacting protein 3-like; Nix; BCL2/adenovirus E1B 19- | BNIP3L | 4757860 |
| kd protein-interacting protein 3a; BCL2/adenovirus E1B 19 kD interacting protein 3- | | |
| like; adenovirus E1B19 k-binding protein B5 [Homo sapiens]. | | |
| BCL2-related ovarian killer [Homo sapiens]. | BOK | 14210524 |
| harakiri; activator of apoptosis Hrk; harakiri, BCL2-interacting protein (contains only | HRK | 4504493 |
| BH3 domain); BCL2-interacting protein [Homo sapiens]. | | |
| myeloid cell leukemia sequence 1 isoform 2; induced myeloid leukemia cell | MCL1 | 33519458 |
| differentiation protein Mcl-1 [Homo sapiens]. | | |
| myeloid cell leukemia sequence 1 isoform 1; induced myeloid leukemia cell | MCL1 | 11386165 |
| differentiation protein Mcl-1 [Homo sapiens]. | | |
| non-metastatic cells 2, protein (NM23B) expressed in; Non-metastatic cells 2, protein | NME2 | 4505409 |
| (NM23) expressed in [Homo sapiens]. | | |
| phorbol-12-myristate-13-acetate-induced protein 1; adult T cell leukemia-derived PMA | PMAIP1 | 10863923 |
| responsive [Homo sapiens]. | | |
| ProInflammatory Cytokine Molecules | | |
| colony stimulating factor 2 precursor, sargramostim; granulocyte-macrophage colony | CSF2 | 27437030 |
| stimulating factor; molgramostin [Homo sapiens]. | | |
| colony stimulating factor 3 isoform a precursor; granulocyte colony stimulating factor; | CSF3 | 4503079 |
| pluripoietin; filgrastim; lenograstim [Homo sapiens]. | | |
| colony stimulating factor 3 isoform c; granulocyte colony stimulating factor; | CSF3 | 27437051 |
| pluripoietin; filgrastim; lenograstim [Homo sapiens]. | | |
| colony stimulating factor 3 isoform b; granulocyte colony stimulating factor; | CSF3 | 27437049 |
| pluripoietin; filgrastim; lenograstim [Homo sapiens]. | | |
| colony stimulating factor 3 receptor isoform d precursor; granulocyte colony | CSF3 | 27437045 |
| stimulating factor receptor; CD114 antigen [Homo sapiens]. | | |
| colony stimulating factor 3 receptor isoform b precursor; granulocyte colony | CSF3R | 27437042 |
| stimulating factor receptor; CD114 antigen [Homo sapiens]. | | |
| colony stimulating factor 3 receptor isoform c precursor; granulocyte colony | CSF3R | 24496783 |
| stimulating factor receptor; CD114 antigen [Homo sapiens]. | | |
| colony stimulating factor 3 receptor isoform a precursor; granulocyte colony | CSF3R | 4503081 |
| stimulating factor receptor; CD114 antigen [Homo sapiens]. | | |
| interleukin 1, alpha proprotein; preinterleukin 1 alpha; hematopoietin-1 [Homo sapiens]. | IL1A | 27894330 |
| interleukin 1, beta proprotein; preinterleukin 1 beta; interleukin 1; catabolin [Homo sapiens]. | IL1B | 10835145 |
| interleukin 1 receptor, type I precursor; interleukin 1 receptor alpha, type I; interleukin | IL1R1 | 4504659 |
| receptor 1; antigen CD121a [Homo sapiens]. | | |
| interleukin 1 receptor, type II precursor; type II interleukin-1 receptor, beta; antigen | IL1R2 | 4758598 |
| CDw121b [Homo sapiens]. | | |
| interleukin 1 receptor, type II precursor; type II interleukin-1 receptor, beta; antigen | IL1R2 | 27894334 |
| CDw121b [Homo sapiens]. | | |
| interleukin 2 precursor; T cell growth factor; aldesleukin [Homo sapiens]. | IL2 | 28178861 |
| interleukin 2 receptor, alpha chain precursor [Homo sapiens]. | IL2RA | 4557667 |
| interleukin 2 receptor beta precursor; CD122 antigen; high affinity IL-2 receptor beta | IL2RB | 4504665 |
| subunit [Homo sapiens]. | | |
| interleukin 2 receptor, gamma chain, precursor; Interleukin-2 receptor, gamma; | IL2RG | 4557882 |
| common cytokine receptor gamma chain; CD132 [Homo sapiens]. | | |
| interleukin 3 precursor; multilineage-colony-stimulating factor; hematopoietic growth | IL3 | 28416915 |
| factor; P-cell stimulating factor; mast-cell growth factor [Homo sapiens]. | | |
| interleukin 3 receptor, alpha precursor; Interleukin-3 receptor; CD123 antigen [Homo sapiens]. | IL3RA | 13324710 |
| interleukin 4 isoform 1 precursor; B_cell stimulatory factor 1; lymphocyte stimulatory | IL4 | 4504669 |
| factor 1 [Homo sapiens]. | | |
| interleukin 4 isoform 2 precursor; B_cell stimulatory factor 1; lymphocyte stimulatory | IL4 | 27477092 |
| factor 1 [Homo sapiens]. | | |
| interleukin 4 receptor precursor; CD124 [Homo sapiens]. | IL4R | 4557669 |
| interleukin 5 precursor; interleukin-5; T-cell replacing factor; B cell differentiation | IL5 | 4504671 |
| factor I; eosinophil differentiation factor [Homo sapiens]. | | |
| interleukin 5 receptor, alpha isoform 1 precursor; interleukin 5 receptor type 3; | IL5RA | 28559027 |
| interleukin-5 receptor; interleukin-5 receptor alpha subunit; interleukin-5 receptor | | |
| alpha chain; CD125 antigen [Homo sapiens]. | | |
| interleukin 5 receptor, alpha isoform 1 precursor; interleukin 5 receptor type 3; | IL5RA | 28559021 |
| interleukin-5 receptor; interleukin-5 receptor alpha subunit; interleukin-5 receptor | | |
| alpha chain; CD125 antigen [Homo sapiens]. | | |
| interleukin 5 receptor, alpha isoform 2 precursor; interleukin 5 receptor type 3; | IL5RA | 28559023 |
| interleukin-5 receptor; interleukin-5 receptor alpha subunit; interleukin-5 receptor | | |
| alpha chain; CD125 antigen [Homo sapiens]. | | |
| interleukin 5 receptor, alpha isoform 2 precursor; interleukin 5 receptor type 3; | IL5RA | 28559029 |
| interleukin-5 receptor; interleukin-5 receptor alpha subunit; interleukin-5 receptor | | |
| alpha chain; CD125 antigen [Homo sapiens]. | | |
| interleukin 5 receptor, alpha isoform 3 precursor; interleukin 5 receptor type 3; | IL5RA | 28559025 |
| interleukin-5 receptor; interleukin-5 receptor alpha subunit; interleukin-5 receptor | | |
| alpha chain; CD125 antigen [Homo sapiens]. | | |
| interleukin 5 receptor, alpha isoform 3 precursor; interleukin 5receptor type 3; | IL5RA | 28559031 |
| interleukin-5 receptor; interleukin-5 receptoralpha subunit; interleukin-5 receptor alpha | | |
| chain; CD125 antigen [Homo sapiens]. | | |
| interleukin 7 precursor [Homo sapiens]. | IL7 | 4504677 |
| interleukin 7 receptor precursor; CD127 antigen; interleukin 7 receptor alpha chain | IL7R | 28610151 |
| [Homo sapiens] | | |
| interleukin 8 precursor; LUCT/interleukin-8; monocyte derived neutrophil-activating | IL8 | 10834978 |
| protein; monocyte-derived neutrophil chemotactic factor; neutrophil-activating factor; | | |
| neutrophil-activating peptide 1; neutrophil-activating protein 1; lymphocyte-derived | | |
| neutrophil-activating factor; T cell chemotactic factor; granulocyte chemotactic protein | | |
| 1; CXC chemokine ligand 8; beta-thromboglobulin-like protein; protein 3-10C; | | |
| emoctakin; small inducible cytokine subfamily B, member 8 [Homo sapiens]. | | |
| interleukin 8 receptor alpha; chemokine (C—X—C) receptor 1; high affinity interleukin-8 | IL8RA | 4504681 |
| receptor A; IL-8 receptor type 1; interleukin-8 receptor type 1; interleukin-8 receptor | | |
| type A; IL-8 receptor [Homo sapiens]. | | |
| interleukin 8 receptor beta; chemokine (CXC) receptor 2; high affinity interleukin-8 | IL8RB | 4504683 |
| receptor B; GRO/MGSA receptor; interleukin 8 receptor B; interleukin 8 receptor; | | |
| CXCR2 gene for IL8 receptor type B; interleukin 8 receptor type 2 [Homo sapiens]. | | |
| interleukin 9 precursor; p40 T-cell and mast cell growth factor; T-cell growth factor | IL9 | 10834980 |
| p40; p40 cytokine; p40 protein; homolog of mouse T cell and mast cell growth factor | | |
| 40 [Homo sapiens]. | | |
| interleukin 9 receptor isoform 1 precursor [Homo sapiens]. | IL9R | 29171682 |
| interleukin 9 receptor isoform 2 [Homo sapiens] | IL9R | 29171684 |
| interleukin 10 precursor; cytokine synthesis inhibitory factor [Homo sapiens]. | IL10 | 10835141 |
| interleukin 10 receptor, alpha precursor [Homo sapiens]. | IL10RA | 4504633 |
| interleukin 10 receptor, beta precursor; cytokine receptor family II, member 4; cytokine | IL10RB | 24430215 |
| receptor class-II CRF2-4 [Homo sapiens]. | | |
| interleukin 11 precursor; oprelvekin; adipogenesis inhibitory factor [Homo sapiens]. | IL11 | 10834994 |
| interleukin 11 receptor, alpha isoform 1 precursor; interleukin-11 receptor alpha chain | IL11RA | 4758594 |
| [Homo sapiens]. | | |
| interleukin 11 receptor, alpha isoform 2 precursor; interleukin-11 receptor alpha chain | IL11RA | 22212922 |
| [Homo sapiens]. | | |
| interleukin 12A precursor; interleukin 12A; natural killer cell stimulatory factor 1, 35 | IL12A | 24430219 |
| kD subunit; cytotoxic lymphocyte maturation factor 1, p35; interleukin 12, p35; IL-12, | | |
| subunit p35; NF cell stimulatory factor chain 1; interleukin-12 alpha chain precursor | | |
| [Homo sapiens]. | | |
| interleukin 12B precursor; natural killer cell stimulatory factor-2; interleukin 12B; | IL12B | 24497438 |
| cytotoxic lymphocyte maturation factor 2, p40; interleukin-12 beta chain; interleukin | | |
| 12, p40; natural killer cell stimulatory factor, 40 kD subunit; IL12, subunit p40 [Homo sapiens]. | | |
| interleukin 12 receptor, beta 1 isoform 1 precursor; IL-12 receptor beta component; | IL12RB1 | 5031785 |
| interleukin-12 receptor beta-1 chain [Homo sapiens]. | | |
| interleukin 12 receptor, beta-1 isoform 2 precursor; IL-12 receptor beta component; | IL12RB1 | 24497440 |
| interleukin-12 receptor beta-1 chain [Homo sapiens]. | | |
| interleukin 12 receptor, beta 2 precursor; IL-12 receptor beta 2; interleukin-12 receptor | IL12RB2 | 4504643 |
| beta-2 chain [Homo sapiens]. | | |
| interleukin 13 precursor [Homo sapiens]. | IL13 | 26787978 |
| interleukin 13 receptor, alpha 1 precursor; IL13 receptor alpha-1 chain; CD213a1 | IL13RA1 | 4504647 |
| antigen [Homo sapiens]. | | |
| interleukin 13 receptor, alpha 2 precursor; interleukin 13 binding protein; interleukin | IL13RA2 | 10834992 |
| 13 receptor alpha 2 chain; IL-13 receptor [Homo sapiens]. | | |
| interleukin 15 isoform 2 precursor [Homo sapiens]. | IL15 | 26787986 |
| interleukin 15 isoform 1 precursor [Homo sapiens]. | IL15 | 10835153 |
| interleukin 15 isoform 1 precursor [Homo sapiens]. | IL15 | 26787984 |
| interleukin 15 receptor, alpha isoform 1 precursor [Homo sapiens]. | IL15RA | 4504649 |
| interleukin 15 receptor, alpha isoform 2 [Homo sapiens]. | IL15RA | 26787982 |
| interleukin 16 isoform 1 proprotein [Homo sapiens]. | IL16 | 27262655 |
| interleukin 16 isoform 2; lymphocyte chemoattractant factor [Homo sapiens]. | IL16 | 27262657 |
| interleukin 17 precursor; cytotoxic T-lymphocyte-associated serine esterase 8; | IL17 | 4504651 |
| cytotoxic T-lymphocyte-associated antigen 8 [Homo sapiens]. | | |
| interleukin 17B precursor; interleukin 20; cytokine-like protein ZCYTO7; neuronal | IL17B | 7657228 |
| interleukin-17 related factor; interleukin-17 beta [Homo sapiens]. | | |
| interleukin 17C; cytokine CX2 [Homo sapiens]. | IL17C | 7019435 |
| interleukin 17D precursor; interleukin 27 [Homo sapiens]. | IL17D | 19923715 |
| interleukin 17E isoform 1 precursor; interleukin 25 [Homo sapiens]. | IL17E | 12232485 |
| interleukin 17E isoform 2; interleukin 25 [Homo sapiens]. | IL17E | 27477081 |
| interleukin 17F isoform 1 precursor; interleukin-24; cytokine ML-1 [Homo sapiens]. | IL17F | 16418375 |
| interleukin 17F isoform 2; interleukin-24; cytokine ML-1 [Homo sapiens]. | IL17F | 27477084 |
| interleukin 17 receptor precursor [Homo sapiens] | IL17R | 23238208 |
| interleukin 17B receptor isoform 1 precursor; IL-17B receptor; interleukin 17 receptor | IL17RB | 27477074 |
| homolog 1; interleukin 17 receptor homolog; cytokine receptor CRL4; interleukin 17B | | |
| receptor [Homo sapiens]. | | |
| interleukin 17B receptor isoform 2 precursor; IL-17B receptor; interleukin 17 receptor | IL17RB | 27477076 |
| homolog 1; interleukin 17 receptor homolog; cytokine receptor CRL4; interleukin 17B | | |
| receptor [Homo sapiens]. | | |
| interleukin 17 receptor C isoform 1 precursor; interleukin 17 receptor-like [Homo sapiens]. | IL17RC | 24430197 |
| interleukin 17 receptor C isoform 2 precursor; interleukin 17 receptor-like [Homo sapiens]. | IL17RC | 24430195 |
| interleukin 17 receptor C isoform 3 precursor; interleukin 17 receptor-like [Homo sapiens]. | IL17RC | 14249350 |
| interleukin 17 receptor C isoform 4 precursor; interleukin 17 receptor-like [Homo sapiens]. | IL17RC | 24430199 |
| interleukin 17 receptor C isoform 5 precursor; interleukin 17 receptor-like [Homo sapiens]. | IL17RC | 24430201 |
| interleukin 17 receptor D; similar expression to FGF protein [Homo sapiens]. | IL17RD | 24308147 |
| interleukin 17 receptor E isoform 1 [Homo sapiens]. | IL17RE | 24430204 |
| interleukin 17 receptor E isoform 2 [Homo sapiens]. | IL17RE | 24430206 |
| interleukin 17 receptor E isoform 3 [Homo sapiens]. | IL17RE | 21389477 |
| interleukin 17 receptor E isoform 4 [Homo sapiens]. | IL17RE | 24430208 |
| interleukin 17 receptor E isoform 5 [Homo sapiens]. | IL17RE | 24430210 |
| interleukin 18 proprotein; interferon-gamma-inducing factor; interleukin-1 gamma; IL- | IL18 | 4504653 |
| 1 gamma [Homo sapiens]. | | |
| interleukin 18 receptor 1 precursor; IL1 receptor-related protein [Homo sapiens]. | IL18R1 | 4504655 |
| interferon, gamma [Homo sapiens]. | IFNG | 10835171 |
| AntiInflammatory Cytokine Molecules | | |
| colony stimulating factor 2 precursor; sargramostim; granulocyte-macrophage colony | CSF2 | 27437030 |
| stimulating factor; molgramostin [Homo sapiens]. | | |
| colony stimulating factor 3 isoform a precursor; granulocyte colony stimulating factor; | CSF3 | 4503079 |
| pluripoietin; filgrastim; lenograstim [Homo sapiens]. | | |
| colony stimulating factor 3 isoform c; granulocyte colony stimulating factor; | CSF3 | 27437051 |
| pluripoietin; filgrastim; lenograstim [Homo sapiens]. | | |
| colony stimulating factor 3 isoform b; granulocyte colony stimulating factor; | CSF3 | 27437049 |
| pluripoietin; filgrastim; lenograstim [Homo sapiens]. | | |
| colony stimulating factor 3 receptor isoform d precursor; granulocyte colony | CSF3 | 27437045 |
| stimulating factor receptor; CD114 antigen [Homo sapiens]. | | |
| colony stimulating factor 3 receptor isoform b precursor; granulocyte colony | CSF3R | 27437042 |
| stimulating factor receptor; CD114 antigen [Homo sapiens]. | | |
| colony stimulating factor 3 receptor isoform c precursor; granulocyte colony | CSF3R | 24496783 |
| stimulating factor receptor; CD114 antigen [Homo sapiens]. | | |
| colony stimulating factor 3 receptor isoform a precursor; granulocyte colony | CSF3R | 4503081 |
| stimulating factor receptor; CD114 antigen [Homo sapiens]. | | |
| interleukin 1 receptor, type I precursor; interleukin 1 receptor alpha, type I; interleukin | IL1R1 | 4504659 |
| receptor 1; antigen CD121a [Homo sapiens]. | | |
| interleukin 4 isoform 1 precursor; B_cell stimulatory factor 1; lymphocyte stimulatory | IL4 | 4504669 |
| factor 1 [Homo sapiens]. | | |
| interleukin 4 isoform 2 precursor; B_cell stimulatory factor 1; lymphocyte stimulatory | IL4 | 27477092 |
| factor 1 [Homo sapiens]. | | |
| interleukin 4 receptor precursor; CD124 [Homo sapiens]. | IL4R | 4557669 |
| interleukin 6 (interferon, beta 2) [Homo sapiens]. | IL6 | 10834984 |
| interleukin 6 receptor isoform 1 precursor; CD126 antigen; interleukin 6 receptor alpha | IL6R | 4504673 |
| subunit [Homo sapiens]. | | |
| interleukin 6 receptor isoform 2 precursor; CD126 antigen; interleukin 6 receptor alpha | IL6R | 31317249 |
| subunit [Homo sapiens] | | |
| interleukin 10 precursor; cytokine synthesis inhibitory factor [Homo sapiens]. | IL10 | 10835141 |
| interleukin 10 receptor, alpha precursor [Homo sapiens]. | IL10RA | 4504633 |
| interleukin 10 receptor, beta precursor; cytokine receptor family II, member 4; cytokine | IL10RB | 24430215 |
| receptor class-II CRF2-4 [Homo sapiens]. | | |
| interleukin 13 precursor [Homo sapiens]. | IL13 | 26787978 |
| interleukin 13 receptor, alpha 1 precursor; IL13 receptor alpha-1 chain; CD213a1 | IL13RA1 | 4504647 |
| antigen [Homo sapiens]. | | |
| interleukin 13 receptor, alpha 2 precursor; interleukin 13 binding protein; interleukin | IL13RA2 | 10834992 |
| 13 receptor alpha 2 chain; IL-13 receptor [Homo sapiens]. | | |
| interleukin 16 isoform 1 proprotein [Homo sapiens]. | IL16 | 27262655 |
| interleukin 16 isoform 2; lymphocyte chemoattractant factor [Homo sapiens]. | IL16 | 27262657 |
| TGFB inducible early growth response [Homo sapiens]. | TGF-beta | 5032177 |
| Tumor necrosis factor (TNF)-alpha | TNFα | 25952111 |
| Other Cytokine Molecules | | |
| interleukin 19 isoform 1 precursor; melanoma differentiation associated protein-like | IL19 | 30795210 |
| protein [Homo sapiens]. | | |
| interleukin 19 isoform 2 precursor; melanoma differentiation associated protein-like | IL19 | 30795208 |
| protein [Homo sapiens]. | | |
| interleukin 20 precursor; four alpha helix cytokine [Homo sapiens]. | IL20 | 31083166 |
| interleukin 20 receptor, alpha; class II cytokine receptor ZCYTOR7 [Homo sapiens]. | IL20RA | 31083156 |
| interleukin 21 [Homo sapiens]. | IL21 | 11141875 |
| interleukin 21 receptor precursor [Homo sapiens] | IL21R | 31083174 |
| interleukin 21 receptor precursor [Homo sapiens] | IL21R | 31083180 |
| interleukin 21 receptor precursor [Homo sapiens]. | IL21R | 11141869 |
| interleukin 22; interleukin 21; IL-10-related T-cell-derived inducible factor [Homo sapiens] | IL22 | 10092625 |
| interleukin 22 receptor, alpha 1 [Homo sapiens] | IL22RA1 | 31317239 |
| interleukin 22-binding protein isoform 3; class II cytokine receptor; interleukin 22- | IL22RA2 | 31317243 |
| binding protein [Homo sapiens]. | | |
| interleukin 22-binding protein isoform 2; class II cytokine receptor; interleukin 22- | IL22RA2 | 31317241 |
| binding protein [Homo sapiens]. | | |
| interleukin 23, alpha subunit p19 precursor; interleukin 23 p19 subunit; JKA3 induced | IL23A | 7706702 |
| upon T-cell activation [Homo sapiens]. | | |
| interleukin-23 receptor precursor [Homo sapiens]. | IL23R | 24430212 |
| interleukin 24 isoform 1 precursor; melanoma differentiation association protein 7; | IL24 | 5803086 |
| suppression of tumorigenicity 16 (melanoma differentiation) [Homo sapiens]. | | |
| interleukin 24 isoform 2; melanoma differentiation association protein 7; suppression | IL24 | 31317246 |
| of tumorigenicity 16 (melanoma differentiation) [Homo sapiens]. | | induces |
| | | apoptosis |
| interleukin 28A; interferon-lambda 2 [Homo sapiens]. | IL28A | 26024321 |
| interleukin 28B; interferon, lambda 3 [Homo sapiens]. | IL28B | 28144901 |
| interleukin 28 receptor, alpha isoform 1; class II cytokine receptor CRF2/12; interferon | IL28RA | 25014103 |
| lambda, receptor 1; interleukin or cytokine receptor 2; interleukin 28 receptor A | | |
| [Homo sapiens]. | | |
| interleukin 28 receptor, alpha isoform 2; class II cytokine receptor CRF2/12; interferon | IL28RA | 28416909 |
| lambda, receptor 1; interleukin or cytokine receptor 2; interleukin 28 receptor A [Homo sapiens]. | | |
| interleukin 28 receptor, alpha isoform 3; class II cytokine receptor CRF2/12; interferon | IL28RA | 28416911 |
| lambda, receptor 1; interleukin or cytokine receptor 2; interleukin 28 receptor A [Homo sapiens]. | | |
| interleukin 29; interferon, lambda 1 [Homo sapiens]. | IL29 | 26024325 |
| interleukin 30; IL-27 p28 subunit [Homo sapiens]. | IL30 | 28416913 |
| FOXO Family Molecules | | |
| Forkhead box O1A [Homo sapiens]. | FOXO1A | 9257222 |
| Forkhead box O3A; forkhead (Drosophila) homolog (rhabdomyosarcoma) like 1; | FOXO3A | 4503739 |
| forkhead, Drosophila, homolog of, in rhabdomyosarcoma-like 1 [Homo sapiens]. | | |
| JAK/STAT Family Molecules | | |
| janus kinase 1 [Homo sapiens]. | JAK1 | 4504803 |
| Janus kinase 2; tyrosine-protein kinase JAK2 [Homo sapiens]. | JAK2 | 4826776 |
| signal transducer and activator of transcription 1 isoform alpha; signal transducer and | STAT1 | 6274552 |
| activator of transcription-1; signal transducer and activator of transcription 1, 91 kD; | | |
| transcription factor ISGF-3; transcription factor ISGF-3 components p91/p84 [Homo sapiens]. | | |
| signal transducer and activator of transcription 1 isoform beta; signal transducer and | STAT1 | 21536301 |
| activator of transcription-1; signal transducer and activator of transcription 1, 91 kD; | | |
| transcription factor ISGF-3; transcription factor ISGF-3 components p91/p84 [Homo sapiens]. | | |
| signal transducer and activator of transcription 2; signal transducer and activator of | STAT2 | 4885615 |
| transcription 2, 113 kD; interferon alpha induced transcriptional activator [Homo sapiens]. | | |
| signal transducer and activator of transcription 3 isoform 1; acute-phase response | STAT3 | 21618340 |
| factor; DNA-binding protein APRF [Homo sapiens]. | | |
| signal transducer and activator of transcription 3 isoform 2; acute-phase response | STAT3 | 21618338 |
| factor; DNA-binding protein APRF [Homo sapiens]. | | |
| signal transducer and activator of transcription 4 [Homo sapiens]. | STAT4 | 4507255 |
| Signal transducer and activator of transcription 5A; signal transducer and activator of | Stat5a | 21618342 |
| transcription 5 [Homo sapiens]. | | |
| Signal transducer and activator of transcription 5B; signal transducer and activator of | Stat5b | 21618344 |
| transcription 5; transcription factor STAT5B [Homo sapiens]. | | |
| signal transducer and activator of transcription 6; STAT, interleukin4-induced; | STAT6 | 23397678 |
| transcription factor IL-4 STAT [Homo sapiens]. | | |
| Kinase Family Molecules | | |
| Cyclin-dependent kinase 2 isoform 1; cdc2-related protein kinase; cell devision kinase | CDK2 | 16936528 |
| 2; p33 protein kinase [Homo sapiens]. | | |
| Cyclin-dependent kinase 2 isoform 2; cdc2-related protein kinase; cell devision kinase | CDK2 | 16936530 |
| 2; p33 protein kinase [Homo sapiens]. | | |
| Cyclin-dependent kinase 4 isoform 1; cell division kinase 4; melanoma cutaneous | CDK4 | 4502735 |
| malignant, 3 [Homo sapiens]. | | |
| Cyclin-dependent kinase 4 isoform 2; cell division kinase 4; melanoma cutaneous | CDK4 | 16936533 |
| malignant, 3 [Homo sapiens]. | | |
| Cyclin-dependent kinase inhibitor 1A; melanoma differentiation associated protein 6; | CDKN1A | 11386203 |
| CDK-interaction protein 1; wild-type p53-activated fragment 1; DNA synthesis | | |
| inhibitor [Homo sapiens]. | | |
| Cyclin-dependent kinase inhibitor 1A; melanoma differentiation associated protein 6; | CDKN1A | 17978495 |
| CDK-interaction protein 1; wild-type p53-activated fragment 1; DNA synthesis | | |
| inhibitor [Homo sapiens]. | | |
| Cyclin-dependent kinase inhibitor 1B (p27, Kip1) [Homo sapiens]. | CDKN1B | 18656929 |
| cyclin-dependent kinase inhibitor 2A isoform 1; multiple tumor suppressor 1; cyclin- | CDKN2A | 4502749 |
| dependent kinase inhibitor p16; cyclin-dependent kinase inhibitor p12, p16INK4a | | |
| alternatively spliced form; cell cycle negative regulator beta; CDK4 inhibitor p16- | | |
| INK4 [Homo sapiens]. | | |
| Cyclin-dependent kinase inhibitor 2A isoform 2; multiple tumor suppressor 1; cyclin- | CDKN2A | 17738296 |
| dependent kinase inhibitor p16; cyclin-dependent kinase inhibitor p12, p16INK4a | | |
| alternatively spliced form; cell cycle negative regulator beta; CDK4 inhibitor p16- | | |
| INK4 [Homo sapiens]. | | |
| Cyclin-dependent kinase inhibitor 2A isoform 3; multiple tumor suppressor 1; cyclin- | CDKN2A | 17738298 |
| dependent kinase inhibitor p16; cyclin-dependent kinase inhibitor p12, p16INK4a | | |
| alternatively spliced form; cell cycle negative regulator beta; CDK4 inhibitor p16- | | |
| INK4 [Homo sapiens]. | | |
| mitogen-activated protein kinase kinase 1; protein kinase, mitogen-activated, kinase 1 | MAP2K1 | 5579478 |
| (MAP kinase kinase 1) [Homo sapiens]. | | |
| mitogen-activated protein kinase kinase 1 interacting protein 1; MEK partner 1; MEK | MAP2K1IP1 | 11496277 |
| binding partner 1 [Homo sapiens]. | | |
| mitogen-activated protein kinase kinase 2; mitogen-activated protein kinase kinase 2, | MAP2K2 | 13489054 |
| p45; MAP kinase kinase 2; MAPK/ERK kinase 2; dual specificity mitogen-activated | | |
| protein kinase kinase 2; ERK activator kinase 2 [Homo sapiens]. | | |
| mitogen-activated protein kinase kinase 3 isoform A; MAP kinase kinase 3; | MAP2K3 | 21618347 |
| MAPK/ERK kinase 3; dual specificity mitogen activated protein kinase kinase 3 | | |
| [Homo sapiens]. | | |
| mitogen-activated protein kinase kinase 3 isoform B; MAP kinase kinase 3; | MAP2K3 | 21618349 |
| MAPK/ERK kinase 3; dual specificity mitogen activated protein kinase kinase 3 | | |
| [Homo sapiens]. | | |
| mitogen-activated protein kinase kinase 3 isoform C; MAP kinase kinase 3; | MAP2K3 | 21618351 |
| MAPK/ERK kinase 3; dual specificity mitogen activated protein kinase kinase 3 | | |
| [Homo sapiens]. | | |
| mitogen-activated protein kinase kinase 4; dual specificity mitogen-activated protein | MAP2K4 | 4506889 |
| kinase kinase 4; MAP kinase kinase 4; c-Jun N-terminal kinase kinase 1; JNK | | |
| activating kinase 1; SAPK/ERK kinase 1; MAPK/ERK kinase 4; JNK-activated kinase | | |
| 1 [Homo sapiens]. | | |
| mitogen-activated protein kinase kinase 5 isoform A; dual specificity mitogen- | MAP2K5 | 21729895 |
| activated protein kinase kinase 5; MAP kinase kinase MEK5b; MAPK/ERK kinase 5 | | |
| [Homo sapiens]. | | |
| mitogen-activated protein kinase kinase 5 isoform B; dual specificity mitogen-activated | MAP2K5 | 4506101 |
| protein kinase kinase 5; MAP kinase kinase MEK5b; MAPK/ERK kinase 5 [Homo sapiens]. | | |
| mitogen-activated protein kinase kinase 5 isoform C; dual specificity mitogen-activated | MAP2K5 | 21729897 |
| protein kinase kinase 5; MAP kinase kinase MEK5b; MAPK/ERK kinase 5 [Homo sapiens]. | | |
| mitogen-activated protein kinase kinase 5 isoform D; dual specificity mitogen- | MAP2K5 | 21729899 |
| activated protein kinase kinase 5; MAP kinase kinase MEK5b; MAPK/ERK kinase 5 | | |
| [Homo sapiens]. | | |
| mitogen-activated protein kinase kinase 6 isoform 1; protein kinase, mitogen-activated, | MAP2K6 | 14589900 |
| kinase 6 (MAP kinase kinase 6) [Homo sapiens]. | | |
| mitogen-activated protein kinase kinase 6 isoform 2; protein kinase, mitogen-activated, | MAP2K6 | 14589902 |
| kinase 6 (MAP kinase kinase 6) [Homo sapiens]. | | |
| mitogen-activated protein kinase kinase 7 isoform A; dual specificity mitogen- | MAP2K7 | 21735542 |
| activated protein kinase kinase 7; c-Jun N-terminal kinase kinase 2; MAP kinase | | |
| kinase 7; JNK-activating kinase 2; JNK kinase 2 [Homo sapiens]. | | |
| mitogen-activated protein kinase kinase 7 isoform B; dual specificity mitogen-activated | MAP2K7 | 21735544 |
| protein kinase kinase 7; c-Jun N-terminal kinase kinase 2; MAP kinase kinase 7; JNK- | | |
| activating kinase 2; JNK kinase 2 [Homo sapiens]. | | |
| mitogen-activated protein kinase kinase 7 isoform C; dual specificity mitogen-activated | MAP2K7 | 21735546 |
| protein kinase kinase 7; c-Jun N-terminal kinase kinase 2; MAP kinase kinase 7; JNK- | | |
| activating kinase 2; JNK kinase 2 [Homo sapiens]. | | |
| mitogen-activated protein kinase kinase kinase 10; mixed lineage kinase 2; MKN28 | MAP3K10 | 21735550 |
| kinase; MKN28 derived nonreceptor_type serine/threonine kinase [Homo sapiens]. | | |
| mitogen-activated protein kinase kinase kinase 11; mixed lineage kinase 3; SH3 | MAP3K11 | 4505195 |
| domain-containing proline-rich kinase; protein-tyrosine kinase PTK1 [Homo sapiens]. | | |
| mitogen-activated protein kinase kinase kinase 12; leucine zipper protein kinase; | MAP3K12 | 21735552 |
| zipper protein kinase; protein kinase MUK; dual leucine zipper kinase DLK [Homo sapiens]. | | |
| mitogen-activated protein kinase kinase kinase 13; leucine zipper-bearing kinase | MAP3K13 | 4758696 |
| [Homo sapiens]. | | |
| mitogen-activated protein kinase kinase kinase 14; serine/threonine protein-kinase | MAP3K14 | 4505397 |
| [Homo sapiens]. | | |
| mitogen-activated protein kinase kinase kinase 2; MAP/ERK kinase kinase 2; | MAP3K2 | 21735556 |
| MAPK/ERK kinase kinase 2; MEK kinase 2 [Homo sapiens]. | | |
| MAP/ERK kinase kinase 4 isoform a; SSK2/SSK22 MAP kinase kinase kinase, yeast, | MAP3K4 | 5803088 |
| homolog of [Homo sapiens]. | | |
| MAP/ERK kinase kinase 4 isoform b; SSK2/SSK22 MAP kinase kinase kinase, yeast, | MAP3K4 | 6031180 |
| homolog of [Homo sapiens]. | | |
| MAP/ERK kinase kinase 5; apoptosis signal regulating kinase [Homo sapiens]. | MAP3K5 | 5174547 |
| mitogen-activated protein kinase kinase kinase 6 isoform 1 [Homo sapiens]. | MAP3K6 | 24497522 |
| mitogen-activated protein kinase kinase kinase 6 isoform 2 [Homo sapiens]. | MAP3K6 | 24497524 |
| mitogen-activated protein kinase kinase kinase 7 isoform B; transforming growth | MAP3K7 | 21735562 |
| factor-beta-activated kinase 1; TGF-beta activated kinase 1 [Homo sapiens]. | | |
| mitogen-activated protein kinase kinase kinase 7 isoform C; transforming growth | MAP3K7 | 21735564 |
| factor-beta-activated kinase 1; TGF-beta activated kinase 1 [Homo sapiens]. | | |
| mitogen-activated protein kinase kinase kinase 7 isoform D; transforming growth | MAP3K7 | 21735566 |
| factor-beta-activated kinase 1; TGF-beta activated kinase 1 [Homo sapiens]. | | |
| mitogen-activated protein kinase kinase kinase 7 isoform A; transforming growth | MAP3K7 | 4507361 |
| factor-beta-activated kinase 1; TGF-beta activated kinase 1 [Homo sapiens]. | | |
| mitogen-activated protein kinase kinase kinase 7 interacting protein 1; TAK1-binding | MAP3K7IP1 | 5174703 |
| protein 1; transforming growth factor beta-activated kinase-binding protein 1 [Homo sapiens]. | | |
| mitogen-activated protein kinase kinase kinase 8; cot (cancer Osaka thyroid) oncogene; | MAP3K8 | 22035598 |
| Cancer Osaka thyroid oncogene; Ewing sarcoma transformant; proto-oncogene | | |
| serine/threoine protein kinase; tumor progression locus-2 [Homo sapiens]. | | |
| mitogen-activated protein kinase kinase kinase kinase 1 [Homo sapiens]. | MAP4K1 | 6005810 |
| mitogen-activated protein kinase kinase kinase kinase 2; Rab8 interacting protein; | MAP4K2 | 22035600 |
| germinal centre kinase (GC kinase); B lymphocyte serine/threonine protein kinase | | |
| [Homo sapiens]. | | |
| mitogen-activated protein kinase kinase kinase kinase 3; germinal center kinase-related | MAP4K3 | 15451902 |
| protein kinase; germinal center kinase-like kinase [Homo sapiens]. | | |
| mitogen-activated protein kinase kinase kinase kinase 4 isoform 1; HPK/GCK-like | MAP4K4 | 22035602 |
| kinase [Homo sapiens]. | | |
| mitogen-activated protein kinase kinase kinase kinase 4 isoform 2; HPK/GCK-like | MAP4K4 | 22035604 |
| kinase [Homo sapiens]. | | |
| mitogen-activated protein kinase kinase kinase kinase 4 isoform 3; HPK/GCK-like | MAP4K4 | 22035606 |
| kinase [Homo sapiens]. | | |
| mitogen-activated protein kinase kinase kinase kinase 5; kinase homologous to | MAP4K5 | 14589909 |
| SPS1/STE20 [Homo sapiens]. | | |
| mitogen-activated protein kinase 1; extracellular signal-regulated kinase 2; protein | MAPK1 | 20986531 |
| tyrosine kinase ERK2; mitogen-activated protein kinase 2 [Homo sapiens]. | | |
| mitogen-activated protein kinase 1; extracellular signal-regulated kinase 2; protein | MAPK1 | 20986529 |
| tyrosine kinase ERK2; mitogen-activated protein kinase 2 [Homo sapiens]. | | |
| mitogen-activated protein kinase 10 isoform 1; JNK3 alpha protein kinase; stress | MAPK10 | 4506081 |
| activated protein kinase beta; MAP kinase; c-Jun kinase 3; c-Jun N-terminal kinase 3; | | |
| stress activated protein kinase JNK3 [Homo sapiens]. | | |
| mitogen-activated protein kinase 10 isoform 2; JNK3 alpha protein kinase; stress | MAPK10 | 20986510 |
| activated protein kinase beta; MAP kinase; c-June kinase 3; c-Jun N-terminal kinase 3; | | |
| stress activated protein kinase JNK3 [Homo sapiens]. | | |
| mitogen-activated protein kinase 10 isoform 3; JNK3 alpha protein kinase; stress | MAPK10 | 20986506 |
| activated protein kinase beta; MAP kinase; c-Jun kinase 3; c-Jun N-terminal kinase 3; | | |
| stress activated protein kinase JNK3 [Homo sapiens]. | | |
| mitogen-activated protein kinase 10 isoform 4; JNK3 alpha protein kinase; stress | MAPK10 | 20986508 |
| activated protein kinase beta; MAP kinase; c-Jun kinase 3; c-Jun N-terminal kinase 3; | | |
| stress activated protein kinase JNK3 [Homo sapiens]. | | |
| mitogen-activated protein kinase 11; mitogen-activated protein kinase p38beta; stress- | MAPK11 | 20128774 |
| activated protein kinase-2; stress-activated protein kinase-2b; mitogen-activated | | |
| protein kinase p38-2 [Homo sapiens]. | | |
| mitogen-activated protein kinase 11; mitogen-activated protein kinase p38beta; stress- | MAPK11 | 20986526 |
| activated protein kinase-2; stress activated protein kinase-2b; mitogen-activated | | |
| protein kinase p38-2 [Homo sapiens]. | | |
| mitogen-activated protein kinase 12; p38gamma; stress-activated protein kinase 3; | MAPK12 | 4506785 |
| mitogen-activated protein kinase 3 [Homo sapiens]. | | |
| mitogen-activated protein kinase 13; mitogen-activated protein kinase p38 delta; stress- | MAPK13 | 4506085 |
| activated protein kinase 4 [Homo sapiens]. | | |
| mitogen-activated protein kinase 14 isoform 1; cytokine suppressive anti-inflammatory | MAPK14 | 4503069 |
| drug binding protein; Csaids binding protein; MAP kinase Mxi2; p38 mitogen | | |
| activated protein kinase; p38 MAP kinase; p38alpha Exip; stress-activated protein | | |
| kinase 2A; MAX-interacting protein 2 [Homo sapiens]. | | |
| mitogen-activated protein kinase 14 isoform 2; cytokine suppressive anti-inflammatory | MAPK14 | 20986512 |
| drug binding protein; Csaids binding protein; MAP kinase Mxi2; p38 mitogen | | |
| activated protein kinase; p38 MAP kinase; p38alpha Exip; stress-activated protein | | |
| kinase 2A; MAX-interacting protein 2 [Homo sapiens]. | | |
| mitogen-activated protein kinase 14 isoform 3; cytokine suppressive anti-inflammatory | MAPK14 | 20986514 |
| drug binding protein; Csaids binding protein; MAP kinase Mxi2; p38 mitogen | | |
| activated protein kinase; p38 MAP kinase; p38alpha Exip; stress-activated protein | | |
| kinase 2A; MAX-interacting protein 2 [Homo sapiens]. | | |
| mitogen-activated protein kinase 14 isoform 4; cytokine suppressive anti-inflammatory | MAPK14 | 20986516 |
| drug binding protein; Csaids binding protein; MAP kinase Mxi2; p38 mitogen | | |
| activated protein kinase; p38 MAP kinase; p38alpha Exip; stress-activated protein | | |
| kinase 2A; MAX-interacting protein 2 [Homo sapiens]. | | |
| mitogen-activated protein kinase 4; Erk3-related; protein kinase, mitogen-activated 4 | MAPK4 | 4506089 |
| (MAP kinase 4; p63) [Homo sapiens]. | | |
| mitogen-activated protein kinase 6; protein kinase, mitogen-activated 5; protein kinase, | MAPK6 | 4506091 |
| mitogen-activated 6; extracellular signal-regulated kinase, p97; MAP kinase isoform | | |
| p97; extracellular signal-regulated kinase 3 [Homo sapiens]. | | |
| mitogen-activated protein kinase 7 isoform 1; BMK1 kinase; extracellular-signal- | MAPK7 | 20986497 |
| regulated kinase 5 [Homo sapiens]. | | |
| mitogen-activated protein kinase 7 isoform 1; BMK1 kinase; extracellular-signal- | MAPK7 | 20986501 |
| regulated kinase 5 [Homo sapiens]. | | |
| mitogen-activated protein kinase 7 isoform 1; BMK1 kinase; extracellular-signal- | MAPK7 | 20986503 |
| regulated kinase 5 [Homo sapiens]. | | |
| mitogen-activated protein kinase 7 isoform 2; BMK1 kinase; extracellular-signal- | MAPK7 | 20986499 |
| regulated kinase 5 [Homo sapiens]. | | |
| mitogen-activated protein kinase 8 isoform 1; c-Jun N-terminalmitogen-activated | MAPK8 | 20986523 |
| protein kinase 8 isoform 1; c-Jun N-terminal kinase 1; JNK1 alpha protein kinase; | | |
| protein kinase JNK1; JNK1 beta protein kinase; stress-activated protein kinase JNK1 | | |
| [Homo sapiens]. | | |
| mitogen-activated protein kinase 8 isoform 2; c-Jun N-terminal kinase 1; JNK1 alpha | MAPK8 | 4506095 |
| protein kinase; protein kinase JNK1; JNK1 beta protein kinase; stress-activated protein | | |
| kinase JNK1 [Homo sapiens]. | | |
| mitogen-activated protein kinase 8 isoform 3; c-Jun N-terminal kinase 1; JNK1 alpha | MAPK8 | 20986519 |
| protein kinase; protein kinase JNK1; JNK1 beta protein kinase; stress-activated protein | | |
| kinase JNK1 [Homo sapiens]. | | |
| mitogen-activated protein kinase 8 isoform 4; c-Jun N-terminal kinase 1; JNK1 alpha | MAPK8 | 20986521 |
| protein kinase; protein kinase JNK1; JNK1 beta protein kinase; stress-activated protein | | |
| kinase JNK1 [Homo sapiens]. | | |
| mitogen-activated protein kinase 9 isoform 1; Jun kinase; MAP kinase 9; c-Jun kinase | MAPK9 | 21237736 |
| 2; c-Jun N-terminal kinase 2; stress-activated protein kinase JNK2 [Homo sapiens]. | | |
| mitogen-activated protein kinase 9 isoform 2; Jun kinase; MAP kinase 9; c-Jun kinase | MAPK9 | 21237739 |
| 2; c-Jun N-terminal kinase 2; stress-activated protein kinase JNK2 [Homo sapiens]. | | |
| mitogen-activated protein kinase 9 isoform 3; Jun kinase; MAP kinase 9; c-Jun kinase | MAPK9 | 21237742 |
| 2; c-Jun N-terminal kinase 2; stress-activated protein kinase JNK2 [Homo sapiens]. | | |
| mitogen-activated protein kinase 9 isoform 4; Jun kinase; MAP kinase 9; c-Jun kinase | MAPK9 | 21237745 |
| 2; c-Jun N-terminal kinase 2; stress-activated protein kinase JNK2 [Homo sapiens]. | | |
| mitogen-activated protein kinase-activated protein kinase 2 isoform 1 [Homo sapiens]. | MAPKAPK2 | 10863901 |
| mitogen-activated protein kinase-activated protein kinase 2 isoform 2 [Homo sapiens]. | MAPKAPK2 | 32481209 |
| mitogen-activated protein kinase-activated protein kinase 3; MAPKAP kinase 3 [Homo sapiens]. | MAPKAPK3 | 4758700 |
| mitogen-activated protein kinase-activated protein kinase 5 isoform 2; p38- | MAPKAPK5 | 21237768 |
| regulated/activated protein kinase [Homo sapiens]. | | |
| Other Apoptosis Related Molecules | | |
| Apoptotic protease activating factor isoform a; apoptotic protease activating factor 1 | APAF1 | 7108333 |
| [Homo sapiens]. | | |
| Apoptotic protease activating factor isoform b; apoptotic protease activating factor 1 | APAF1 | 4502123 |
| [Homo sapiens]. | | |
| Apoptotic protease activating factor isoform c; apoptotic protease activating factor 1 | APAF1 | 32483359 |
| [Homo sapiens]. | | |
| Apoptotic protease activating factor isoform d; apoptotic protease activating factor 1 | APAF1 | 32483361 |
| [Homo sapiens]. | | |
| Apoptotic protease activating factor isoform e; apoptotic protease activating factor 1 | APAF1 | 32483363 |
| [Homo sapiens]. | | |
| activating transcription factor 1 [Homo sapiens]. | ATF1 | 4885073 |
| caspase recruitment domain protein 10; CARD-containing MAGUK 3 protein; Bcl10 | CARD10 | 17977658 |
| binding protein and activator of NFKB [Homo sapiens]. | | |
| caspase recruitment domain family, member 11; card-maguk protein 1; bcl10- | CARD11 | 16507952 |
| interacting maguk protein 3 [Homo sapiens]. | | |
| caspase recruitment domain protein 12; ICE-protease activating factor [Homo sapiens]. | CARD12 | 20127598 |
| caspase recruitment domain protein 14 isoform 1; CARD-containing MAGUK 2 | CARD14 | 13129124 |
| protein; bcl10-interacting maguk protein 2; card-maguk protein 2 [Homo sapiens]. | | |
| caspase recruitment domain protein 14 isoform 2; CARD-containing MAGUK 2 | CARD14 | 16507955 |
| protein; bcl10-interacting maguk protein 2; card-maguk protein 2 [Homo sapiens]. | | |
| caspase recruitment domain family, member 4; caspase recruitment domain 4 [Homo sapiens]. | CARD4 | 5174617 |
| caspase recruitment domain family, member 6; caspase recruitment domain protein 6 | CARD6 | 16554564 |
| [Homo sapiens]. | | |
| caspase recruitment domain protein 9 isoform 1 [Homo sapiens]. | CARD9 | 16554560 |
| caspase recruitment domain protein 9 isoform 2 [Homo sapiens]. | CARD9 | 16554562 |
| caspase recruitment domain protein 9 isoform 3 [Homo sapiens]. | CARD9 | 16554558 |
| cyclin B1; G2/mitotic-specific cyclin B1 [Homo sapiens]. | CCNB1 | 14327896 |
| Apoptosis (APO-1) antigen ligand 1; CD95 ligand [Homo sapiens]. | CD95-ligand | 4557329 |
| Cell division cycle 25A; protein-tyrosine-phosphatase | Cdc25A | 4502705 |
| Cell division control protein 2 homolog (p34 protein kinase) (Cyclin-dependent kinase | CDK1 | 115922 |
| 1) (CDK1). | | |
| CCAAT/enhancer binding protein beta; interleukin 6-dependent DNA-binding protein; | CEBPB | 28872796 |
| nuclear factor of interleukin 6; transcription factor 5; liver-enriched transcriptional | | |
| activator protein [Homo sapiens] | | |
| CASP8 and FADD-like apoptosis regulator; FADD-like anti-apoptotic molecule; | CFLAR | 21361769 |
| Inhibitor of FLICE; Caspase-related inducer of apoptosis; Caspase homolog; Caspase- | | |
| like apoptosis regulatory protein [Homo sapiens]. | | |
| Casein kinase 1, alpha 1; down-regulated in lung cancer [Homo sapiens]. | CK1 | 19923746 |
| c-Myc oncogene [Homo sapiens] | c-Myc | 34816 |
| CREB binding protein [Homo sapiens]. | CREBBP | 4758056 |
| Cyclin A [Homo sapiens] | Cyclin A | 4502613 |
| Cyclin D1; G1/S-specific cyclin D1; B-cell CLL/lymphoma 1 [Homo sapiens] | CyclinD1 | 16950655 |
| Cyclin D2; G1/S-specific cyclin D2 [Homo sapiens] | CyclinD2 | 4502617 |
| Cyclin E1 isoform 1; Cyclin E; Cyclin Es; Cyclin Et [Homo sapiens]. | CyclinE | 17318559 |
| death associated protein 3; mitochondrial 28S ribosomal protein S29 [Homo sapiens]. | DAP3 | 16905526 |
| death-associated protein kinase 2 [Homo sapiens]. | DAPK2 | 14670383 |
| death effector filament-forming Ced-4-like apoptosis protein isoform 1; caspase | DEFCAP | 14719829 |
| recruitment domain protein 7; NAC-alpha/beta/gamma/delta; neuronal apoptosis | | |
| inhibitor protein 1 [Homo sapiens]. | | |
| death effector filament-forming Ced-4-like apoptosis protein isoform 2; caspase | DEFCAP | 14719831 |
| recruitment domain protein 7; NAC-alpha/beta/gamma/delta [Homo sapiens]. | | |
| death effector filament-forming Ced-4-like apoptosis protein isoform 2; caspase | DEFCAP | 7662386 |
| recruitment domain protein 7; NAC-alpha/beta/gamma/delta [Homo sapiens]. | | |
| death effector filament-forming Ced-4-like apoptosis protein isoform 3; caspase | DEFCAP | 14719833 |
| recruitment domain protein 7; NAC-alpha/beta/gamma/delta [Homo sapiens]. | | |
| death effector filament-forming Ced-4-like apoptosis protein isoform 4; caspase | DEFCAP | 14719835 |
| recruitment domain protein 7; NAC-alpha/beta/gamma/delta [Homo sapiens]. | | |
| E2F transcription factor 1; retinoblastoma-associated protein 1 [Homo sapiens]. | E2F | 12669911 |
| v-erb-b2 erythroblastic leukemia viral oncogene homolog 2, neuro/glioblastoma | ERBB2 | 4758298 |
| derived oncogene homolog; Avian erythroblastic leukemia viral (v-erb-b2) oncogene | | |
| homolog 2; v-erb-b2 avian erythroblastic leukemia viral oncogene homolog 2 | | |
| (neuro/glioblastoma derived oncogene homolog) [Homo sapiens]. | | |
| v-erb-b2 erythroblastic leukemia viral oncogene homolog 3; Transformation gene | ERBB3 | 4503597 |
| ERBB-3; v-erb-b2 avian erythroblastic leukemia viral oncogene homolog 3 [Homo sapiens]. | | |
| v-erb-a erythroblastic leukemia viral oncogene homolog 4; avian erythroblastic | ERBB4 | 4885215 |
| leukemia viral (v-erb-b2) oncogene homolog 4; v-erb-a avian erythroblastic leukemia | | |
| viral oncogene homolog-like 4 [Homo sapiens]. | | |
| Growth arrest and DNA-damage-inducible protein GADD153 | Gadd153 | 252002 |
| Growth arrest and DNA-damage-inducible, alpha; DNA-damage-inducible transcript | Gadd45A | 4503287 |
| 1; DNA damage-inducible transcript-1; DNA damage-inducible transcript 1 [Homo sapiens]. | | |
| Growth arrest and DNA-damage-inducible, beta; myeloid differentiation primary | Gadd45B | 9945332 |
| response; myeloid differentiation primary response gene [Homo sapiens]. | | |
| hypoxia-inducible factor 1, alpha subunit isoform 1; ARNT interacting protein; | HIF1A | 4504385 |
| member of PAS superfamily 1 [Homo sapiens]. | | |
| hypoxia-inducible factor 1, alpha subunit isoform 2; ARNT interacting protein; | HIF1A | 31077211 |
| member of PAS superfamily 1 [Homo sapiens]. | | |
| hypoxia-inducible factor 1, alpha subunit inhibitor, factor inhibiting HIF1 [Homo sapiens]. | HIF1AN | 8923569 |
| hypoxia-inducible factor-3 alpha isoform a; inhibitory PAS domain protein [Homo sapiens]. | HIF3A | 23065535 |
| hypoxia-inducible factor-3 alpha isoform b; inhibitory PAS domain protein [Homo sapiens]. | HIF3A | 23065532 |
| hypoxia-inducible factor-3 alpha isoform c; inhibitory PAS domain protein [Homo sapiens]. | HIF3A | 23065538 |
| hypoxia-inducible factor-3 alpha isoform d; inhibitory PAS domain protein [Homo sapiens]. | HIF3A | 23065541 |
| MAP/ERK kinase kinase 3 [Homo sapiens]. | hMEKK3 | 4505153 |
| harakiri; activator of apoptosis Hrk; harakiri, BCL2-interacting protein (contains only | HRK | 4504493 |
| BH3 domain); BCL2-interacting protein [Homo sapiens]. | | |
| Heat shock 70 kDa protein 5 (glucose-regulated protein, 78 kDa); BiP; heat shock 70 kD | HSPA5 | 16507237 |
| protein 5 (glucose-regulated protein, 78 kD); Heat-shock 70 kD protein-5 (glucose- | | |
| regulated protein, 78 kD) [Homo sapiens]. | | |
| interferon, gamma [Homo sapiens]. | IFNG | 10835171 |
| interferon gamma receptor 1; Immune interferon, receptor for [Homo sapiens]. | IFNGR1 | 4557880 |
| interferon gamma receptor 2 (interferon gamma transducer 1); interferon gamma | IFNGR2 | 5031783 |
| receptor accessory factor-1; interferon-gamma receptor beta chain precursor [Homo sapiens]. | | |
| interleukin 18 proprotein; interferon-gamma-inducing factor; interleukin-1 gamma, IL- | IL18 | 4504653 |
| 1 gamma [Homo sapiens]. | | |
| v-jun avian sarcoma virus 17 oncogene homolog; Jun activation domain binding | JUN | 4758616 |
| protein; activator protein 1; enhancer-binding protein AP1 [Homo sapiens]. | | |
| c-K-ras2 protein isoform a; Kirsten rat sarcoma-2 viral (v-Ki-ras2) oncogene homolog; | KRAS2 | 15718763 |
| transforming protein p21; c-Kirsten-ras protein; K-ras p21 protein; oncogene KRAS2; | | |
| c-K-ras protein; PR310 c-K-ras oncogene; cellular c-Ki-ras2 proto-oncogene [Homo sapiens]. | | |
| c-K-ras2 protein isoform b; Kirsten rat sarcoma-2 viral (v-Ki-ras2) oncogene homolog; | KRAS2 | 15718761 |
| transforming protein p21; c-Kirsten-ras protein; K-ras p21 protein; oncogene KRAS2; | | |
| c-K-ras protein; PR310 c-K-ras oncogene; cellular c-Ki-ras2 proto-oncogene [Homo sapiens]. | | |
| MAD, mothers against decapentaplegic homolog 1; MAD (mothers against | MADH1 | 5174509 |
| decapentaplegic, Drosophila) homolog 1; Mothers against decapentaplegic, | | |
| Drosophila, homolog of, 1 [Homo sapiens]. | | |
| MAD, mothers against decapentaplegic homolog 2; MAD (mothers against | MADH2 | 5174511 |
| decapentaplegic, Drosophila) homolog 2; Mothers against decapentaplegic, | | |
| Drosophila, homolog of, 2 [Homo sapiens]. | | |
| MAD, mothers against decapentaplegic homolog 3; MAD (mothers against | MADH3 | 5174513 |
| decapentaplegic, Drosophila) homolog 9; Mothers against decapentaplegic, drosophila, | | |
| homolog of, 3 [Homo sapiens]. | | |
| MAD, mothers against decapentaplegic homolog 4; MAD (mothers against | MADH4 | 4885457 |
| decapentaplegic, Drosophila) homolog 4; Mothers against decapentaplegic, | | |
| Drosophila, homolog of, 4 [Homo sapiens]. | | |
| MAD, mothers against decapentaplegic homolog 5; Dwfc; MAD (mothers against | MADH5 | 5174515 |
| decapentaplegic, Drosophila) homolog 5; Mothers against decapentaplegic, drosophila, | | |
| homolog of, 5 [Homo sapiens]. | | |
| MAD, mothers against decapentaplegic homolog 6; Mothers against decapentaplegic, | MADH6 | 19923323 |
| drosophila, homolog of, 6; MAD (mothers against decapentaplegic, Drosophila) | | |
| homolog 6 [Homo sapiens]. | | |
| MAD, mothers against decapentaplegic homolog 7; MAD (mothers against | MADH7 | 5174517 |
| decapentaplegic, Drosophila) homolog 7; Mothers against decapentaplegic, drosophila, | | |
| homolog of, 7 [Homo sapiens]. | | |
| MAD, mothers against decapentaplegic homolog 9; MAD (mothers against | MADH9 | 5174519 |
| decapentaplegic, Drosophila) homolog 9; Mothers against decapentaplegic, drosophila, | | |
| homolog of, 9 [Homo sapiens]. | | |
| MAD, mothers against decapentaplegic homolog (Drosophila) interacting protein, | MADHIP | 6552339 |
| receptor activation anchor isoform 1; Smad anchor for receptor activation; MAD | | |
| (mothers against decapentaplegic, Drosophila) homolog interacting protein, receptor | | |
| activation anchor [Homo sapiens]. | | |
| MAD, mothers against decapentaplegic homolog (Drosophila) interacting protein, | MADHIP | 6552337 |
| receptor activation anchor isoform 2; Smad anchor for receptor activation; MAD | | |
| (mothers against decapentaplegic, Drosophila) homolog interacting protein, receptor | | |
| activation anchor [Homo sapiens]. | | |
| MAD, mothers against decapentaplegic homolog (Drosophila) interacting protein, | MADHIP | 4759060 |
| receptor activation anchor isoform 3; Smad anchor for receptor activation; MAD | | |
| (mothers against | | |
| Modulator of apoptosis 1; MAP-1 protein; paraneoplastic antigen like 4 [Homo sapiens]. | MOAP1 | 19923584 |
| 3-phosphoinositide dependent protein kinase-1; PkB kinase [Homo sapiens]. | PDPK1 | 4505695 |
| Serine/threonine protein kinase; Murine thymoma viral (v-akt) oncogene homolog-1 | PKB/Akt | 4885061 |
| [Homo sapiens]. | | |
| pleiomorphic adenoma gene-like 2; C2H2-type zinc finger protein [Homo sapiens]. | PLAGL2 | 4505859 |
| mitogen-activated protein kinase-activated protein kinase 5 isoform 1; p38- | PRK | 21237765 |
| regulated/activated protein kinase [Homo sapiens]. | | |
| protease, serine, 25 isoform 1 preproprotein; HtrA-like serine protease; high | PRSS25 | 7019477 |
| temperature requirement protein A2; Omi stress-regulated endoprotease [Homo sapiens]. | | |
| protease, serine, 25 isoform 2; HtrA-like serine protease; high temperature requirement | PRSS25 | 21614538 |
| protein A2; Omi stress-regulated endoprotease [Homo sapiens]. | | |
| Phosphatase and tensin homolog; mutated in multiple advanced cancers 1 [Homo sapiens]. | PTEN | 4506249 |
| v-raf-1 murine leukemia viral oncogene homolog 1; Oncogene RAF1; raf proto- | RAF1 | 4506401 |
| oncogene serine/threonine protein kinase [Homo sapiens]. | | |
| receptor (TNFRSF)-interacting serine-threonine kinase 1; receptor interacting protein | RIPK1 | 4506539 |
| [Homo sapiens]. | | |
| receptor-interacting serine-threonine kinase 2; receptor interacting protein 2 [Homo sapiens]. | RIPK2 | 4506537 |
| receptor-interacting serine-threonine kinase 3; receptor-interacting protein 3 [Homo sapiens]. | RIPK3 | 5803143 |
| Activated RNA polymerase II transcriptional coactivator p15 (PC4) (p14). | RPO2TC1 | 1709514 |
| second mitochondria-derived activator of caspase isoform Smac-alpha, precursor; | SMAC | 9845297 |
| direct IAP-binding protein with low pI; mitochondrial Smac protein [Homo sapiens]. | | |
| second mitochondria-derived activator of caspase isoform Smac-beta; direct IAP- | SMAC | 21070978 |
| binding protein with low pI; mitochondrial Smac protein [Homo sapiens]. | | |
| second mitochondria-derived activator of caspase isoform Smac-delta, precursor; direct | SMAC | 21070976 |
| IAP-binding protein with low pI; mitochondrial Smac protein [Homo sapiens]. | | |
| Superoxide dismutase 2, mitochondrial [Homo sapiens]. | SOD2 | 10835187 |
| TRIAD3 protein; zinc finger protein inhibiting NF-kappaB; ubiquitin conjugating | TRIAD3 | 21536434 |
| enzyme 7 interacting protein 1 [Homo sapiens]. | | |
| transducer of ERBB2, 1; transducer of erbB-2 [Homo sapiens]. | TOB1 | 5032187 |
| Tumor protein p53 [Homo sapiens] | TP53 | 8400738 |

We claim:

1-71. (canceled)

72. A composition comprising a double-stranded, short interfering RNA (siRNA) agent which inhibits expression of an apoptosis-related gene.

73. A composition comprising a double-stranded, short interfering RNA (siRNA) agent which inhibits expression of a proinflammatory cytokine.

74. The composition of claim 72, wherein said apoptosis-related gene is an anti-apoptotic gene or a pro-apoptotic gene.

75. The composition of claim 72, wherein said agent is an RNA which is homologous to an apoptosis-related gene, or a fragment thereof.

76. The composition of claim 73, wherein said agent is an RNA which is homologous to proinflammatory cytokine, or a fragment thereof.

77. The composition of claim 76, wherein the cytokine is IL-1 or TNFα, or a fragment thereof.

78. The composition of claim 74, wherein said pro-apoptotic gene is a Fas pathway molecule, or a fragment thereof.

79. The composition of claim 78, wherein the Fas pathway molecule is Fas or FasL, or a fragment thereof.

80. A vector comprising a DNA template which encodes the siRNA of claim 72 or 73.

81. The vector of claim 80, wherein said vector is a lentiviral vector.

82. The vector of claim 80, wherein said vector is a retroviral vector.

83. A cell transfected with the vector of claim 80.

84. A method of inhibiting apoptosis in a cell comprising administering to the cell a composition of claim 72 which modulates apoptosis-related gene expression, thereby inhibiting apoptosis in a cell.

85. The method of claim 84, wherein said apoptosis-related gene is a Fas pathway molecule, or a fragment thereof.

86. The method of claim 85, wherein said Fas pathway molecule is Fas or FasL, or a fragment thereof.

87. The method of claim 84, wherein said cell is a hepatocyte, a T-cell, a hematopoietic cell, a neural cell, or a malignant cell.

88. A method of treating or preventing an apoptosis-mediated disease or disorder in a subject comprising administering to said subject a therapeutically or prophylactically effective amount of an siRNA which modulates apoptosis-related gene expression so that expression of said apoptosis-related gene is inhibited.

89. The method of claim 88, wherein said apoptosis-related gene expression is inhibited.

90. The method of claim 88, wherein said apoptosis-related gene is a Fas pathway molecule, or a fragment thereof.

91. The method of claim 90, wherein said Fas pathway molecule is Fas or FasL, or a fragment thereof.

92. The method of claim 88, wherein the disease or disorder is an immune or inflammatory disease.

93. The method of claim 92, wherein said immune or inflammatory disease is hepatitis.

94. The method of claim 88, wherein said disease or condition is cancer.

95. The method of claim 94, wherein said cancer is a cancer of the liver.

96. The method of claim 88, wherein said disease or condition is cirrhosis.

97. The method of claim 88, wherein the disease or condition is transplant rejection.

98. The method of claim 88, wherein said subject is a human.

99. The method of claim 88, wherein the disease or disorder is allograft rejection.
